(19) United States
(12) Patent Application Publication
(10) Pub. No.: US 2026/0062440 A1
(43) Pub. Date: Mar. 5, 2026

(54) Multiplex Dye Compounds

(71) Applicant: Life Technologies Corporation, Carlsbad, CA (US)

(72) Inventors: Mohammad Mehdi Zahedi, Dublin, CA (US); Brian Evans, Mountain View, CA (US); Khairuzzaman Bashar Mullah, Union City, CA (US); Carmen Gjerstad, Millbrae, CA (US); Mark Shannon, San Francisco, CA (US); Harrison Leong, San Francisco, CA (US)

(21) Appl. No.: 18/877,895

(22) PCT Filed: Jun. 29, 2023
(86) PCT No.: PCT/US2023/026642

§ 371 Date: Feb. 24, 2025

Related U.S. Application Data
(60) Provisional application No. 63/356,863, filed on Jun. 29, 2022.
(60) Provisional application No. 63/356,874, filed on Jun. 29, 2022.
(60) Provisional application No. 63/408,665, filed on Sep. 21, 2022.
(60) Provisional application No. 63/453,546, filed on Mar. 21, 2023.

(51) Int. Cl. C07H 21/04

(52) U.S. Cl. CPC C07H 21/04

(57) ABSTRACT

Described herein, inter alia, are multiplex dye compounds and methods of use thereof.

### CROSS REFERENCE TO RELATED APPLICATIONS

This application claims priority to International Application No. PCT/US2023/026642, filed Jun. 29, 2023 which claims priority to U.S. Provisional Application No. 63/356,863, filed Jun. 29, 2022; U.S. Provisional Application No. 63/356,874, filed Jun. 29, 2022; U.S. Provisional Application No. 63/408,665, filed Sep. 21, 2022; and U.S. Provisional Application No. 63/453,546, filed Mar. 21, 2023; each of which is hereby incorporated by reference in its entirety and for all purposes.

### BACKGROUND

Current qPCR probe design has the reporter dye on the 5′ end and a quencher molecule at the 3′ end. There are typically 20 to 30 bases between the fluorophore and the quencher. An internal quencher can be placed closer to the fluorophore dye within 6-12 bases between the reporter dye and quencher. This shortened length can provide better quenching and lower background which, in turn, improves accuracy and robustness of the assay. Disclosed herein, inter alia, are solutions to these and other problems in the art.

### BRIEF SUMMARY

In an aspect is provided a compound, or a salt thereof, having the formula:

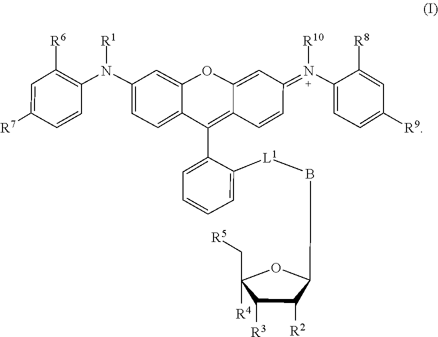

B is a divalent nucleobase.

L¹ is a divalent linker.

R² is hydrogen or —OR^{2A}.

R³ is —OR^{3A} or —O—P(NR^{3B}R^{3C})—OR^{3A}.

R⁴ is hydrogen or unsubstituted methyl, or R² and R⁴ substituents are joined to form a substituted or unsubstituted heterocycloalkyl.

R⁵ is —OR^{5A}.

R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl.

R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹ and R⁶ may be joined to form a substituted or unsubstituted heterocycloalkyl or substituted or unsubstituted heteroaryl.

R⁸ and R¹⁰ may be joined to form a substituted or unsubstituted heterocycloalkyl or substituted or unsubstituted heteroaryl.

R^{2A}, R^{3A}, R^{3B}, R^{3C}, R^{5A}, and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided an oligonucleotide, or a salt thereof, having the formula:

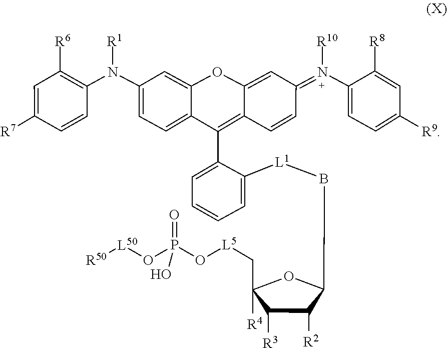

B, L¹, R¹, R², R⁴, R⁶, R⁷, R⁸, R⁹, and R¹⁰ are as described herein, including in embodiments.

L⁵ is a divalent oligonucleotide linker including from 4 to 40 nucleotides.

L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R⁵⁰ is a detectable moiety.

R³⁰ is —OR^{30A} or —O—P(NR^{3B}R^{3C})—OR^{3A}.

R^{30A} is hydrogen, a 3′ blocking moiety, or a monovalent oligonucleotide moiety.

R^{3A}, R^{3B}, and R^{3C} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a method of forming a double-stranded nucleic acid, the method including contacting a target oligonucleotide with a nucleic acid probe including a quencher moiety (e.g., internal quencher moiety), thereby forming the double-stranded nucleic acid; wherein the nucleic acid probe has the formula:

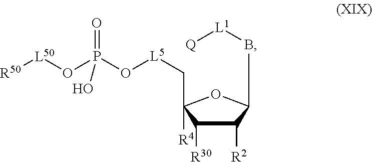

or a salt thereof. B, L¹, L⁵, L⁵⁰, R², R⁴, and R⁵⁰ are as described herein, including in embodiments. R³⁰ is —OR^{30A}, wherein R^{30A} is a monovalent oligonucleotide moiety. Q is a quencher moiety (e.g., internal quencher moiety).

In embodiments, Q is a quencher moiety having the formula:

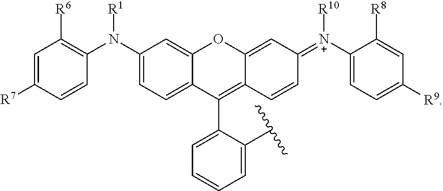

R¹, R⁶, R⁷, R⁸, R⁹, and R¹⁰ are as described herein, including in embodiments.

In an aspect is provided a method of detecting nucleic acids in a sample, including: (i) providing a reaction mixture, the reaction mixture including: at least a portion of the sample, a first probe detectably labeled with a first label configured to generate a first fluorescence signal, a second probe detectably labeled with a second label configured to generate a second fluorescence signal, wherein the first and second probes have different sequences, wherein the first and second labels are identical and/or generate substantially identical fluorescence; and wherein the second probe has the formula:

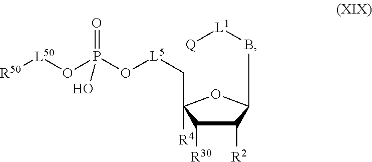

or a salt thereof, wherein Q, B, L¹, L⁵, L⁵⁰, R², and R⁴ are as described herein, including in embodiments; R³⁰ is —OR^{30A}, wherein R^{30A} is a monovalent oligonucleotide moiety; R⁵⁰ is the second label; (ii) subjecting the reaction mixture to an amplification process comprising a first set of reaction conditions and a second set of reaction conditions, the first set of reaction conditions being different than the second set of reaction conditions; and (iii) determining the presence of the first and/or second nucleic acid targets in the sample by measuring a fluorescence signal during the first set of reaction conditions, the fluorescence signal during the first set of reaction conditions being correlated with specific interaction or lack of interaction between the first probe and a first nucleic acid target, measuring a fluorescence signal during the second set of reaction conditions, the fluorescence signal during the second set of reaction conditions being correlated with specific interaction or lack of interaction between the first probe and a first nucleic acid target and with specific or lack of interaction between the second probe and a second nucleic acid target, and estimating the presence and/or amount of the first nucleic acid target and second nucleic acid target.

In an aspect is provided a method of detecting nucleic acids in a sample, including: (i) providing a reaction mixture, the reaction mixture including: at least a portion of the sample, a first probe detectably labeled with a first label configured to generate a first fluorescence signal that is indicative of the presence or absence of a first nucleic acid target, a second probe detectably labeled with a second label configured to generate a second fluorescence signal that is indicative of the presence or absence of a second nucleic acid target, wherein the first and second probes have different sequences, wherein the first and second labels are identical and/or generate substantially identical fluorescence; and wherein the second probe has the formula:

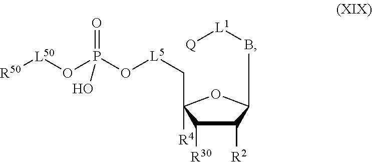

or a salt thereof, wherein Q, B, L¹, L⁵, L⁵⁰, R², and R⁴ are as described herein, including in embodiments; R³⁰ is —OR^{30A}, wherein R^{30A} is a monovalent oligonucleotide moiety; R⁵⁰ is the second label; (ii) subjecting the reaction mixture to an amplification process including a first set of reaction conditions and a second set of reaction conditions, the first set of reaction conditions being different than the second set of reaction conditions; and (iii) determining any presence and/or amount of the first and/or second nucleic acid targets in the reaction mixture by measuring during the first set of reaction conditions a first total fluorescence signal that includes any first fluorescence signal if present and any second fluorescence signal if present, measuring during the second set of reaction conditions a second total fluorescence signal including any first fluorescence signal if present and any second fluorescence signal if present, and estimating the first fluorescence signal and/or second fluorescence signal based on the first and second total fluorescence signals.

In an aspect is provided a method of detecting nucleic acids in a sample, including: providing a reaction mixture, the reaction mixture including a primer pair complementary to a nucleic acid target or its complement for generating an amplicon, and a non-cleavable probe configured to hybridize to the amplicon, the probe including a detectable label configured to provide a fluorescent signal that corresponds to an amount of generated amplicon, subjecting the reaction mixture to an amplification process to generate the amplicons, wherein the label generates fluorescence without cleavage of the probe during the amplification process, and wherein the amplification process utilizes a series of thermal cycling steps that includes at least three different target temperatures; and measuring the fluorescent signal from the probe; wherein the probe has the formula:

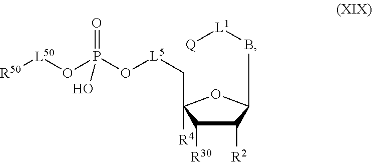

or a salt thereof, wherein Q, B, L¹, L⁵, L⁵⁰, R², and R⁴ are as described herein, including in embodiments; R³⁰ is —OR^{30A}, wherein R^{30A} is a monovalent oligonucleotide moiety; and R⁵⁰ is the detectable label.

In an aspect is provided a method of detecting nucleic acids in a sample, including: providing a reaction mixture, the reaction mixture including a primer pair targeted to a nucleic acid target for generating an amplicon, the primer pair including a tailed primer and a non-tailed primer provided at different concentrations, and a non-cleavable probe configured to hybridize to the amplicon, the probe including a detectable label configured to generate a fluorescent signal that corresponds to an amount of generated amplicon, subjecting the reaction mixture to an amplification process to generate the amplicons, wherein the probe generates fluorescence without being cleaved during the amplification process; and measuring the fluorescent signal from the probe; wherein the probe has the formula:

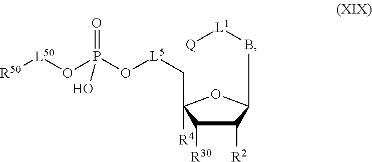

or a salt thereof, wherein Q, B, L¹, L⁵, L⁵⁰, R², and R⁴ are as described herein, including in embodiments; R³⁰ is —OR^{30A}, wherein R^{30A} is a monovalent oligonucleotide moiety; and R⁵⁰ is the detectable label.

In an aspect is provided a method of detecting the presence or amount of a first and/or second target in a reaction mixture, including: including a first probe and a second probe in the reaction mixture, wherein the first probe can specifically interact with a first target and includes a first label that can produce a first detectable signal, and the second probe can specifically interact with a second target and includes a second label that can produce a second detectable signal; wherein the second probe has the formula:

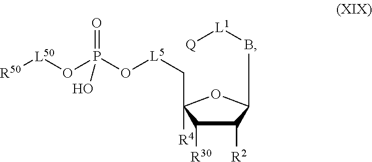

or a salt thereof, wherein Q, B, L¹, L⁵, L⁵⁰, R², and R⁴ are as described herein, including in embodiments; R³⁰ is —OR^{30A}, wherein R^{30A} is a monovalent oligonucleotide moiety; and R⁵⁰ is the second label; allowing specific interaction of the first and second probe with any first and second target, respectively, in the reaction mixture; measuring a first total signal through an optical filter under a first set of conditions, wherein the first total signal includes the first and second detectable signals from the first and second labels, and wherein under the first set of conditions, the first detectable signal is increased as a result of specific interaction of the first probe with the first target, but the second detectable signal is not increased as a result of specific interaction of the second probe with the second target; measuring a second total signal through the same optical filter under a second set of conditions, wherein the second total signal includes the first and second detectable signals from the first and second labels, and wherein under the second set of conditions, the second detectable signal is increased as a result of specific interaction of the second probe with the second target; and assessing the presence or amount of the first and/or second target, by estimating the first detectable signal and the second detectable signal based on both the first total signal and the second total signal.

### BRIEF DESCRIPTION OF THE DRAWINGS

FIGS. 1A-1B. FIG. 1A illustrates emission spectra for various fluorescent dyes commonly used in nucleic acid detection assays and their associated detection channels. FIG. 1B illustrates emission spectra for various fluorescent dye commonly used in nucleic acid detection assays and their associated detection channels, showing that “Dye 1” and “Dye 2” may be detected within the same channel.

FIGS. 2A-2B. FIG. 2A is a schematic overview of a method for detecting multiple target nucleic acids within the same detection channel by providing different first and second probe types, varying the reaction mixture conditions, and measuring the resulting total signal at each set of conditions. FIG. 2B is a graph showing signal response over time for the method outlined in FIG. 2A when the reaction mixture is cycled between a first set of reaction conditions and a second set of reaction conditions.

FIGS. 3A-3B. FIG. 3A illustrates fluorescence activity of a cleavable probe (e.g., TaqMan probe) and a non-cleavable probe (e.g., an extendable fluorogenic (EF) probe) during annealing, extension, and denaturation steps of a thermal cycle, showing that the cleavable probe increases the fluorescence signal by releasing and unquenching the corresponding label, whereas the non-cleavable probe increases the fluorescence signal when a duplex amplicon is formed during extension. FIG. 3B is a graph showing fluorescent signal response over time during thermal cycling of an amplification process that utilizes the cleavable and non-cleavable probes of FIG. 3A to implement intra-channel multiplexing.

FIGS. 4A-4C. FIG. 4A illustrates a process of using a tailed primer, specific to a nucleic acid target, to form a template to which the EF probe can hybridize. FIG. 4B illustrates an example tailed forward primer, reverse primer, and EF probe that may be included in the reaction mixture to implement the process of FIG. 4A. FIG. 4C illustrates a three-stage thermal cycling method that may be utilized during an amplification process involving EF probes and optionally TaqMan probes.

FIGS. 5A-5B provide an overview of a method for detecting multiple target nucleic acids within the same detection channel in a digital polymerase chain reaction (dPCR) application.

FIGS. 6A-6F. FIG. 6A illustrates fluorescent signals of a TaqMan probe and an extendable fluorogenic (EF) probe at extension and denaturation steps. FIGS. 6B-6D illustrate results of a duplex assay test in which TaqMan and EF probes were designed to generate fluorescence signals in the same dye channel (FIG. 6B) or in different dye channels (FIGS. 6C-6D). FIG. 6E compares the EF-associated fluorescence signal after baseline adjustment (dRn) as derived using the results of the assay of FIG. 6B with the EF-associated fluorescence signal as directly measured in the assay of FIG. 6C, the results showing close correlation and therefore demonstrating that fluorescent signals attributable to different probe types within the same detection channel can be separately resolved. FIG. 6F compares the TaqMan-associated fluorescence signal after baseline adjustment (dRn).

FIG. 7 illustrates the results of another assay test that included 5 different detection channels/dyes, each with a corresponding TaqMan probe and an EF probe, the results showing that the fluorescent signals of the different probe types can be separately resolved, and showing that a 9-plex reaction can be effectively carried out.

FIG. 8 is a plot comparing the endpoint signal of partitions at 65° C. and at 95° C. following a dPCR process, showing that the partition signals fall into identifiable clusters that allow for estimation of concentration of different targets.

FIG. 9. Chemical structures of QSY7-dU-Amidite (top) and QSY21-dU-Amidite (bottom).

FIG. 10. Sample list of fluorophores and compatible quenchers (shaded box=compatible).

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

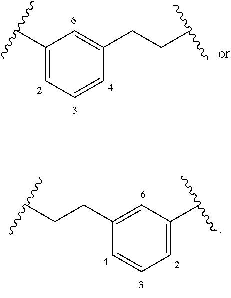 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″ R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″ R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

In the context of a nucleic acid probe and a target nucleic acid, the term “specifically interact” (and similar terms) indicates that the probe is designed to interact with the target to a greater degree than with non-target nucleic acids also present in the reaction mixture. For example, specific interaction may include hybridization of the probe, in whole or in part, with the corresponding target. The hybridization between the probe and target need not be 100%. For example, functionally effective interaction may be accomplished with probes having homology to their respective target of at least about 70%, at least about 75%, at least about 80%, at least about 85%, at least about 90%, at least about 95%, at least about 97%, at least about 98%, at least about 99%, or up to 100%.

As used herein, a “detection channel” is a specified subset of the total range of possible values of detectable signals. For example, where the detectable signals are fluorescence signals, a detection channel (i.e., fluorescence channel or dye channel) can represent a wavelength band of specified size. A detection channel may, for example, have a band size of about 10-60 nm, depending on instrument features such as sensitivity and/or desired signal granularity. A detection channel can further include a discontinuous wavelengths or wavelength ranges. A detection channel may additionally or alternatively be defined according to the optical filter arrangement used to measure the detectable signals. Each different detection channel typically comprises a specific optical filter arrangement to block non-channel emissions. Thus, as a functional definition, each detectable signal within a given optical filter arrangement may be considered as being within the same detection channel. In some instances, different fluorescent labels (e.g., different chemical structures) are nonetheless detected with the same detection channel. As an example, the fluorescent dyes Cy5 and Alexa647 provide similar emission wavelengths and may be detected within the same channel.

As used herein, “substantially identical” signals are signals that are not clearly distinguishable from each other under the detection conditions being used. Optionally, the emission spectra of two substantially identical signals overlap to such an extent that each signal cannot be separately detected, such as where the composite emission spectrum does not show the presence of two distinct peaks. Optionally, “substantially identical fluorescence” emissions can be within similar wavelength bands. For example, a first fluorescence signal and a second fluorescence signal with substantially identical fluorescence may have emission peaks that differ by no more than about 10 nm, or no more than about 8 nm, or no more than about 6 nm, or no more than about 4 nm, or no more than about 2 nm, or no more than about 1 nm, or are substantially indistinguishable from one another by the detection instrument used to measure the fluorescence emissions. Additionally, or alternatively, fluorescence signals may be considered to have “substantially identical fluorescence” in applications where they are measured using the same detection apparatus, such as the same optical filter arrangement. In an embodiment, the substantially identical signals have substantially identical excitation/absorbance spectra, such that they cannot be subjected to excitation separately. Optionally, both labels are subjected to excitation during detection. Both labels can be simultaneously excited and/or detected.

As used herein with respect to signals, “substantial” indicates significantly above a background. For example, a “substantial signal” and/or a detectable signal that has “substantial fluorescence” is a signal significantly above a background (i.e., baseline) level, including a fluorescence signal that is significantly above a background/baseline level of fluorescence. This may be defined by a threshold value that separates background fluorescence from substantial fluorescence. The threshold value may vary according to particular testing protocols and application needs. In some embodiments (e.g., without a passive reference), the threshold is set at a ΔRn of about 1,000 to about 30,000, or about 2,000 to about 20,000, or about 3,000 to about 15,000 or about 4,000 to about 6,000, for example, or within a range having endpoints defined by any two of the foregoing values. In some embodiments (e.g., with a passive reference), the threshold is set at a ΔRn of about 0.01 to 0.5, for example. In some embodiments, the threshold value is some percentage above the baseline level, such as about 5 percent to about 10 percent above the baseline level.

A “background” or “baseline” level of signal (i.e., background/baseline level of fluorescence) during an amplification process may be determined according to methods known to those of skill in the art. As a non-limiting example, the baseline level may be determined as the median signal of the amplification cycles before exponential amplification occurs. For example, exponential amplification may be determined when the change in signal from one amplification cycle to the next exceeds a certain percentage indicative of exponential change.

As a corollary, a signal and/or fluorescence level that is not “substantial” according to the foregoing may be described herein as “negligible.” Similarly, with respect to probe binding, a probe is “substantially bound” to its target when it is bound significantly above background (e.g., above binding to a non-target). Optionally, at least 1%, 5%, 10%, 20%, 50% or 80% of the probe or the target is bound.

As used herein, a “cleavable” probe is a probe that is intended to be cleaved as a result of specific interaction of the probe with its respective target, and to cause a release of the corresponding label and an increase in the corresponding detectable signal as a result.

As used herein, a “non-cleavable” probe is a probe with a label that is intended to remain associated with the probe throughout the assay. In a non-cleavable probe, the corresponding detectable signal varies according to configuration changes of the probe rather than by release of the label from the probe. An extendable fluorogenic probe, such as a universal or hairpin extendable fluorogenic probe, as described in various embodiments, is an example of a non-cleavable probe.

The terms “detectable signal” and “label signal” are used synonymously herein. For example, a “first label signal” is the signal emitted by a first label of a first probe type and a “second label signal” is the signal emitted by a second label of a second probe type. A “total signal” is the total measured signal within a particular detection channel at a given time point or measurement point. Multiple different “detectable signals”/“label signals” may contribute to the same “total signal.” For example, a total signal may include signal generated by a first label of a first probe type and signal generated by a second label of a second probe type. In some embodiments, the signals are fluorescence signals, and terms such as “first fluorescence signal,” “second fluorescence signal,” and “total fluorescence signal” may be used as specific examples of the corresponding broader terms.

The terms “determine,” “calculate,” and “estimate” are used synonymously herein.

These terms are not intended to imply an exact level of measurement precision. Thus, where a value is “determined,” “calculated,” or “estimated” using the embodiments described herein, it will be understood that such a value may include some degree of inherent error due to factors such as detection instrument tolerances, rounding, chemical reaction variability, and other inherent measurement imperfections known and understood by those of skill in the art.

For any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques. In embodiments, about means within a standard deviation using measurements generally acceptable in the art. In embodiments, about means a range extending to +/−10% of the specified value. In embodiments, about includes the specified value.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, 99Mo, ¹⁰⁵Pd, ¹⁰⁵Rh ¹¹¹Ag, ¹¹¹n, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. In embodiments, a detectable moiety is a moiety (e.g., monovalent form) of a detectable agent. In embodiments, a detectable label moiety is a moiety (e.g., monovalent form) of a detectable label.

The terms “fluorophore” or “fluorescent agent” or “fluorescent dye” are used interchangeably and refer to a substance, compound, agent (e.g., a detectable agent), or composition (e.g., compound) that can absorb light at one or more wavelengths and re-emit light at one or more longer wavelengths, relative to the one or more wavelengths of absorbed light. Examples of fluorophores that may be included in the compounds and compositions described herein include fluorescent proteins, xanthene derivatives (e.g., fluorescein, rhodamine, Oregon green, eosin, or Texas red), cyanine and derivatives (e.g., cyanine, indocarbocyanine, oxacarbocyanine, thiacarbocyanine, or merocyanine), napththalene derivatives (e.g., dansyl or prodan derivatives), coumarin and derivatives, oxadiazole derivatives (e.g., pyridyloxazole, nitrobenzoxadiazole or benzoxadiazole), anthracene derivatives (e.g., anthraquinones, DRAQ5, DRAQ7, or CyTRAK Orange), pyrene derivatives (e.g., cascade blue and derivatives), oxazine derivatives (e.g., Nile red, Nile blue, cresyl violet, or oxazine 170), acridine derivatives (e.g., proflavin, acridine orange, acridine yellow), arylmethine derivatives (e.g., auramine, crystal violet, or malachite green), tetrapyrrole derivatives (e.g., porphin, phthalocyanine, bilirubin), CF Dye™, DRAQ™, CyTRAK™ BODIPY™, Alexa Fluor™, DyLight Fluor™, Atto™, Tracy™, FluoProbes™, Abberior Dyes™, DY™ dyes, MegaStokes Dyes™, Sulfo Cy™, Seta™ dyes, SeTau™ dyes, Square Dyes™, Quasar™ dyes, Cal Fluor™ dyes, SureLight Dyes™, PerCP™, Phycobilisomes™ APC™, APCXL™, RPE™, and/or BPE™. A fluorescent moiety is a radical of a fluorescent agent. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners). In embodiments, the fluorophore is an aromatic (e.g., polyaromatic) moiety having a conjugated π-electron system. In embodiments, the fluorophore is a fluorescent dye moiety, that is, a monovalent fluorophore.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Examples of detectable agents include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent moiety or fluorescent dye moiety. In embodiments, the detectable label is a fluorescent dye. In embodiments, the detectable label is a fluorescent dye capable of exchanging energy with another fluorescent dye (e.g., fluorescence resonance energy transfer (FRET) chromophores).

As used herein, the term “quencher” refers to a substance, compound, agent, or composition (e.g., compound) that is capable of absorbing energy from a detectable agent (e.g., a fluorophore or a fluorescent dye) and re-emitting at least a portion of the energy, e.g., as either heat (e.g., in the case of dark quenchers) or visible light (e.g., in the case of fluorescent quenchers). In embodiments, the quencher absorbs energy from a fluorophore and re-emits at least a portion of the energy as heat in the case of dark quenchers. In embodiments, the quencher absorbs energy from a fluorophore and re-emits at least a portion of the energy as visible light in the case of fluorescent quenchers. A “quencher moiety” is a monovalent form of a quencher. An “internal quencher moiety” refers to a quencher moiety that is placed at an internal site of a probe.

As used herein, the term “salt” refers to acid or base salts of the compounds described herein. Thus, the compounds of the present invention may exist as salts. The present invention includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium salts. In embodiments, compounds may be presented with a positive charge, and it is understood an appropriate counterion (e.g., chloride ion, fluoride ion, or acetate ion) may also be present, though not explicitly shown.

Likewise, for compounds having a negative charge (e.g.,

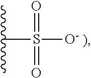 [SMILES: *S(=O)(=O)[O-]]

it is understood an appropriate counter-ion (e.g., a proton, sodium ion, potassium ion, or ammonium ion) may also be present, though not explicitly shown. The protonation state of the compound (e.g., a compound described herein) depends on the local environment (i.e., the pH of the environment). Therefore, in embodiments, the compound may be described as having a moiety in a protonated state (e.g.,

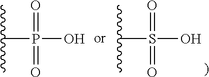

or an ionic state (e.g.,

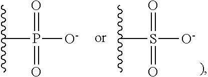

and it is understood these are interchangeable. In embodiments, the counterion is represented by the symbol A (e.g., A⁺ or A⁻).

The neutral forms of the compounds are preferably regenerated by contacting the salt with a base or acid and isolating the parent compound in the conventional manner. The parent form of the compound may differ from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated, however, that the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a protein or enzyme. In embodiments, contacting includes allowing a compound described herein to interact with a protein or enzyme that is involved in a signaling pathway.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. In certain embodiments the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see, Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. A residue of a nucleic acid, as referred to herein, is a monomer of the nucleic acid (e.g., a nucleotide). The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. A “nucleic acid moiety” as used herein is a monovalent form of a nucleic acid. In embodiments, the nucleic acid moiety is attached to the 3′ or 5′ position of a nucleotide or nucleoside.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

“Nucleotide,” as used herein, refers to a nucleoside-5′-phosphate (e.g., polyphosphate) compound, or a structural analog thereof, which can be incorporated (e.g., partially incorporated as a nucleoside-5′-monophosphate or derivative thereof) by a nucleic acid polymerase to extend a growing nucleic acid chain (such as a primer). Nucleotides may comprise bases such as adenine (A), cytosine (C), guanine (G), thymine (T), uracil (U), or analogues thereof, and may comprise 1, 2, 3, 4, 5, 6, 7, 8, or more phosphates in the phosphate group. Nucleotides may be modified at one or more of the base, sugar, or phosphate group. A nucleotide may have a label or tag attached (a “labeled nucleotide” or “tagged nucleotide”). In embodiments, the nucleotide is a deoxyribonucleotide. In embodiments, the nucleotide is a ribonucleotide. In embodiments, nucleotides comprise 3 phosphate groups (e.g., a triphosphate group).

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

A “nucleoside” is structurally similar to a nucleotide, but is missing the phosphate moieties that are present in a nucleotide. An example of a nucleoside analogue would be one in which the label is linked to the base and there is no phosphate group attached to the sugar molecule. “Nucleoside,” as used herein, refers to a glycosyl compound consisting of a nucleobase and a 5-membered ring sugar (e.g., either ribose or deoxyribose). Nucleosides may comprise bases such as adenine (A), cytosine (C), guanine (G), thymine (T), uracil (U), or analogues thereof. Nucleosides may be modified at the base and/or and the sugar. In embodiments, the nucleoside is a deoxyribonucleoside. In embodiments, the nucleoside is a ribonucleoside.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity.

The term “polymerase,” as used herein, refers to any natural or non-naturally occurring enzyme or other catalyst that is capable of catalyzing a polymerization reaction, such as the polymerization of nucleotide monomers to form a nucleic acid polymer. Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9°N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9°N polymerase, 9°N polymerase (exo-) A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator 7, 9°N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). As used herein, the term “thermophilic nucleic acid polymerase” refers to a family of DNA polymerases (e.g., 9°N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L408S/Y409A/P410V mutations, NEB Therminator IX DNA polymerase), or 7-phosphate labeled nucleotides (e.g., Therminator γ: D141A/E143A/W355A/L408W/R460A/Q461S/K464E/D480V/R484W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996).

The term “end-point” as referring to a cycle is a designated cycle at which the PCR process is assumed to be completed and/or a designated cycle at which a signal threshold that is above background signal by a defined amount occurs. In various embodiments, an end-point cycle in accordance with the present disclosure may range from 20 to 45 cycles, for example, from 30-40 cycles. However, the number of cycles to an end-point cycle may change. For example, the number of cycles to an end-point cycle may be correlated to where the emission (e.g., fluorescence) signal indicative of amplification product reaches an approximate plateau. An “end-point signal” refers to an emission signal measured during an end-point cycle.

The term “spectral similarity” refers to the emission signal of detectable labels that have the same spectral profile or a substantially overlapping spectral profile. Thus, different probe types carrying the same detectable label or different probe types carrying different detectable labels with substantial spectral overlap in emission signal can both be considered probes with spectral similarity. In some implementations, detectable labels having spectral similarity can be detectable in a same optical detection channel, but other techniques can be used as well to detect the emission signals of such detectable labels. References made to substantially overlapping spectra should be understood to mean spectral similarity.

### II. Compounds

In an aspect is provided a compound, or a salt thereof, having the formula:

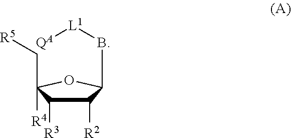

Q^{A} is a quencher moiety.

B is a divalent nucleobase.

L¹ is a divalent linker.

R² is hydrogen or —OR^{2A}.

R³ is —OR^{3A} or —O—P(NR^{3B}R^{3C})—OR^{3A}.

R⁴ is hydrogen or unsubstituted methyl, or R² and R⁴ substituents are joined to form a substituted or unsubstituted heterocycloalkyl (e.g., 3 to 8 membered, 3 to 6 membered, 4 to 6 membered, 4 to 5 membered, or 5 to 6 membered).

R⁵ is —OR^{5A}.

R^{2A}, R^{3A}, R^{3B}, R^{3C}, and R^{5A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, the quencher moiety is a monovalent form of QSY7. In embodiments, the quencher moiety is a monovalent form of

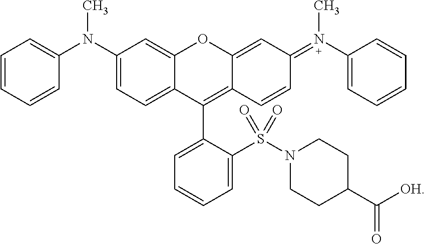

In embodiments, the quencher moiety is

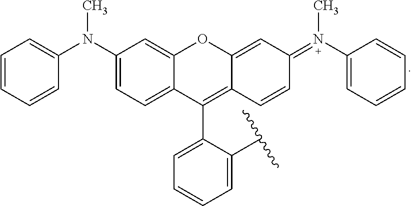

In embodiments, the quencher moiety is a monovalent form of QSY21. In embodiments, the quencher moiety is a monovalent form of

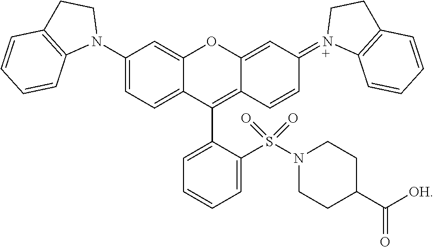

In embodiments, the quencher moiety is

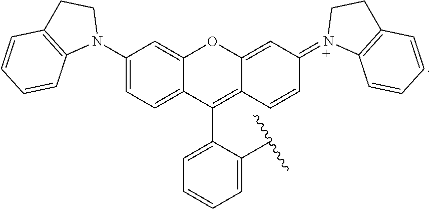

In embodiments, the quencher moiety is a monovalent form of QSY9. In embodiments, the quencher moiety is a monovalent form of

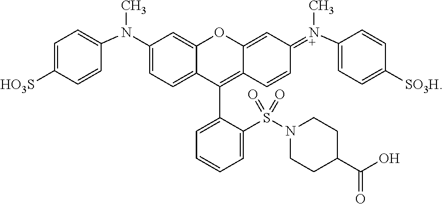

In embodiments, the quencher moiety is

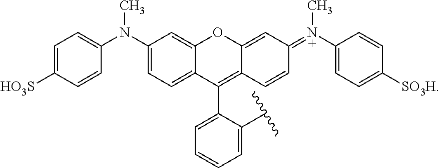

In embodiments, the quencher moiety is a monovalent form of BHQ1. In embodiments, the quencher moiety is a monovalent form of

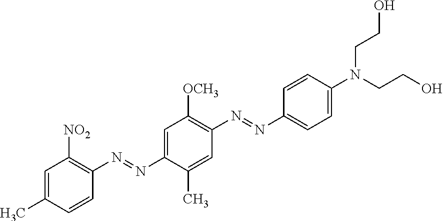 [SMILES: COc1cc(/N=N/c2ccc(C)cc2[N+](=O)[O-])c(C)cc1/N=N/c1ccc(N(CCO)CCO)cc1]

In embodiments, the quencher moiety is

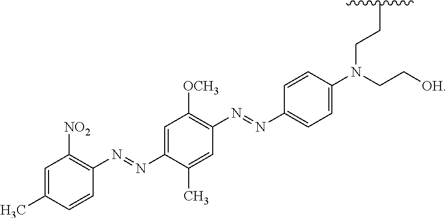 [SMILES: *CCN(CCO)c1ccc(/N=N/c2cc(C)c(/N=N/c3ccc(C)cc3[N+](=O)[O-])cc2OC)cc1]

In embodiments, the quencher moiety is a monovalent form of BHQ2. In embodiments, the quencher moiety is a monovalent form of

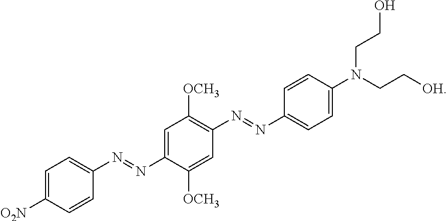 [SMILES: COc1cc(/N=N/c2ccc([N+](=O)[O-])cc2)c(OC)cc1/N=N/c1ccc(N(CCO)CCO)cc1]

In embodiments, the quencher moiety is

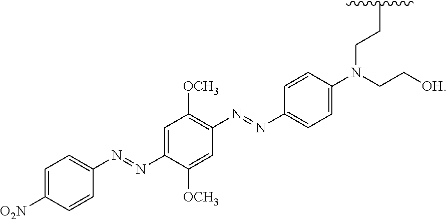 [SMILES: *CCN(CCO)c1ccc(/N=N/c2cc(OC)c(/N=N/c3ccc([N+](=O)[O-])cc3)cc2OC)cc1]

In embodiments, the quencher moiety is a monovalent form of BHQ3. In embodiments, the quencher moiety is a monovalent form of

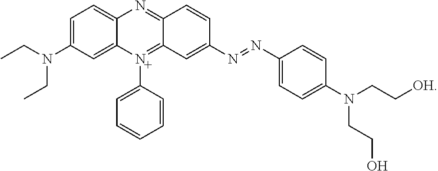

In embodiments, the quencher moiety is

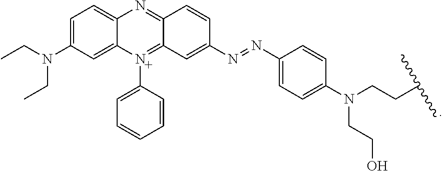

In embodiments, the quencher moiety is a monovalent form of Dabcyl. In embodiments, the quencher moiety is a monovalent form of

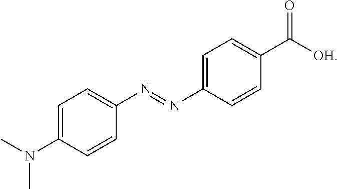 [SMILES: CN(C)c1ccc(/N=N/c2ccc(C(=O)O)cc2)cc1]

In embodiments, the quencher moiety is

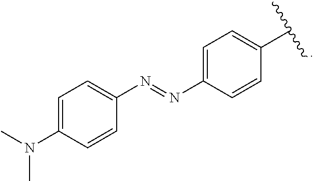 [SMILES: *c1ccc(/N=N/c2ccc(N(C)C)cc2)cc1]

In embodiments, the quencher moiety is a monovalent form of Dabsyl. In embodiments, the quencher moiety is a monovalent form of

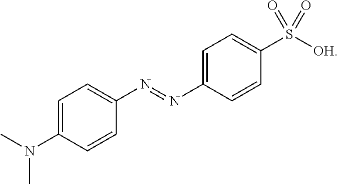 [SMILES: CN(C)c1ccc(/N=N/c2ccc(S(=O)(=O)O)cc2)cc1]

In embodiments, the quencher moiety is a monovalent form of Eclipse. In embodiments, the quencher moiety is a monovalent form of

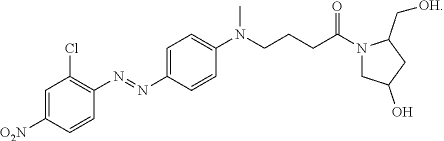 [SMILES: CN(CCCC(=O)N1CC(O)CC1CO)c1ccc(/N=N/c2ccc([N+](=O)[O-])cc2Cl)cc1]

In embodiments, the quencher moiety is

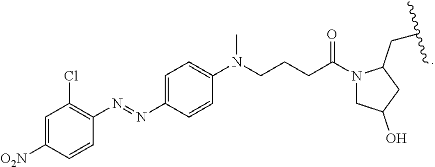 [SMILES: *CC1CC(O)CN1C(=O)CCCN(C)c1ccc(/N=N/c2ccc([N+](=O)[O-])cc2Cl)cc1]

In embodiments, the quencher moiety is a monovalent form of BBQ-650. In embodiments, the quencher moiety is

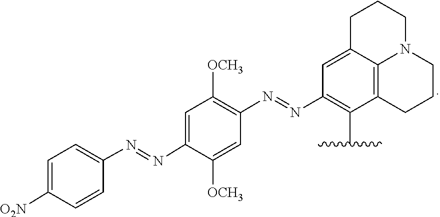 [SMILES: *c1c(/N=N/c2cc(OC)c(/N=N/c3ccc([N+](=O)[O-])cc3)cc2OC)cc2c3c1CCCN3CCC2]

In embodiments, the quencher moiety is a monovalent form of Iowa Black RQ. In embodiments, the quencher moiety is a monovalent form of Iowa Black FQ. In embodiments, the quencher moieties above are all interchangeable. In embodiments, the quencher moiety can be substituted in Formulae (I), (IA), (II), (III), (IV), (V), (VI), (VI-1), (VI-2), (VI-3), (VI-4), (VI-5), (VII), (VII-1), (VII-2), (VII-3), (VII-4), (VII-5), (VIII), (VIII-1), (VIII-2), (VIII-3), (VIII-4), (VIII-5), (IX), (IX-1), (IX-2), (IX-3), (IX-4), (IX-5), (X), (XI), (XII), (XIII), (XIV), (XV), (XV-1), (XV-2), (XV-3), (XV-4), (XV-5), (XVI), (XVI-1), (XVI-2), (XVI-3), (XVI-4), (XVI-5), (XVII), (XVII-1), (XVII-2), (XVII-3), (XVII-4), (XVII-5), (XVIII), (XVIII-1), (XVIII-2), (XVIII-3), (XVIII-4), (XVIII-5), and (XIX), and embodiments thereof.

In an aspect is provided a compound, or a salt thereof, having the formula:

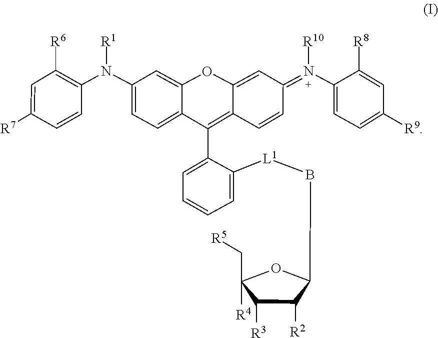

B is a divalent nucleobase.

L¹ is a divalent linker.

R² is hydrogen or —OR^{2A}.

R³ is —OR^{3A} or —O—P(NR^{3B}R^{3C})—OR^{3A}.

R⁴ is hydrogen or unsubstituted methyl, or R² and R⁴ substituents are joined to form a substituted or unsubstituted heterocycloalkyl (e.g., 3 to 8 membered, 3 to 6 membered, 4 to 6 membered, 4 to 5 membered, or 5 to 6 membered).

R⁵ is —OR^{5A}.

R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹ and R⁶ may be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

R⁸ and R¹⁰ may be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

R^{2A}, R^{3A}, R^{3B}, R^{3C}, R^{5A}, and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

A person having ordinary skill in the art would understand that the compound may exist as a neutral species with a counterion.

In embodiments, the compound has the formula:

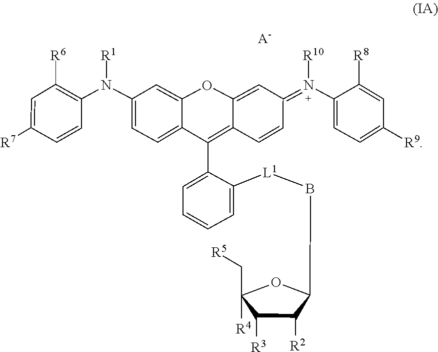

B, L¹, R², R³, and R⁵ are as described herein, including in embodiments. A⁻ is a counterion. In embodiments, A⁻ is Cl⁻. In embodiments, A⁻ is Br. In embodiments, A⁻ is H₃CC(O)O⁻. In embodiments, A⁻ is F₃CC(O)O⁻.

In embodiments, the compound has the formula.

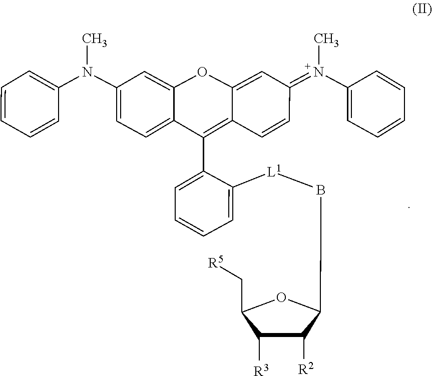

B, L¹, R², R³, and R⁵ are as described herein, including in embodiments.

In embodiments, the compound has the formula:

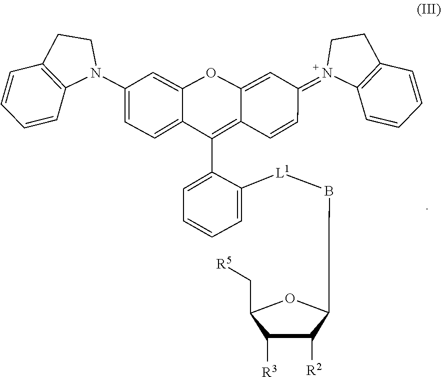

B, L¹, R², R³, and R⁵ are as described herein, including in embodiments.

In embodiments, the compound has the formula:

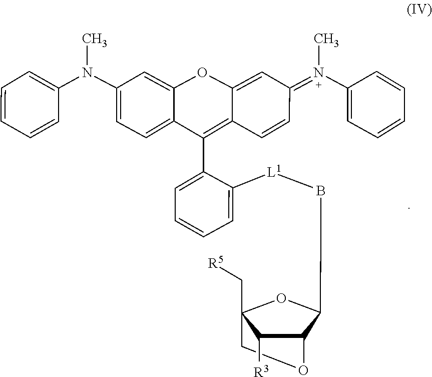

B, L¹, R³, and R⁵ are as described herein, including in embodiments.

In embodiments, the compound has the formula:

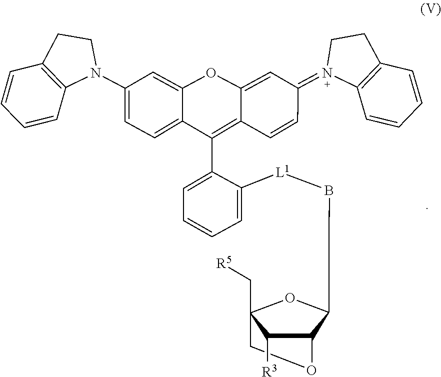

B, L¹, R³, and R⁵ are as described herein, including in embodiments.

In embodiments, B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof. In embodiments, B is a divalent cytosine or a derivative thereof, divalent guanine or a derivative thereof, divalent adenine or a derivative thereof, divalent thymine or a derivative thereof, or divalent uracil or a derivative thereof. In embodiments, B is a divalent cytosine or a derivative thereof. In embodiments, B is a divalent guanine or a derivative thereof. In embodiments, B is a divalent adenine or a derivative thereof. In embodiments, B is a divalent thymine or a derivative thereof. In embodiments, B is a divalent uracil or a derivative thereof.

In embodiments, the compound has the formula:

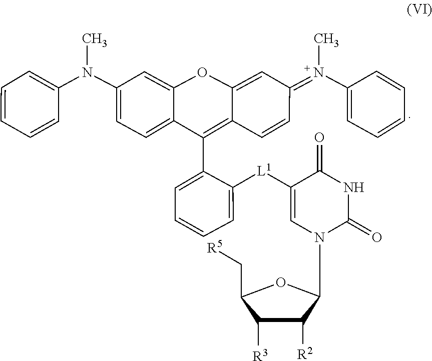

L¹, R², R³, and R⁵ are as described herein, including in embodiments.

In embodiments, the compound has the formula:

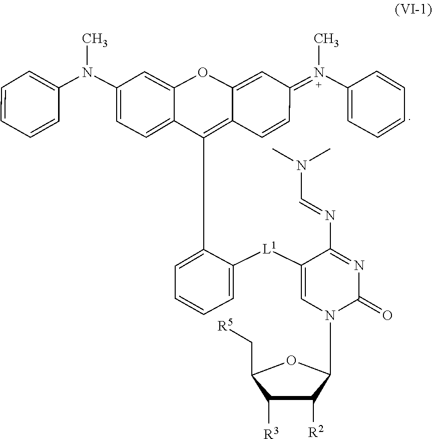

L¹, R², R³, and R⁵ are as described herein, including in embodiments.

In embodiments, the compound has the formula:

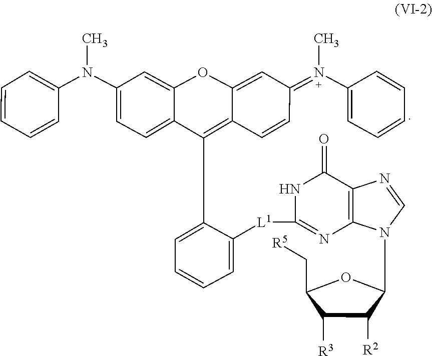

L¹, R², R³, and R⁵ are as described herein, including in embodiments.

In embodiments, the compound has the formula:

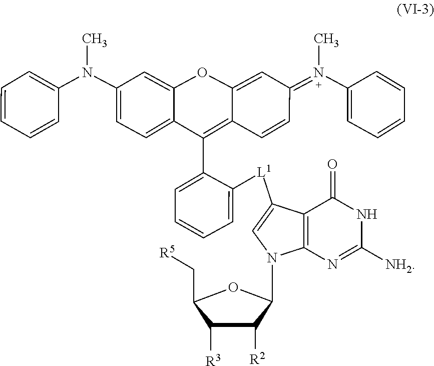

L¹, R², R³, and R⁵ are as described herein, including in embodiments.

In embodiments, the compound has the formula:

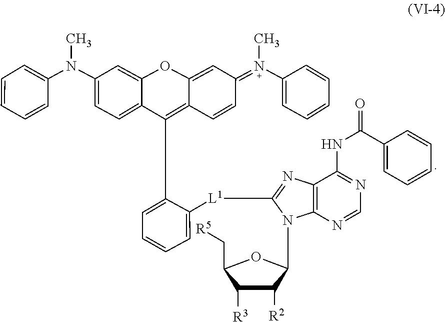

L¹, R², R³, and R⁵ are as described herein, including in embodiments.

In embodiments, the compound has the formula:

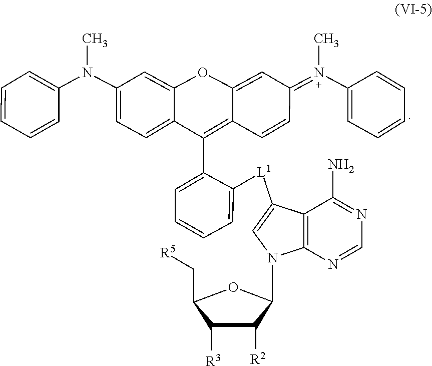

L¹, R², R³, and R⁵ are as described herein, including in embodiments.

In embodiments, the compound has the formula:

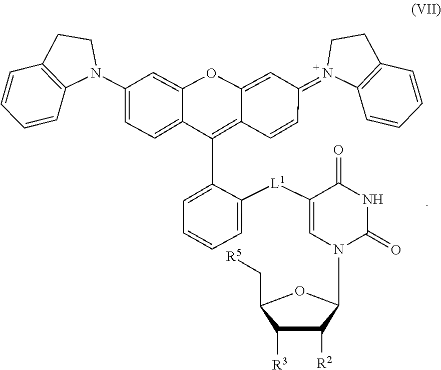

L¹, R², R³, and R⁵ are as described herein, including in embodiments.

In embodiments, the compound has the formula:

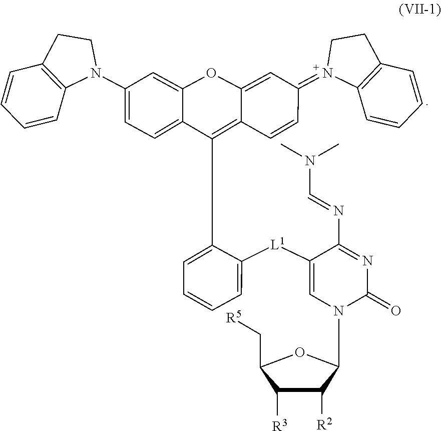

L¹, R², R³, and R⁵ are as described herein, including in embodiments.

In embodiments, the compound has the formula:

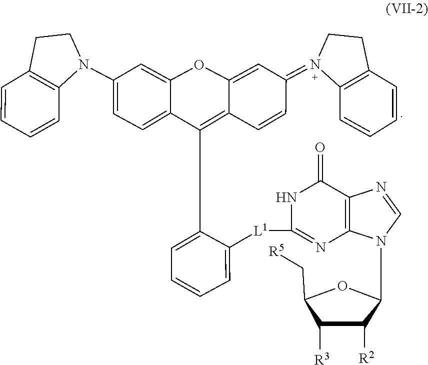

L¹, R², R³, and R⁵ are as described herein, including in embodiments.

In embodiments, the compound has the formula:

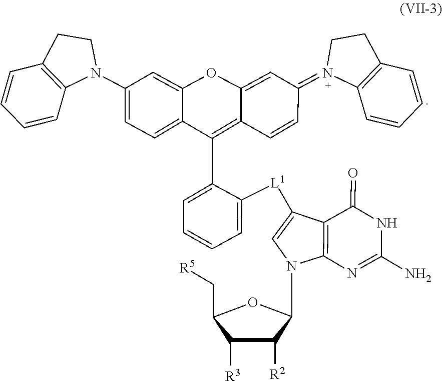

L¹, R², R³, and R⁵ are as described herein, including in embodiments.

In embodiments, the compound has the formula:

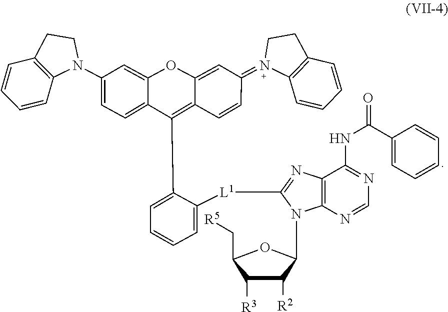

L¹, R², R³, and R⁵ are as described herein, including in embodiments.

In embodiments, the compound has the formula:

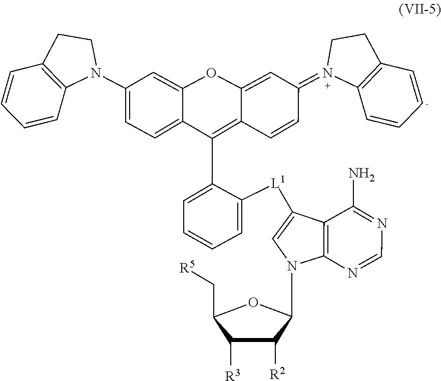

L¹, R², R³, and R⁵ are as described herein, including in embodiments.

In embodiments, the compound has the formula:

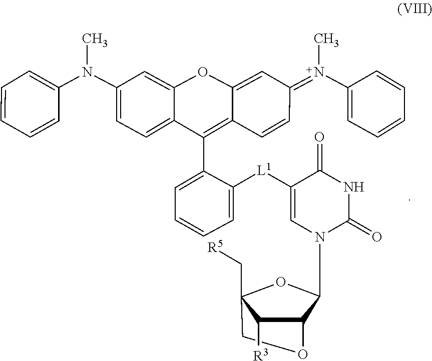

L¹, R³, and R⁵ are as described herein, including in embodiments.

In embodiments, the compound has the formula:

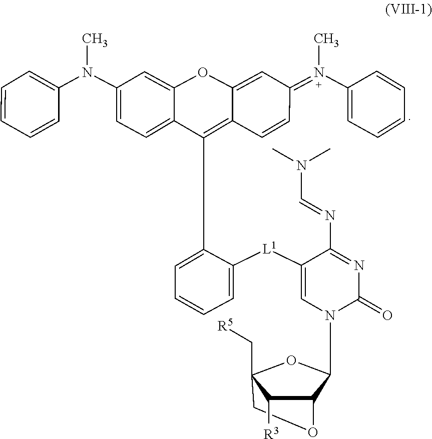

L¹, R³, and R⁵ are as described herein, including in embodiments.

In embodiments, the compound has the formula:

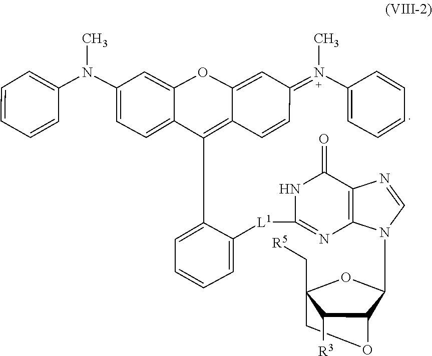

L¹, R³, and R⁵ are as described herein, including in embodiments.

In embodiments, the compound has the formula:

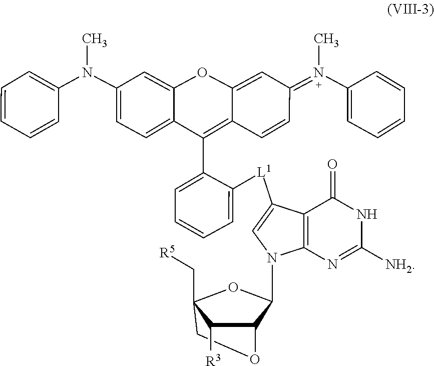

L¹, R³, and R⁵ are as described herein, including in embodiments.

In embodiments, the compound has the formula:

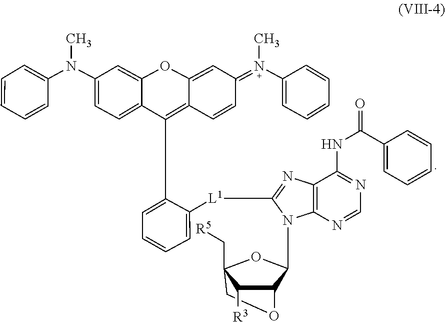

L¹, R³, and R⁵ are as described herein, including in embodiments.

In embodiments, the compound has the formula:

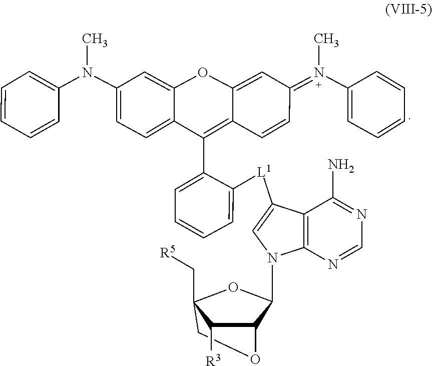

L¹, R³, and R⁵ are as described herein, including in embodiments.

In embodiments, the compound has the formula:

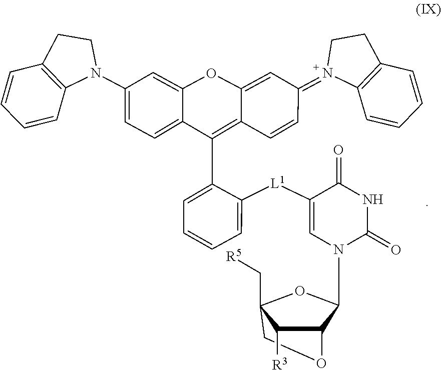

L¹, R³, and R⁵ are as described herein, including in embodiments.

In embodiments, the compound has the formula:

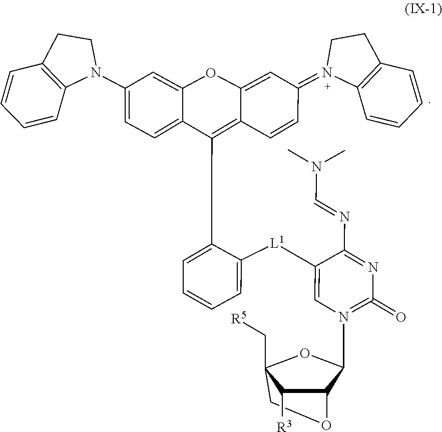

L¹, R³, and R⁵ are as described herein, including in embodiments.

In embodiments, the compound has the formula:

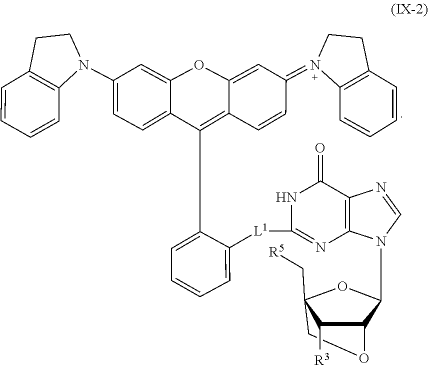

L¹, R³, and R⁵ are as described herein, including in embodiments.

In embodiments, the compound has the formula:

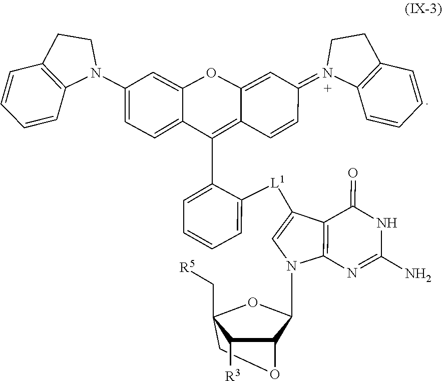

L¹, R³, and R⁵ are as described herein, including in embodiments.

In embodiments, the compound has the formula:

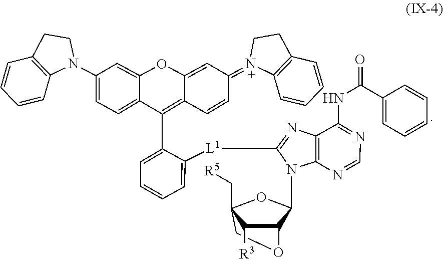

L¹, R³, and R⁵ are as described herein, including in embodiments.

In embodiments, the compound has the formula:

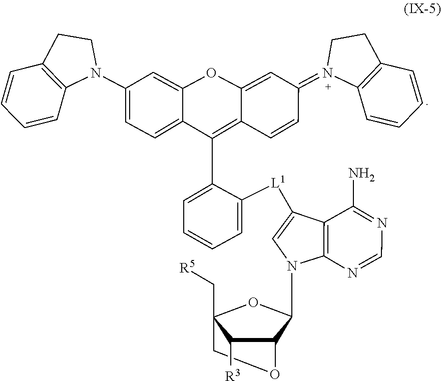

L¹, R³, and R⁵ are as described herein, including in embodiments.

In embodiments, L¹ is a divalent linker including 4 to 30 atoms.

In embodiments, L¹ is L¹⁰¹-L¹⁰²-L¹⁰³-L¹⁰⁴-L¹⁰⁵.

L¹⁰¹, L¹⁰², L¹⁰³, L¹⁰⁴, and L¹⁰⁵ are independently a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L¹⁰¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰¹ is substituted with a plurality of groups selected from substituent groups, size-limited substituent groups, and lower substituent groups; each substituent group, size-limited substituent group, and/or lower substituent group may optionally be different. In embodiments, when L¹⁰¹ is substituted, it is substituted with at least one substituent group. In embodiments, when L¹⁰¹ is substituted, it is substituted with at least one size-limited substituent group. In embodiments, when L¹⁰¹ is substituted, it is substituted with at least one lower substituent group.

In embodiments, L¹⁰¹ is a bond. In embodiments, L¹⁰¹ is —NH—. In embodiments, L¹⁰¹ is —O—. In embodiments, L¹⁰¹ is —S—. In embodiments, L¹⁰¹ is —S(O)—. In embodiments, L¹⁰¹ is —S(O)₂—. In embodiments, L¹⁰¹ is —C(O)—. In embodiments, L¹⁰¹ is —C(O)NH—. In embodiments, L¹⁰¹ is —NHC(O)—. In embodiments, L¹⁰¹ is —NHC(O)NH—. In embodiments, L¹⁰¹ is —C(O)O—. In embodiments, L¹⁰¹ is —OC(O)—. In embodiments, L¹⁰¹ is substituted or unsubstituted C₁-C₄ alkylene. In embodiments, L¹⁰¹ is substituted or unsubstituted 2 to 6 membered heteroalkylene.

In embodiments, a substituted L¹⁰² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰² is substituted with a plurality of groups selected from substituent groups, size-limited substituent groups, and lower substituent groups; each substituent group, size-limited substituent group, and/or lower substituent group may optionally be different. In embodiments, when L¹⁰² is substituted, it is substituted with at least one substituent group. In embodiments, when L¹⁰² is substituted, it is substituted with at least one size-limited substituent group. In embodiments, when L¹⁰² is substituted, it is substituted with at least one lower substituent group.

In embodiments, L¹⁰² is a bond. In embodiments, L¹⁰² is —NH—. In embodiments, L¹⁰² is —O—. In embodiments, L¹⁰² is —S—. In embodiments, L¹⁰² is —S(O)—. In embodiments, L¹⁰² is —S(O)₂—. In embodiments, L¹⁰² is —C(O)—. In embodiments, L¹⁰² is —C(O)NH—. In embodiments, L¹⁰² is —NHC(O)—. In embodiments, L¹⁰² is —NHC(O)NH—. In embodiments, L¹⁰² is —C(O)O—. In embodiments, L¹⁰² is —OC(O)—. In embodiments, L¹⁰² is substituted or unsubstituted C₁-C₄ alkylene. In embodiments, L¹⁰² is substituted or unsubstituted 2 to 6 membered heteroalkylene. In embodiments, L¹⁰² is an unsubstituted 3 to 8 membered heterocycloalkyl. In embodiments, L¹⁰² is an unsubstituted piperidinyl. In embodiments, L¹⁰² is

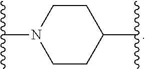 [SMILES: *C1CCN(*)CC1]

In embodiments, a substituted L¹⁰³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰³ is substituted with a plurality of groups selected from substituent groups, size-limited substituent groups, and lower substituent groups; each substituent group, size-limited substituent group, and/or lower substituent group may optionally be different. In embodiments, when L¹⁰³ is substituted, it is substituted with at least one substituent group. In embodiments, when L¹⁰³ is substituted, it is substituted with at least one size-limited substituent group. In embodiments, when L¹⁰³ is substituted, it is substituted with at least one lower substituent group.

In embodiments, L¹⁰³ is a bond. In embodiments, L¹⁰³ is —NH—. In embodiments, L¹⁰³ is —O—. In embodiments, L¹⁰³ is —S—. In embodiments, L¹⁰³ is —S(O)—. In embodiments, L¹⁰³ is —S(O)₂—. In embodiments, L¹⁰³ is —C(O)—. In embodiments, L¹⁰³ is —C(O)NH—. In embodiments, L¹⁰³ is —NHC(O)—. In embodiments, L¹⁰³ is —NHC(O)NH—. In embodiments, L¹⁰³ is —C(O)O—. In embodiments, L¹⁰³ is —OC(O)—. In embodiments, L¹⁰³ is substituted or unsubstituted C₁-C₄ alkylene. In embodiments, L¹⁰³ is substituted or unsubstituted 2 to 6 membered heteroalkylene.

In embodiments, a substituted L¹⁰⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰⁴ is substituted with a plurality of groups selected from substituent groups, size-limited substituent groups, and lower substituent groups; each substituent group, size-limited substituent group, and/or lower substituent group may optionally be different. In embodiments, when L¹⁰⁴ is substituted, it is substituted with at least one substituent group. In embodiments, when L¹⁰⁴ is substituted, it is substituted with at least one size-limited substituent group. In embodiments, when L¹⁰⁴ is substituted, it is substituted with at least one lower substituent group.

In embodiments, L¹⁰⁴ is a bond. In embodiments, L¹⁰⁴ is —NH—. In embodiments, L¹⁰⁴ is —O—. In embodiments, L¹⁰⁴ is —S—. In embodiments, L¹⁰⁴ is —S(O)—. In embodiments, L¹⁰⁴ is —S(O)₂—. In embodiments, L¹⁰⁴ is —C(O)—. In embodiments, L¹⁰⁴ is —C(O)NH—. In embodiments, L¹⁰⁴ is —NHC(O)—. In embodiments, L¹⁰⁴ is —NHC(O)NH—. In embodiments, L¹⁰⁴ is —C(O)O—. In embodiments, L¹⁰⁴ is —OC(O)—. In embodiments, L¹⁰⁴ is an unsubstituted C₁-C₁₀ alkylene. In embodiments, L¹⁰⁴ is an unsubstituted methylene. In embodiments, L¹⁰⁴ is an unsubstituted ethylene. In embodiments, L¹⁰⁴ is an unsubstituted propylene. In embodiments, L¹⁰⁴ is an unsubstituted n-propylene. In embodiments, L¹⁰⁴ is an unsubstituted butylene. In embodiments, L¹⁰⁴ is an unsubstituted n-butylene. In embodiments, L¹⁰⁴ is an unsubstituted pentylene. In embodiments, L¹⁰⁴ is an unsubstituted n-pentylene. In embodiments, L¹⁰⁴ is an unsubstituted hexylene. In embodiments, L¹⁰⁴ is an unsubstituted n-hexylene. In embodiments, L¹⁰⁴ is an unsubstituted heptylene. In embodiments, L¹⁰⁴ is an unsubstituted n-heptylene. In embodiments, L¹⁰⁴ is an unsubstituted octylene. In embodiments, L¹⁰⁴ is an unsubstituted n-octylene. In embodiments, L¹⁰⁴ is an unsubstituted C₂-C₆ alkynylene. In embodiments, L¹⁰⁴ is an unsubstituted ethynylene. In embodiments, L¹⁰⁴ is an unsubstituted propynylene. In embodiments, L¹⁰⁴ is an unsubstituted butynylene. In embodiments, L¹⁰⁴ is an unsubstituted pentynylene. In embodiments, L¹⁰⁴ is an unsubstituted hexynylene. In embodiments, L¹⁰⁴ is

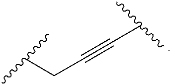 [SMILES: *C#CC*]

In embodiments, L¹⁰⁴ is a substituted or unsubstituted 2 to 6 membered heteroalkylene. In embodiments, L¹⁰⁴ is an unsubstituted 2 to 6 membered heteroalkylene. In embodiments, L¹⁰⁴ is

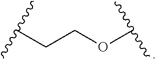 [SMILES: *CCO*]

In embodiments, L¹⁰⁴ is

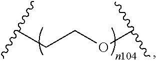

wherein n104 is an integer from 1 to 10. In embodiments, n104 is 1. In embodiments, n104 is 2. In embodiments, n104 is 3. In embodiments, n104 is 4. In embodiments, n104 is 5. In embodiments, n104 is 6. In embodiments, n104 is 7. In embodiments, n104 is 8. In embodiments, n104 is 9. In embodiments, n104 is 10. In embodiments, L¹⁰⁴ is substituted or unsubstituted phenylene. In embodiments, L⁴ is unsubstituted phenylene. In embodiments, L¹⁰⁴ is

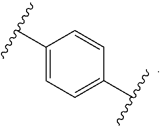 [SMILES: *c1ccc(*)cc1]

In embodiments, a substituted L¹⁰⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰⁵ is substituted with a plurality of groups selected from substituent groups, size-limited substituent groups, and lower substituent groups; each substituent group, size-limited substituent group, and/or lower substituent group may optionally be different. In embodiments, when L¹⁰⁵ is substituted, it is substituted with at least one substituent group. In embodiments, when L¹⁰⁵ is substituted, it is substituted with at least one size-limited substituent group. In embodiments, when L¹⁰⁵ is substituted, it is substituted with at least one lower substituent group.

In embodiments, L¹⁰⁵ is a bond. In embodiments, L¹⁰⁵ is —NH—. In embodiments, L¹⁰⁵ is —O—. In embodiments, L¹⁰⁵ is —S—. In embodiments, L¹⁰⁵ is —S(O)—. In embodiments, L¹⁰⁵ is —S(O)₂—. In embodiments, L¹⁰⁵ is —C(O)—. In embodiments, L¹⁰⁵ is —C(O)NH—. In embodiments, L¹⁰⁵ is —NHC(O)—. In embodiments, L¹⁰⁵ is —NHC(O)NH—. In embodiments, L¹⁰⁵ is —C(O)O—. In embodiments, L¹⁰⁵ is —OC(O)—. In embodiments, L¹⁰⁵ is an unsubstituted C₁-C₁₀ alkylene. In embodiments, L¹⁰⁵ is substituted or unsubstituted C₁-C₄ alkylene. In embodiments, L¹⁰⁵ is an unsubstituted methylene. In embodiments, L¹⁰⁵ is an unsubstituted ethylene. In embodiments, L¹⁰⁵ is an unsubstituted propylene. In embodiments, L¹⁰⁵ is an unsubstituted n-propylene. In embodiments, L¹⁰⁵ is an unsubstituted butylene. In embodiments, L¹⁰⁵ is an unsubstituted n-butylene. In embodiments, L¹⁰⁵ is an unsubstituted pentylene. In embodiments, L¹⁰⁵ is an unsubstituted n-pentylene. In embodiments, L¹⁰⁵ is an unsubstituted hexylene. In embodiments, L¹⁰⁵ is an unsubstituted n-hexylene. In embodiments, L¹⁰⁵ is an unsubstituted heptylene. In embodiments, L¹⁰⁵ is an unsubstituted n-heptylene. In embodiments, L¹⁰⁵ is an unsubstituted octylene. In embodiments, L¹⁰⁵ is an unsubstituted n-octylene. In embodiments, L¹⁰⁵ is an unsubstituted C₂-C₆ alkynylene. In embodiments, L¹⁰⁵ is an unsubstituted ethynylene. In embodiments, L¹⁰⁵ is an unsubstituted propynylene. In embodiments, L¹⁰⁵ is an unsubstituted butynylene. In embodiments, L¹⁰⁵ is an unsubstituted pentynylene. In embodiments, L¹⁰⁵ is an unsubstituted hexynylene. In embodiments, L¹⁰⁵ is

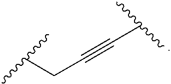 [SMILES: *C#CC*]

In embodiments, L¹⁰⁵ is a substituted 2 to 8 membered heteroalkylene. In embodiments, L¹⁰⁵ is an oxo-substituted 2 to 8 membered heteroalkylene. In embodiments, L¹⁰⁵ is an oxo-substituted 2 to 8 membered heteroalkenylene. In embodiments, L¹⁰⁵ is

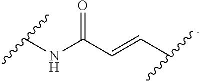 [SMILES: */C=C/C(=O)N*]

In embodiments, L¹⁰⁵ is

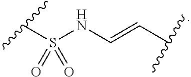 [SMILES: */C=C/NS(*)(=O)=O]

In embodiments, L¹⁰⁵ is

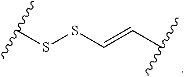 [SMILES: */C=C/SS*]

In embodiments, L¹⁰⁵ is a substituted or unsubstituted 2 to 8 membered heteroalkynylene. In embodiments, L¹⁰⁵ is

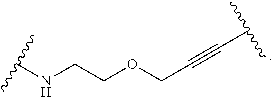 [SMILES: *C#CCOCCN*]

In embodiments, L¹⁰⁵ is

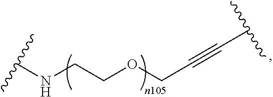

wherein n105 is an integer from 1 to 10. In embodiments, n105 is 1. In embodiments, n105 is 2. In embodiments, n105 is 3. In embodiments, n105 is 4. In embodiments, n105 is 5. In embodiments, n105 is 6. In embodiments, n105 is 7. In embodiments, n105 is 8. In embodiments, n105 is 9. In embodiments, n105 is 10. In embodiments, L¹⁰⁵ is an unsubstituted 5 to 10 membered heteroarylene. In embodiments, L¹⁰⁵ is an unsubstituted triazolylene. In embodiments, L¹⁰⁵ is

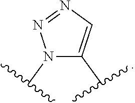 [SMILES: *c1cnnn1*]

In embodiments, L¹ is

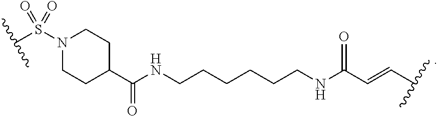 [SMILES: */C=C/C(=O)NCCCCCCNC(=O)C1CCN(S(*)(=O)=O)CC1]

In embodiments, L¹ is

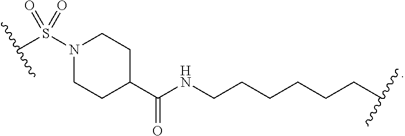 [SMILES: *CCCCCCNC(=O)C1CCN(S(*)(=O)=O)CC1]

In embodiments, L¹ is

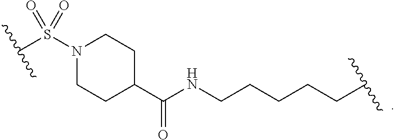 [SMILES: *CCCCCNC(=O)C1CCN(S(*)(=O)=O)CC1]

In embodiments, L¹ is

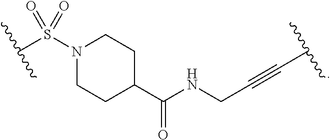 [SMILES: *C#CCNC(=O)C1CCN(S(*)(=O)=O)CC1]

In embodiments, L¹ is

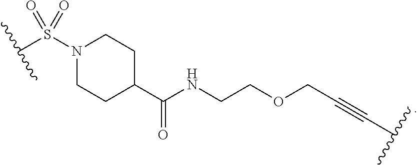 [SMILES: *C#CCOCCNC(=O)C1CCN(S(*)(=O)=O)CC1]

In embodiments, L¹ is

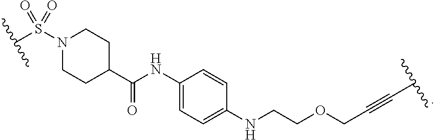 [SMILES: *C#CCOCCNc1ccc(NC(=O)C2CCN(S(*)(=O)=O)CC2)cc1]

In embodiments, L¹ is

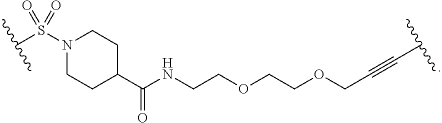 [SMILES: *C#CCOCCOCCNC(=O)C1CCN(S(*)(=O)=O)CC1]

In embodiments, L¹ is

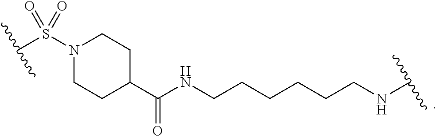 [SMILES: *NCCCCCCNC(=O)C1CCN(S(*)(=O)=O)CC1]

In embodiments, L¹ is

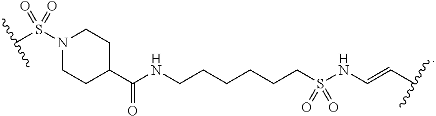 [SMILES: */C=C/NS(=O)(=O)CCCCCCNC(=O)C1CCN(S(*)(=O)=O)CC1]

In embodiments, L¹ is

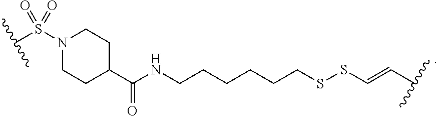 [SMILES: */C=C/SSCCCCCCNC(=O)C1CCN(S(*)(=O)=O)CC1]

In embodiments, L¹ is

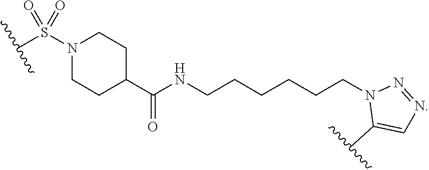 [SMILES: *c1cnnn1CCCCCCNC(=O)C1CCN(S(*)(=O)=O)CC1]

In embodiments, a substituted R¹ (e.g., substituted alkyl and/or substituted heteroalkyl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

In embodiments, R¹ is hydrogen. In embodiments, R¹ is unsubstituted C₁-C₄ alkyl. In embodiments, R¹ is unsubstituted methyl. In embodiments, R¹ is unsubstituted ethyl. In embodiments, R¹ is unsubstituted propyl. In embodiments, R¹ is unsubstituted n-propyl. In embodiments, R¹ is unsubstituted isopropyl. In embodiments, R¹ is unsubstituted butyl. In embodiments, R¹ is unsubstituted n-butyl. In embodiments, R¹ is unsubstituted isobutyl. In embodiments, R¹ is unsubstituted tert-butyl.

In embodiments, R² is hydrogen or —OH. In embodiments, R² is hydrogen. In embodiments, R² is —OR^{2A}. In embodiments, R² is —OH.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, R^{2A} is hydrogen. In embodiments, R^{2A} is unsubstituted C₁-C₄ alkyl. In embodiments, R^{2A} is unsubstituted methyl. In embodiments, R^{2A} is unsubstituted ethyl. In embodiments, R^{2A} is unsubstituted propyl. In embodiments, R^{2A} is unsubstituted n-propyl. In embodiments, R^{2A} is unsubstituted isopropyl. In embodiments, R^{2A} is unsubstituted butyl. In embodiments, R^{2A} is unsubstituted n-butyl. In embodiments, R^{2A} is unsubstituted isobutyl. In embodiments, R^{2A} is unsubstituted tert-butyl.

In embodiments, R³ is —OR^{3A}. In embodiments, R³ is —OH. In embodiments, R³ is —O—P(NR^{3B}R^{3C})—OR^{3A}. In embodiments, R³ is

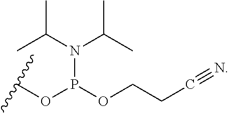 [SMILES: *OP(OCCC#N)N(C(C)C)C(C)C]

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, R^{3A} is hydrogen. In embodiments, R^{3A} is unsubstituted C₁-C₄ alkyl. In embodiments, R^{3A} is unsubstituted methyl. In embodiments, R^{3A} is unsubstituted ethyl. In embodiments, R^{3A} is unsubstituted propyl. In embodiments, R^{3A} is unsubstituted n-propyl. In embodiments, R^{3A} is unsubstituted isopropyl. In embodiments, R^{3A} is unsubstituted butyl. In embodiments, R^{3A} is unsubstituted n-butyl. In embodiments, R^{3A} is unsubstituted isobutyl. In embodiments, R^{3A} is unsubstituted tert-butyl. In embodiments, R^{3A} is substituted C₁-C₄ alkyl. In embodiments, R^{3A} is cyano-substituted C₁-C₄ alkyl. In embodiments, R^{3A} is

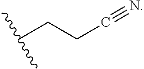 [SMILES: *CCC#N]

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, R^{3B} is hydrogen. In embodiments, R^{3B} is unsubstituted C₁-C₄ alkyl. In embodiments, R^{3B} is unsubstituted methyl. In embodiments, R^{3B} is unsubstituted ethyl. In embodiments, R^{3B} is unsubstituted propyl. In embodiments, R^{3B} is unsubstituted n-propyl. In embodiments, R^{3B} is unsubstituted isopropyl. In embodiments, R^{3B} is unsubstituted butyl. In embodiments, R^{3B} is unsubstituted n-butyl. In embodiments, R^{3B} is unsubstituted isobutyl. In embodiments, R^{3B} is unsubstituted tert-butyl.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, R^{3C} is hydrogen. In embodiments, R^{3C} is unsubstituted C₁-C₄ alkyl. In embodiments, R^{3C} is unsubstituted methyl. In embodiments, R^{3C} is unsubstituted ethyl. In embodiments, R^{3C} is unsubstituted propyl. In embodiments, R^{3C} is unsubstituted n-propyl. In embodiments, R^{3C} is unsubstituted isopropyl. In embodiments, R^{3C} is unsubstituted butyl. In embodiments, R^{3C} is unsubstituted n-butyl. In embodiments, R^{3C} is unsubstituted isobutyl. In embodiments, R^{3C} is unsubstituted tert-butyl.

In embodiments, R⁴ is hydrogen. In embodiments, R⁴ is unsubstituted methyl.

In embodiments, a substituted ring formed when R² and R⁴ substituents are joined (e.g., substituted heterocycloalkyl) is substituted with at least one substituent group, size-limited substituent group, or lower substituent group; wherein if the substituted ring formed when R² and R⁴ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R² and R⁴ substituents are joined is substituted, it is substituted with at least one substituent group. In embodiments, when the substituted ring formed when R² and R⁴ substituents are joined is substituted, it is substituted with at least one size-limited substituent group. In embodiments, when the substituted ring formed when R² and R⁴ substituents are joined is substituted, it is substituted with at least one lower substituent group.

In embodiments, R² and R⁴ substituents are joined to form a substituted or unsubstituted 3 to 8 membered heterocycloalkyl. In embodiments, R² and R⁴ substituents are joined to form a substituted or unsubstituted tetrahydrofuranyl.

In embodiments, R⁵ is —OH.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, R^{5A} is hydrogen or substituted C₁-C₆ alkyl. In embodiments, R^{5A} is hydrogen. In embodiments, R^{5A} is unsubstituted C₁-C₄ alkyl. In embodiments, R^{5A} is unsubstituted methyl. In embodiments, R^{5A} is unsubstituted ethyl. In embodiments, R^{5A} is unsubstituted propyl. In embodiments, R^{5A} is unsubstituted n-propyl. In embodiments, R^{5A} is unsubstituted isopropyl. In embodiments, R^{5A} is unsubstituted butyl. In embodiments, R^{5A} is unsubstituted n-butyl. In embodiments, R^{5A} is unsubstituted isobutyl. In embodiments, R^{5A} is unsubstituted tert-butyl. In embodiments, R^{5A} is substituted C₁-C₆ alkyl. In embodiments, R^{5A} is dimethoxytrityl. In embodiments, R^{5A} is

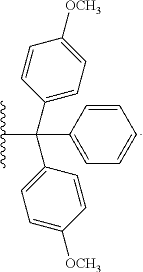 [SMILES: *C(c1ccccc1)(c1ccc(OC)cc1)c1ccc(OC)cc1]

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is hydrogen. In embodiments, R⁶ is halogen. In embodiments, R⁶ is —F. In embodiments, R⁶ is —Cl. In embodiments, R⁶ is —Br. In embodiments, R⁶ is —I. In embodiments, R⁶ is —CCl₃. In embodiments, R⁶ is —CBr₃. In embodiments, R⁶ is —CF₃. In embodiments, R⁶ is —CI₃. In embodiments, R⁶ is —CH₂Cl. In embodiments, R⁶ is —CH₂Br. In embodiments, R⁶ is —CH₂F. In embodiments, R⁶ is —CH₂I. In embodiments, R⁶ is —CHCl₂. In embodiments, R⁶ is —CHBr₂. In embodiments, R⁶ is —CHF₂. In embodiments, R⁶ is —CHI₂. In embodiments, R⁶ is —CN. In embodiments, R⁶ is —OH. In embodiments, R⁶ is —NH₂. In embodiments, R⁶ is —COOH. In embodiments, R⁶ is —CONH₂. In embodiments, R⁶ is —NO₂. In embodiments, R⁶ is —SH. In embodiments, R⁶ is —SO₃R^{A}. In embodiments, R⁶ is —SO₃H. In embodiments, R⁶ is —SO₂NH₂. In embodiments, R⁶ is —NHNH₂. In embodiments, R⁶ is —ONH₂. In embodiments, R⁶ is —NHC(O)NH₂. In embodiments, R⁶ is —NHSO₂H. In embodiments, R⁶ is —NHC(O)H. In embodiments, R⁶ is —NHC(O)OH. In embodiments, R⁶ is —NHOH. In embodiments, R⁶ is —OCCl₃. In embodiments, R⁶ is —OCBr₃. In embodiments, R⁶ is —OCF₃. In embodiments, R⁶ is —OCI₃. In embodiments, R⁶ is —OCH₂Cl. In embodiments, R⁶ is —OCH₂Br. In embodiments, R⁶ is —OCH₂F. In embodiments, R⁶ is —OCH₂I. In embodiments, R⁶ is —OCHCl₂. In embodiments, R⁶ is —OCHBr₂. In embodiments, R⁶ is —OCHF₂. In embodiments, R⁶ is —OCHI₂. In embodiments, R⁶ is —SF₅. In embodiments, R⁶ is —N₃. In embodiments, R⁶ is unsubstituted C₁-C₄ alkyl. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl. In embodiments, R⁶ is unsubstituted 2 to 6 membered heteroalkyl. In embodiments, R⁶ is unsubstituted methoxy. In embodiments, R⁶ is unsubstituted ethoxy. In embodiments, R⁶ is unsubstituted propoxy. In embodiments, R⁶ is unsubstituted n-propoxy. In embodiments, R⁶ is unsubstituted isopropoxy. In embodiments, R⁶ is unsubstituted butoxy.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁷ is hydrogen. In embodiments, R⁷ is halogen. In embodiments, R⁷ is —F. In embodiments, R⁷ is —Cl. In embodiments, R⁷ is —Br. In embodiments, R⁷ is —I. In embodiments, R⁷ is —CCl₃. In embodiments, R⁷ is —CBr₃. In embodiments, R⁷ is —CF₃. In embodiments, R⁷ is —CI₃. In embodiments, R⁷ is —CH₂Cl. In embodiments, R⁷ is —CH₂Br. In embodiments, R⁷ is —CH₂F. In embodiments, R⁷ is —CH₂I. In embodiments, R⁷ is —CHCl₂. In embodiments, R⁷ is —CHBr₂. In embodiments, R⁷ is —CHF₂. In embodiments, R⁷ is —CHI₂. In embodiments, R⁷ is —CN. In embodiments, R⁷ is —OH. In embodiments, R⁷ is —NH₂. In embodiments, R⁷ is —COOH. In embodiments, R⁷ is —CONH₂. In embodiments, R⁷ is —NO₂. In embodiments, R⁷ is —SH. In embodiments, R⁷ is —SO₃R^{A}. In embodiments, R⁷ is —SO₃H. In embodiments, R⁷ is —SO₂NH₂. In embodiments, R⁷ is —NHNH₂. In embodiments, R⁷ is —ONH₂. In embodiments, R⁷ is —NHC(O)NH₂. In embodiments, R⁷ is —NHSO₂H. In embodiments, R⁷ is —NHC(O)H. In embodiments, R⁷ is —NHC(O)OH. In embodiments, R⁷ is —NHOH. In embodiments, R⁷ is —OCCl₃. In embodiments, R⁷ is —OCBr₃. In embodiments, R⁷ is —OCF₃. In embodiments, R⁷ is —OCI₃. In embodiments, R⁷ is —OCH₂Cl. In embodiments, R⁷ is —OCH₂Br. In embodiments, R⁷ is —OCH₂F. In embodiments, R⁷ is —OCH₂I. In embodiments, R⁷ is —OCHCl₂. In embodiments, R⁷ is —OCHBr₂. In embodiments, R⁷ is —OCHF₂. In embodiments, R⁷ is —OCHI₂. In embodiments, R⁷ is —SF₅. In embodiments, R⁷ is —N₃. In embodiments, R⁷ is unsubstituted C₁-C₄ alkyl. In embodiments, R⁷ is unsubstituted methyl. In embodiments, R⁷ is unsubstituted ethyl. In embodiments, R⁷ is unsubstituted propyl. In embodiments, R⁷ is unsubstituted n-propyl. In embodiments, R⁷ is unsubstituted isopropyl. In embodiments, R⁷ is unsubstituted butyl. In embodiments, R⁷ is unsubstituted n-butyl. In embodiments, R⁷ is unsubstituted isobutyl. In embodiments, R⁷ is unsubstituted tert-butyl. In embodiments, R⁷ is unsubstituted 2 to 6 membered heteroalkyl. In embodiments, R⁷ is unsubstituted methoxy. In embodiments, R⁷ is unsubstituted ethoxy. In embodiments, R⁷ is unsubstituted propoxy. In embodiments, R⁷ is unsubstituted n-propoxy. In embodiments, R⁷ is unsubstituted isopropoxy. In embodiments, R⁷ is unsubstituted butoxy.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is hydrogen. In embodiments, R⁸ is halogen. In embodiments, R⁸ is —F. In embodiments, R⁸ is —Cl. In embodiments, R⁸ is —Br. In embodiments, R⁸ is —I. In embodiments, R⁸ is —CCl₃. In embodiments, R⁸ is —CBr₃. In embodiments, R⁸ is —CF₃. In embodiments, R⁸ is —CI₃. In embodiments, R⁸ is —CH₂Cl. In embodiments, R⁸ is —CH₂Br. In embodiments, R⁸ is —CH₂F. In embodiments, R⁸ is —CH₂I. In embodiments, R⁸ is —CHCl₂. In embodiments, R⁸ is —CHBr₂. In embodiments, R⁸ is —CHF₂. In embodiments, R⁸ is —CHI₂. In embodiments, R⁸ is —CN. In embodiments, R⁸ is —OH. In embodiments, R⁸ is —NH₂. In embodiments, R⁸ is —COOH. In embodiments, R⁸ is —CONH₂. In embodiments, R⁸ is —NO₂. In embodiments, R⁸ is —SH. In embodiments, R⁸ is —SO₃R^{A}. In embodiments, R⁸ is —SO₃H. In embodiments, R⁸ is —SO₂NH₂. In embodiments, R⁸ is —NHNH₂. In embodiments, R⁸ is —ONH₂. In embodiments, R⁸ is —NHC(O)NH₂. In embodiments, R⁸ is —NHSO₂H. In embodiments, R⁸ is —NHC(O)H. In embodiments, R⁸ is —NHC(O)OH. In embodiments, R⁸ is —NHOH. In embodiments, R⁸ is —OCCl₃. In embodiments, R⁸ is —OCBr₃. In embodiments, R⁸ is —OCF₃. In embodiments, R⁸ is —OCI₃. In embodiments, R⁸ is —OCH₂Cl. In embodiments, R⁸ is —OCH₂Br. In embodiments, R⁸ is —OCH₂F. In embodiments, R′ is —OCH₂I. In embodiments, R⁸ is —OCHCl₂. In embodiments, R⁸ is —OCHBr₂. In embodiments, R⁸ is —OCHF₂. In embodiments, R⁸ is —OCHI₂. In embodiments, R⁸ is —SF₅. In embodiments, R⁸ is —N₃. In embodiments, R⁸ is unsubstituted C₁-C₄ alkyl. In embodiments, R⁸ is unsubstituted methyl. In embodiments, R⁸ is unsubstituted ethyl. In embodiments, R⁸ is unsubstituted propyl. In embodiments, R⁸ is unsubstituted n-propyl. In embodiments, R⁸ is unsubstituted isopropyl. In embodiments, R⁸ is unsubstituted butyl. In embodiments, R⁸ is unsubstituted n-butyl. In embodiments, R⁸ is unsubstituted isobutyl. In embodiments, R⁸ is unsubstituted tert-butyl. In embodiments, R⁸ is unsubstituted 2 to 6 membered heteroalkyl. In embodiments, R⁸ is unsubstituted methoxy. In embodiments, R⁸ is unsubstituted ethoxy. In embodiments, R⁸ is unsubstituted propoxy. In embodiments, R⁸ is unsubstituted n-propoxy. In embodiments, R⁸ is unsubstituted isopropoxy. In embodiments, R⁸ is unsubstituted butoxy.

In embodiments, a substituted R⁹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is hydrogen. In embodiments, R⁹ is halogen. In embodiments, R⁹ is —F. In embodiments, R⁹ is —Cl. In embodiments, R⁹ is —Br. In embodiments, R⁹ is —I. In embodiments, R⁹ is —CCl₃. In embodiments, R⁹ is —CBr₃. In embodiments, R⁹ is —CF₃. In embodiments, R⁹ is —CI₃. In embodiments, R⁹ is —CH₂Cl. In embodiments, R⁹ is —CH₂Br. In embodiments, R⁹ is —CH₂F. In embodiments, R⁹ is —CH₂I. In embodiments, R⁹ is —CHCl₂. In embodiments, R⁹ is —CHBr₂. In embodiments, R⁹ is —CHF₂. In embodiments, R⁹ is —CHI₂. In embodiments, R⁹ is —CN. In embodiments, R⁹ is —OH. In embodiments, R⁹ is —NH₂. In embodiments, R⁹ is —COOH. In embodiments, R⁹ is —CONH₂. In embodiments, R⁹ is —NO₂. In embodiments, R⁹ is —SH. In embodiments, R⁹ is —SO₃R^{A}. In embodiments, R⁹ is —SO₃H. In embodiments, R⁹ is —SO₂NH₂. In embodiments, R⁹ is —NHNH₂. In embodiments, R⁹ is —ONH₂. In embodiments, R⁹ is —NHC(O)NH₂. In embodiments, R⁹ is —NHSO₂H. In embodiments, R⁹ is —NHC(O)H. In embodiments, R⁹ is —NHC(O)OH. In embodiments, R⁹ is —NHOH. In embodiments, R⁹ is —OCCl₃. In embodiments, R⁹ is —OCBr₃. In embodiments, R⁹ is —OCF₃. In embodiments, R⁹ is —OCI₃. In embodiments, R⁹ is —OCH₂Cl. In embodiments, R⁹ is —OCH₂Br. In embodiments, R⁹ is —OCH₂F. In embodiments, R⁹ is —OCH₂I. In embodiments, R⁹ is —OCHCl₂. In embodiments, R⁹ is —OCHBr₂. In embodiments, R⁹ is —OCHF₂. In embodiments, R⁹ is —OCHI₂. In embodiments, R⁹ is —SF₅. In embodiments, R⁹ is —N₃. In embodiments, R⁹ is unsubstituted C₁-C₄ alkyl. In embodiments, R⁹ is unsubstituted methyl. In embodiments, R⁹ is unsubstituted ethyl. In embodiments, R⁹ is unsubstituted propyl. In embodiments, R⁹ is unsubstituted n-propyl. In embodiments, R⁹ is unsubstituted isopropyl. In embodiments, R⁹ is unsubstituted butyl. In embodiments, R⁹ is unsubstituted n-butyl. In embodiments, R⁹ is unsubstituted isobutyl. In embodiments, R⁹ is unsubstituted tert-butyl. In embodiments, R⁹ is unsubstituted 2 to 6 membered heteroalkyl. In embodiments, R⁹ is unsubstituted methoxy. In embodiments, R⁹ is unsubstituted ethoxy. In embodiments, R⁹ is unsubstituted propoxy. In embodiments, R⁹ is unsubstituted n-propoxy. In embodiments, R⁹ is unsubstituted isopropoxy. In embodiments, R⁹ is unsubstituted butoxy.

In embodiments, a substituted R^{A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{A} is substituted with a plurality of groups selected from substituent groups, size-limited substituent groups, and lower substituent groups; each substituent group, size-limited substituent group, and/or lower substituent group may optionally be different. In embodiments, when R^{A} is substituted, it is substituted with at least one substituent group. In embodiments, when R^{A} is substituted, it is substituted with at least one size-limited substituent group. In embodiments, when R^{A} is substituted, it is substituted with at least one lower substituent group.

In embodiments, R^{A} is hydrogen. In embodiments, R^{A} is unsubstituted C₁-C₄ alkyl. In embodiments, R^{A} is unsubstituted methyl. In embodiments, R^{A} is unsubstituted ethyl. In embodiments, R^{A} is unsubstituted propyl. In embodiments, R^{A} is unsubstituted n-propyl. In embodiments, R^{A} is unsubstituted isopropyl. In embodiments, R^{A} is unsubstituted butyl. In embodiments, R^{A} is unsubstituted n-butyl. In embodiments, R^{A} is unsubstituted isobutyl. In embodiments, R^{A} is unsubstituted tert-butyl.

In embodiments, a substituted R¹⁰ (e.g., substituted alkyl and/or substituted heteroalkyl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, R¹⁰ is hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

In embodiments, R¹⁰ is hydrogen. In embodiments, R¹⁰ is unsubstituted C₁-C₄ alkyl. In embodiments, R¹⁰ is unsubstituted methyl. In embodiments, R¹⁰ is unsubstituted ethyl. In embodiments, R¹⁰ is unsubstituted propyl. In embodiments, R¹⁰ is unsubstituted n-propyl. In embodiments, R¹⁰ is unsubstituted isopropyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl.

In embodiments, a substituted ring formed when R¹ and R⁶ substituents are joined (e.g., substituted heterocycloalkyl and/or substituted heteroaryl) is substituted with at least one substituent group, size-limited substituent group, or lower substituent group; wherein if the substituted ring formed when R¹ and R⁶ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R¹ and R⁶ substituents are joined is substituted, it is substituted with at least one substituent group. In embodiments, when the substituted ring formed when R¹ and R⁶ substituents are joined is substituted, it is substituted with at least one size-limited substituent group. In embodiments, when the substituted ring formed when R¹ and R⁶ substituents are joined is substituted, it is substituted with at least one lower substituent group.

In embodiments, R¹ and R⁶ substituents are joined to form a substituted or unsubstituted 3 to 8 membered heterocycloalkyl. In embodiments, R¹ and R⁶ substituents are joined to form a substituted or unsubstituted pyrrolidinyl. In embodiments, R¹ and R⁶ substituents are joined to form an unsubstituted pyrrolidinyl. In embodiments, R¹ and R⁶ substituents are joined to form a substituted or unsubstituted 5 to 6 membered heteroaryl.

In embodiments, a substituted ring formed when R⁸ and R¹⁰ substituents are joined (e.g., substituted heterocycloalkyl and/or substituted heteroaryl) is substituted with at least one substituent group, size-limited substituent group, or lower substituent group; wherein if the substituted ring formed when R⁸ and R¹⁰ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R⁸ and R¹⁰ substituents are joined is substituted, it is substituted with at least one substituent group. In embodiments, when the substituted ring formed when R⁸ and R¹⁰ substituents are joined is substituted, it is substituted with at least one size-limited substituent group. In embodiments, when the substituted ring formed when R⁸ and R¹⁰ substituents are joined is substituted, it is substituted with at least one lower substituent group.

In embodiments, R⁸ and R¹⁰ substituents are joined to form a substituted or unsubstituted 3 to 8 membered heterocycloalkyl. In embodiments, R⁸ and R¹⁰ substituents are joined to form a substituted or unsubstituted pyrrolidinyl. In embodiments, R⁸ and R¹⁰ substituents are joined to form an unsubstituted pyrrolidinyl. In embodiments, R⁸ and R¹⁰ substituents are joined to form a substituted or unsubstituted 5 to 6 membered heteroaryl.

In embodiments, the compound has the formula:

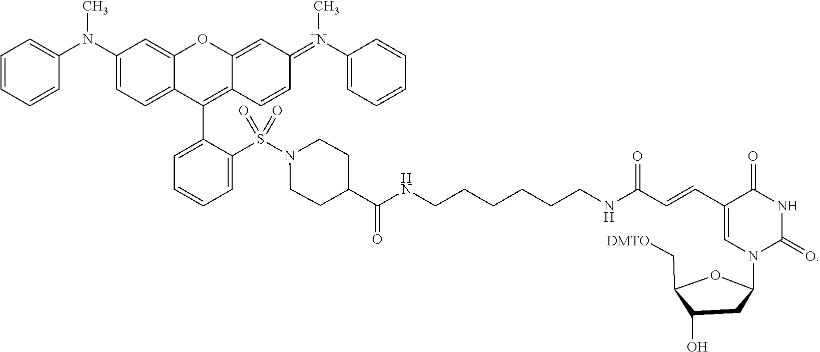

In embodiments, the compound has the formula:

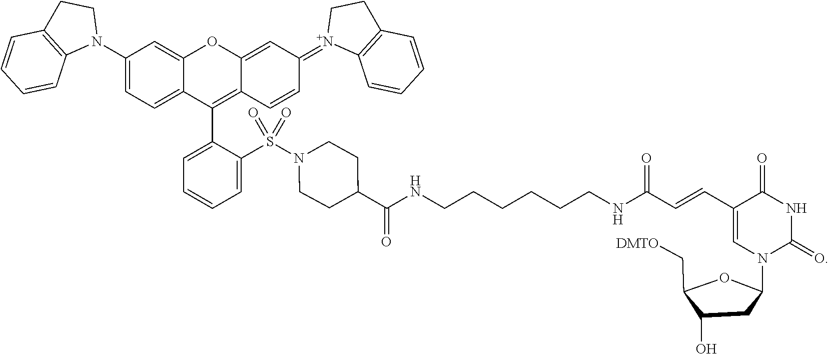

In embodiments, the compound has the formula:

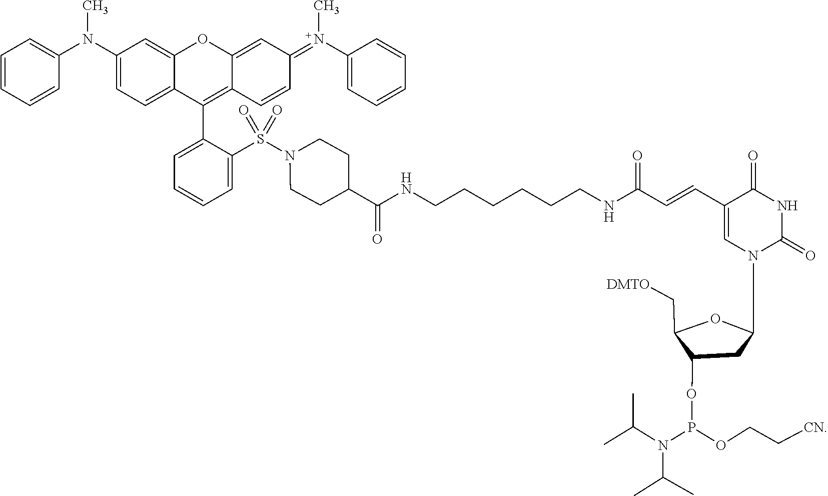

In embodiments, the compound has the formula:

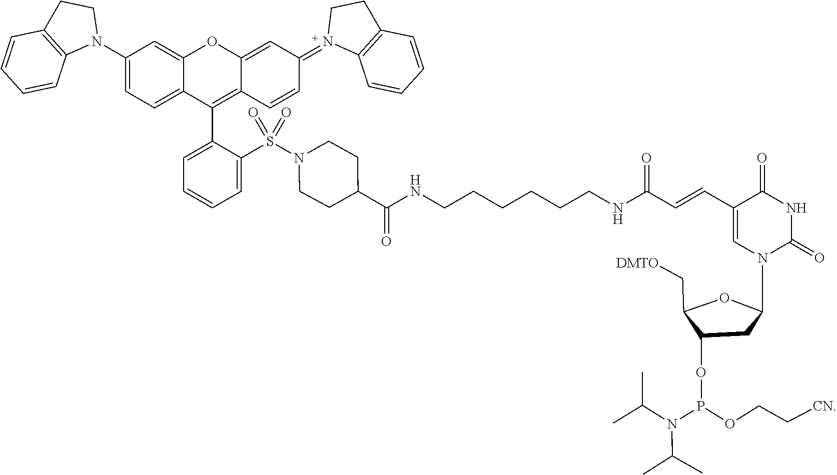

In embodiments, the compound has the formula:

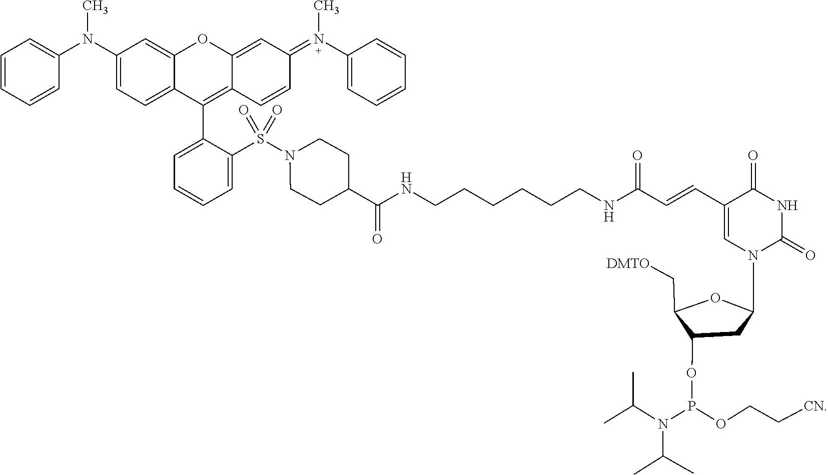

In embodiments, the compound has the formula:

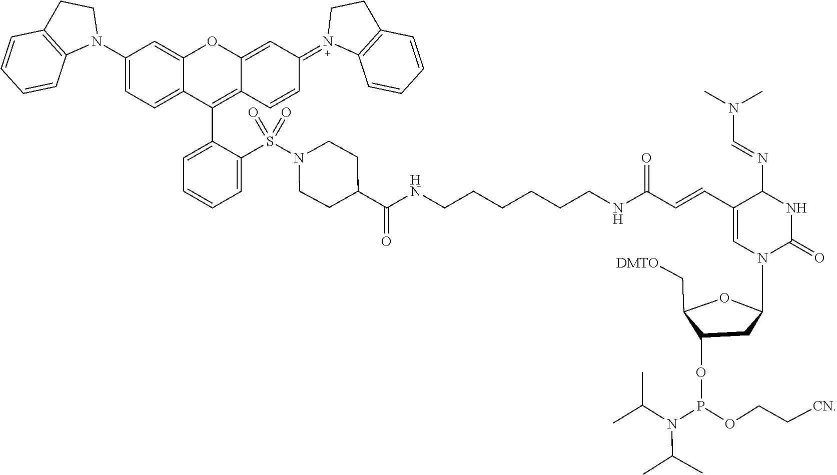

In embodiments, the compound has the formula:

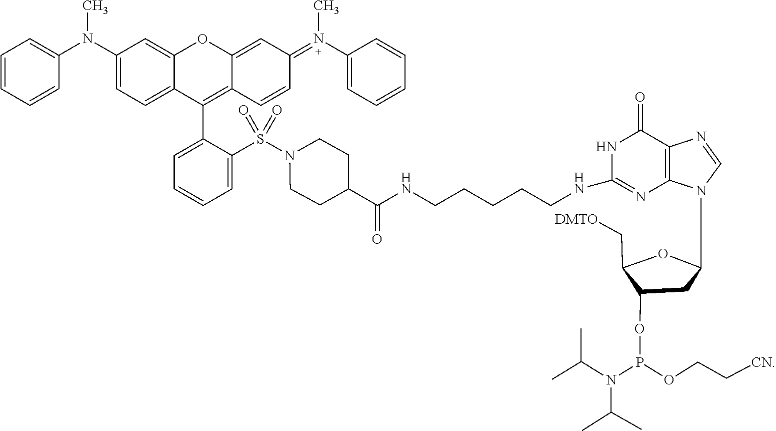

In embodiments, the compound has the formula:

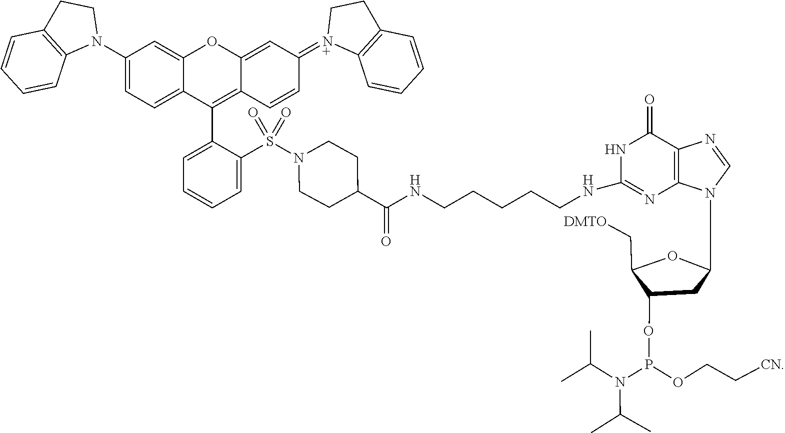

In embodiments, the compound has the formula:

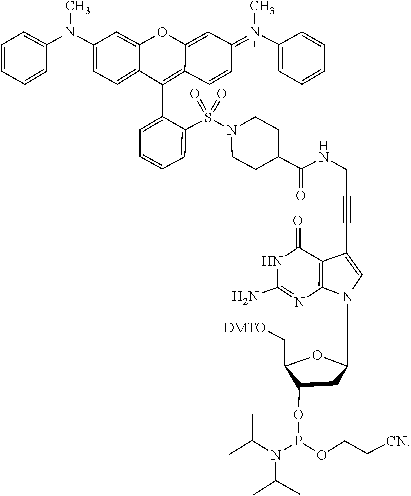

In embodiments, the compound has the formula:

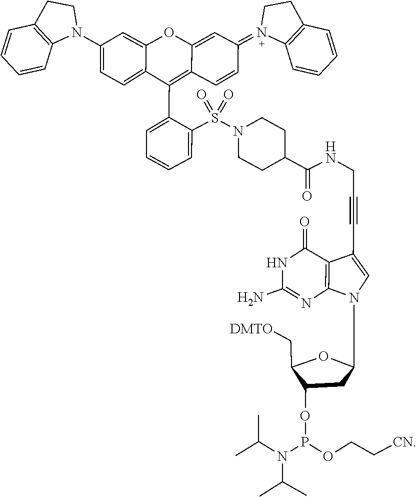

In embodiments, the compound has the formula:

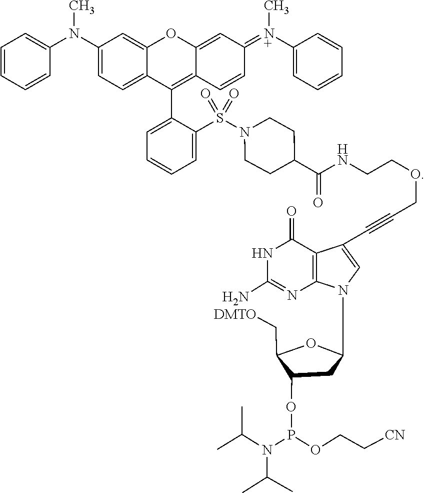

In embodiments, the compound has the formula:

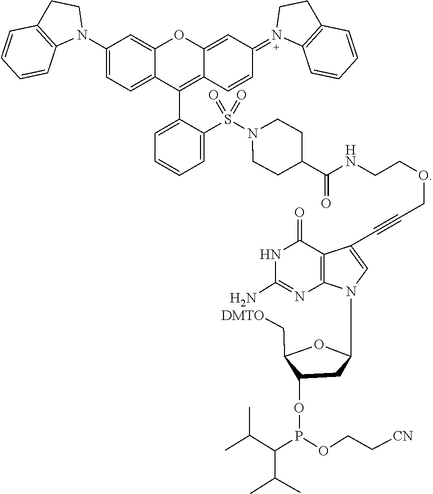

In embodiments, the compound has the formula:

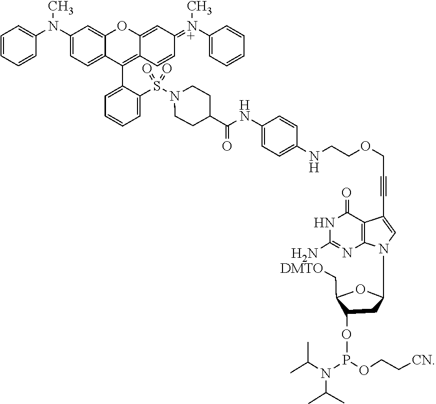

In embodiments, the compound has the formula:

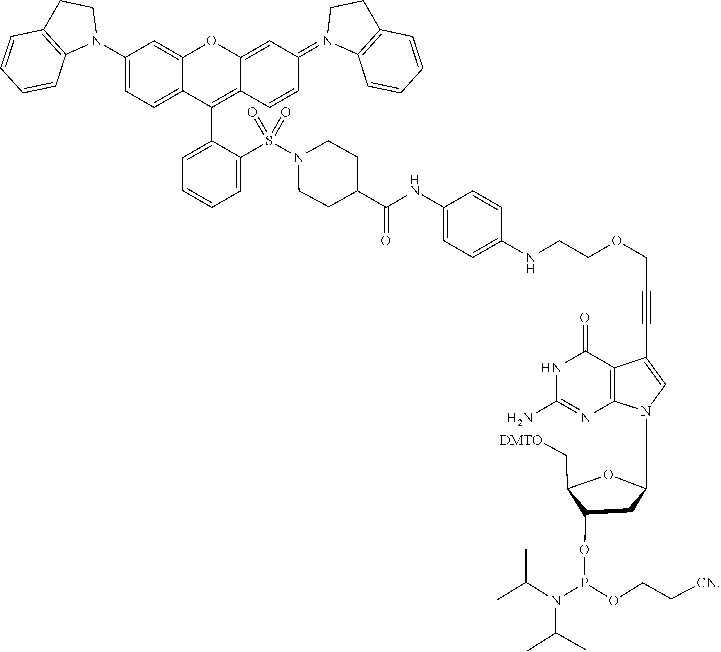

In embodiments, the compound has the formula:

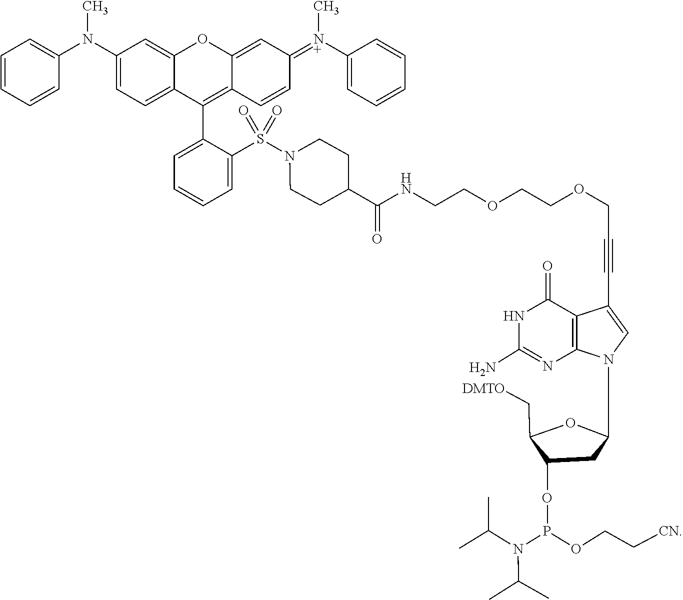

In embodiments, the compound has the formula:

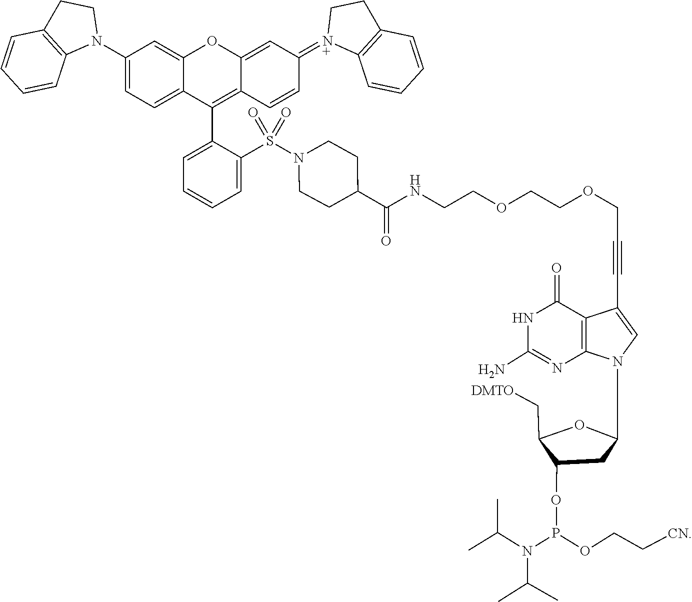

In embodiments, the compound has the formula:

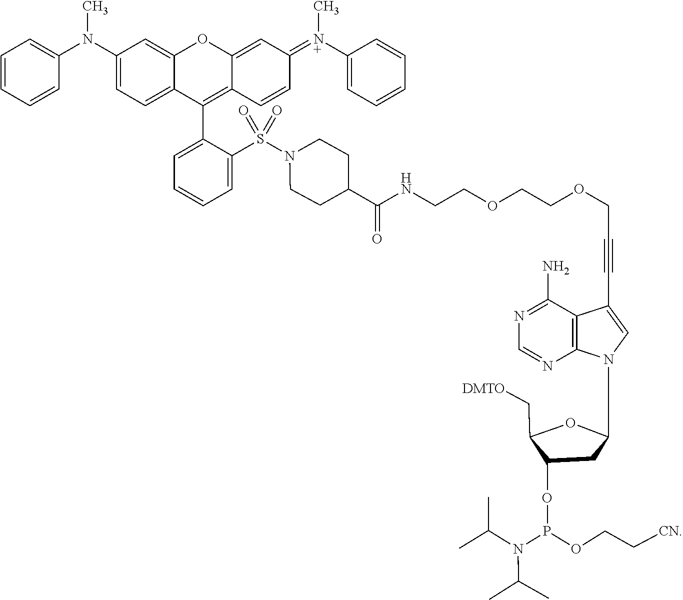

In embodiments, the compound has the formula:

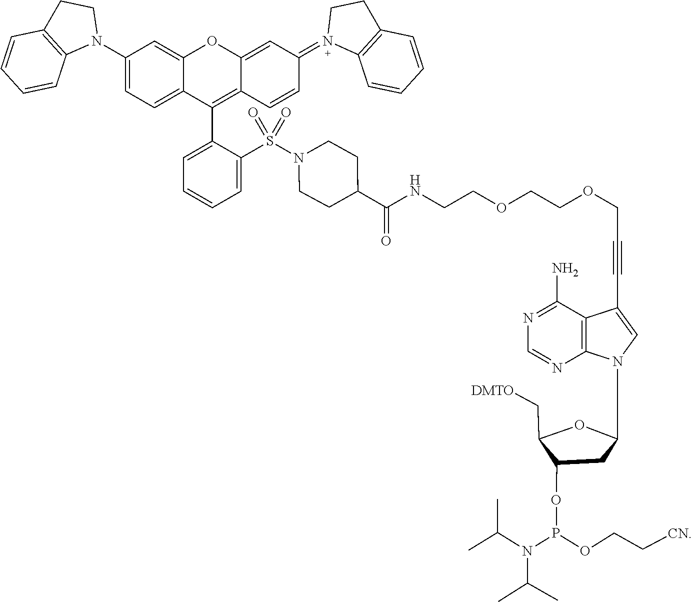

In embodiments, the compound has the formula:

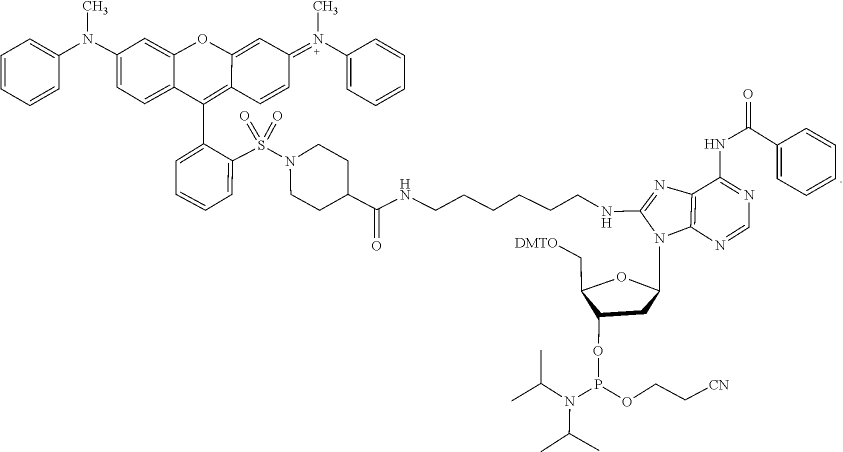

In embodiments, the compound has the formula:

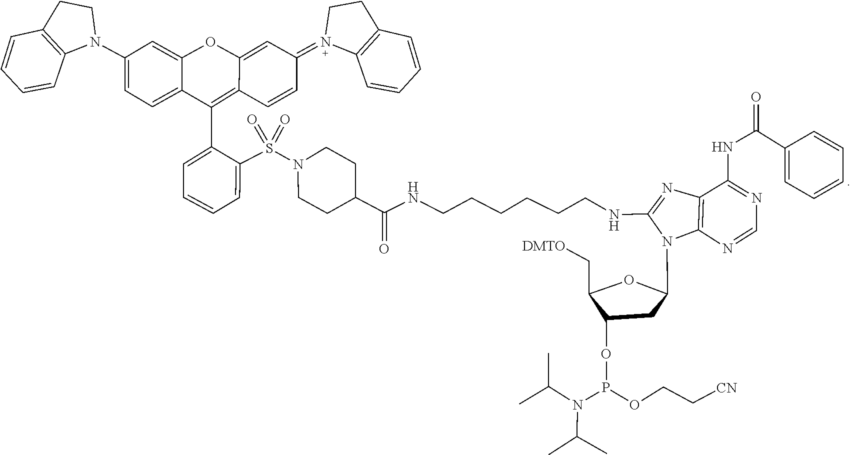

In embodiments, the compound has the formula:

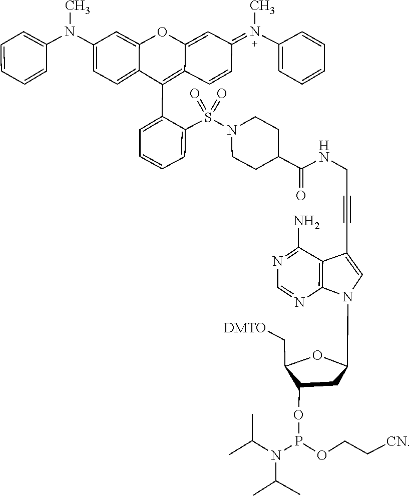

In embodiments, the compound has the formula:

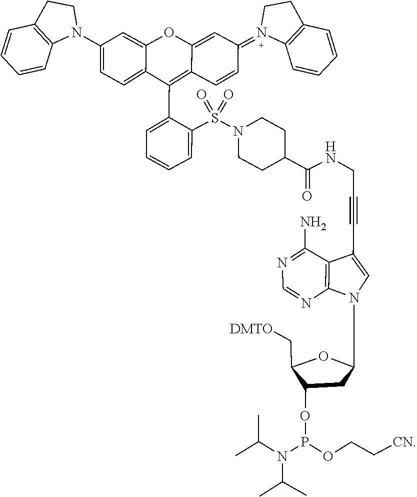

In embodiments, the compound has the formula:

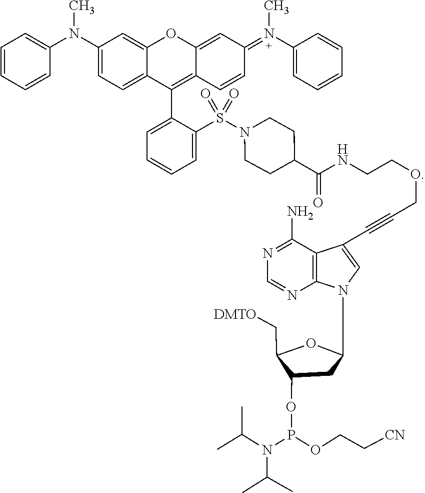

In embodiments, the compound has the formula:

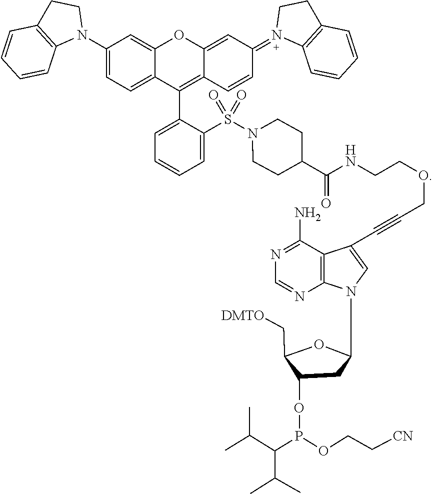

In embodiments, the compound has the formula:

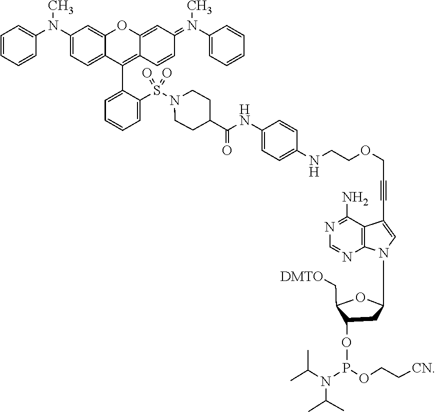

In embodiments, the compound has the formula:

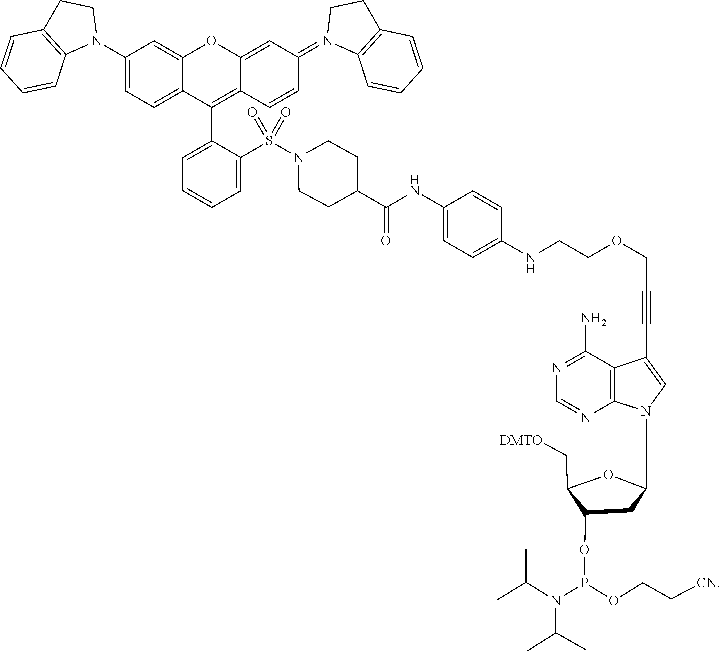

In embodiments, the compound has the formula:

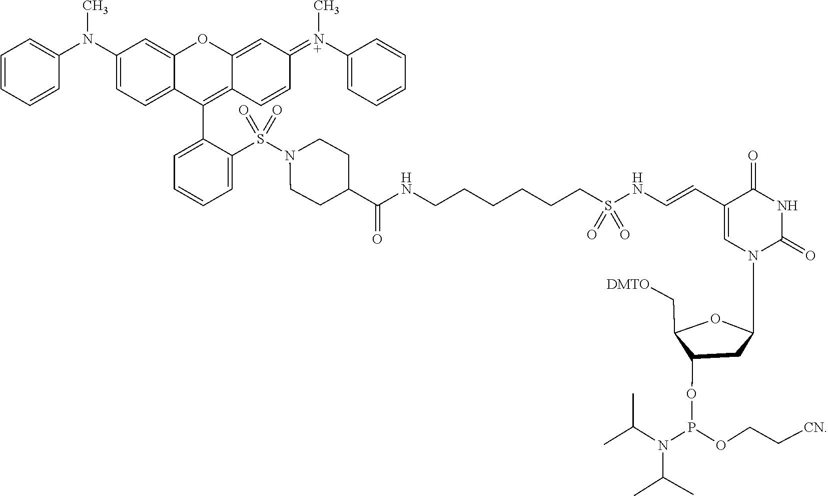

In embodiments, the compound has the formula:

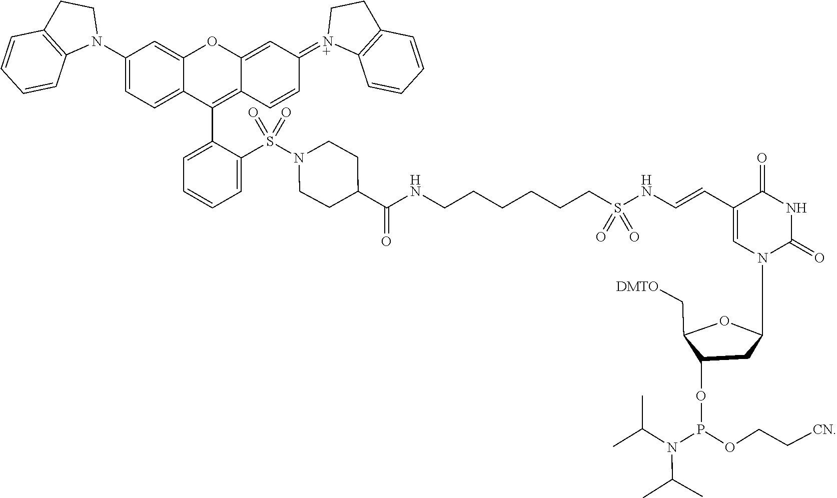

In embodiments, the compound has the formula:

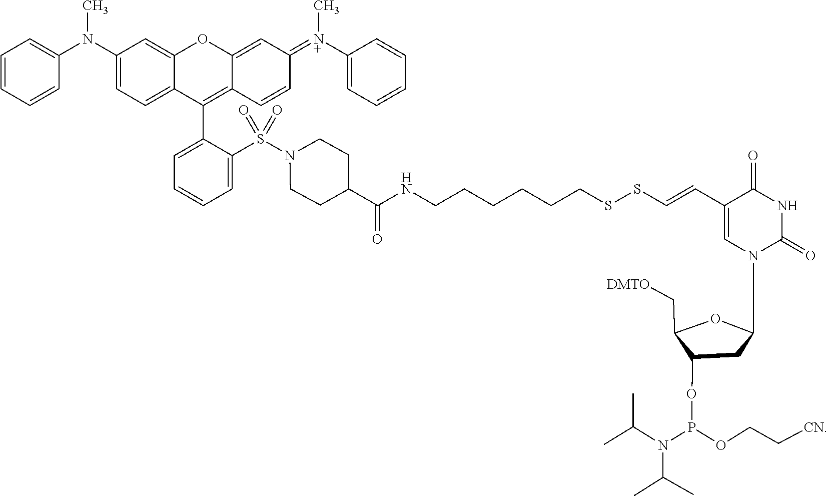

In embodiments, the compound has the formula:

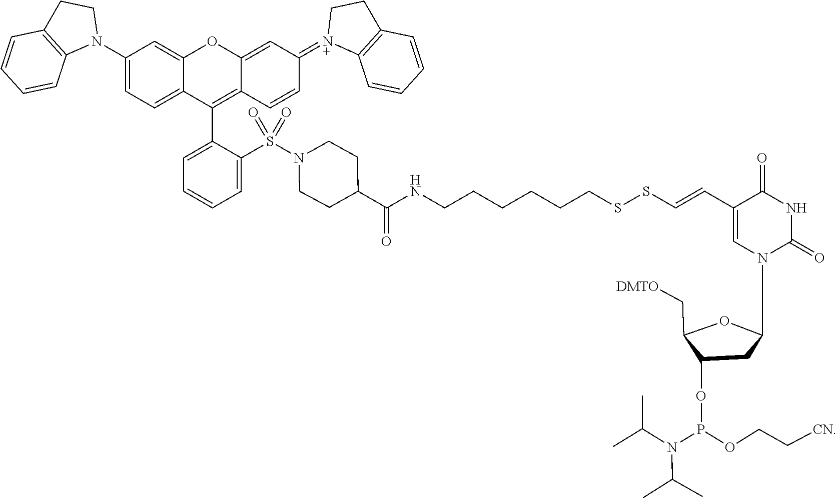

In embodiments, the compound has the formula:

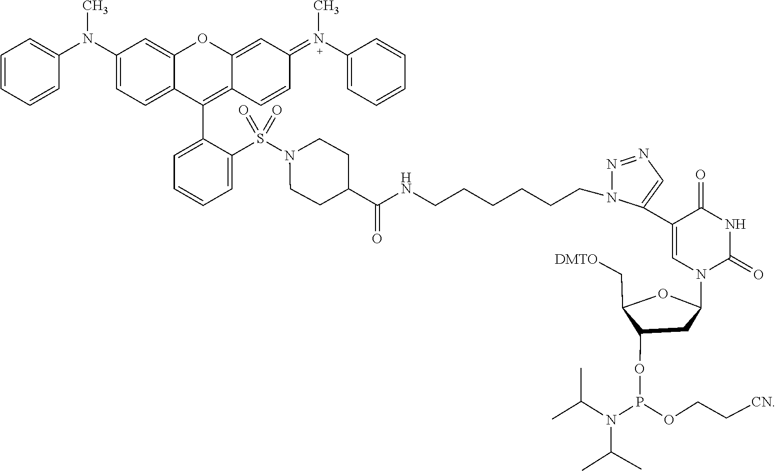

In embodiments, the compound has the formula:

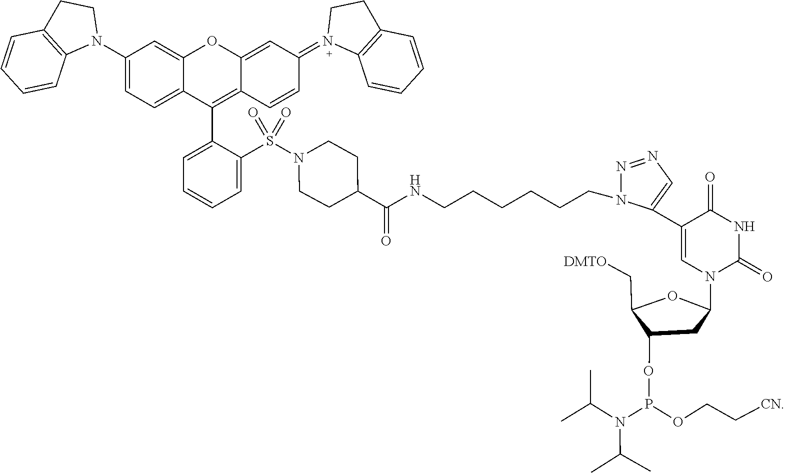

In embodiments, the compound is a compound as described herein, including in embodiments. In embodiments the compound is a compound described herein (e.g., in the examples section, figures, tables, or claims).

In an aspect is provided an oligonucleotide, or a salt thereof, having the formula:

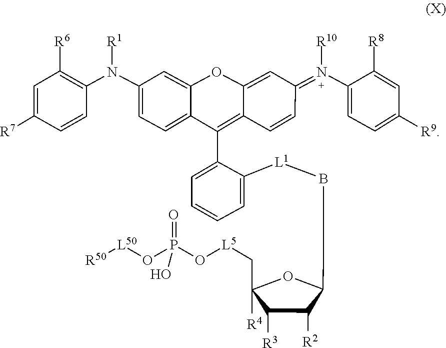

B, L¹, R¹, R², R⁴, R⁶, R⁷, R⁸, R⁹, and R¹⁰ are as described herein, including in embodiments.

L⁵ is a divalent oligonucleotide linker including from 4 to 40 nucleotides.

L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R⁵⁰ is a detectable moiety.

R^{30A} is —OR^{30A} or —O—P(NR^{3B}R^{3C})—OR^{3A}.

R^{30A} is hydrogen, a 3′ blocking moiety, or a monovalent oligonucleotide moiety.

R^{3A}, R^{3B}, and R^{3C} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the oligonucleotide has the formula:

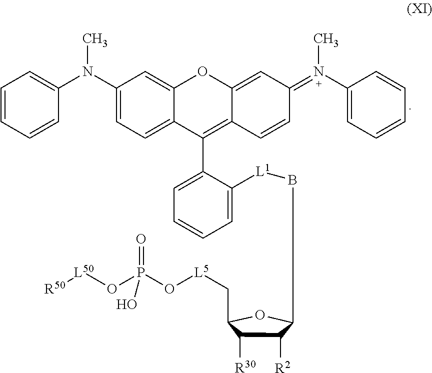

B, L¹, L⁵, L⁵⁰, R², R³⁰, and R⁵⁰ are as described herein, including in embodiments.

In embodiments, the oligonucleotide has the formula:

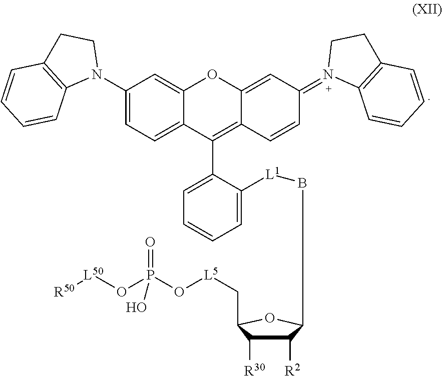

B, L¹, L⁵, L⁵⁰, R², R³⁰, and R⁵⁰ are as described herein, including in embodiments.

In embodiments, the oligonucleotide has the formula:

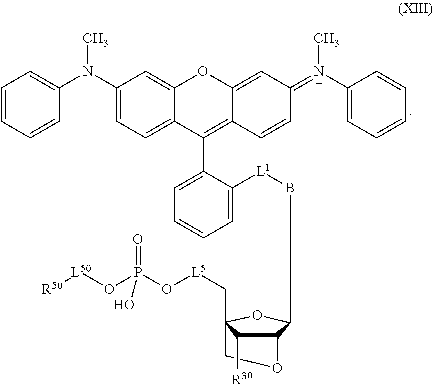

B, L¹, L⁵, L⁵⁰, R³⁰, and R⁵⁰ are as described herein, including in embodiments.

In embodiments, the oligonucleotide has the formula:

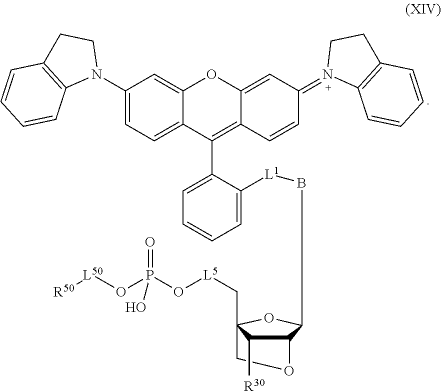

B, L¹, L⁵, L⁵⁰, R³⁰, and R⁵⁰ are as described herein, including in embodiments.

In embodiments, the oligonucleotide has the formula:

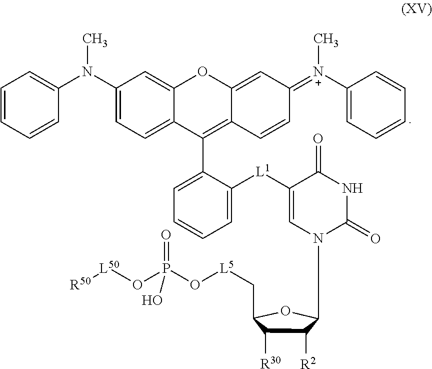

L¹, L⁵⁰, L R², R³⁰ and R⁵⁰ are as described herein, including in embodiments.

In embodiments, the oligonucleotide has the formula:

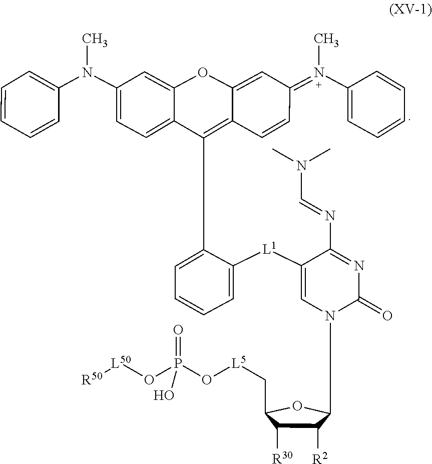

L¹, L⁵, L⁵⁰, R², R³⁰, and R⁵⁰ are as described herein, including in embodiments.

In embodiments, the oligonucleotide has the formula:

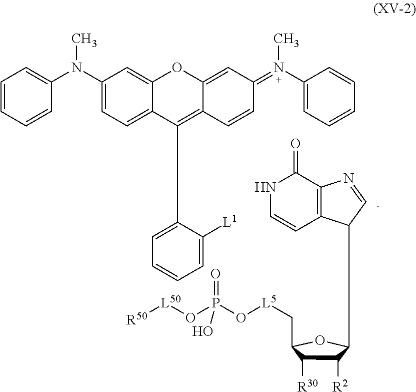

L¹, L⁵, L⁵⁰, R², R³⁰, and R⁵⁰ are as described herein, including in embodiments.

In embodiments, the oligonucleotide has the formula:

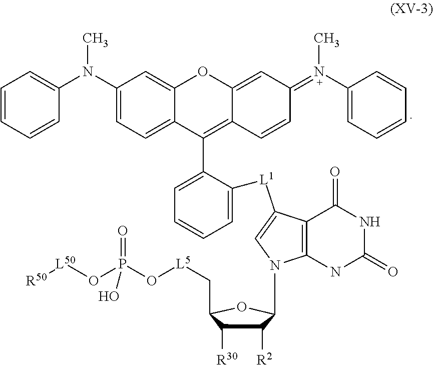

L¹, L⁵, L⁵⁰, R², R³⁰, and R⁵⁰ are as described herein, including in embodiments.

In embodiments, the oligonucleotide has the formula:

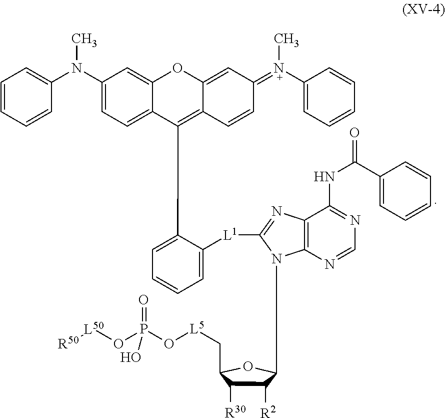

L¹, L⁵, L⁵⁰, R², R³⁰, and R⁵⁰ are as described herein, including in embodiments.

In embodiments, the oligonucleotide has the formula:

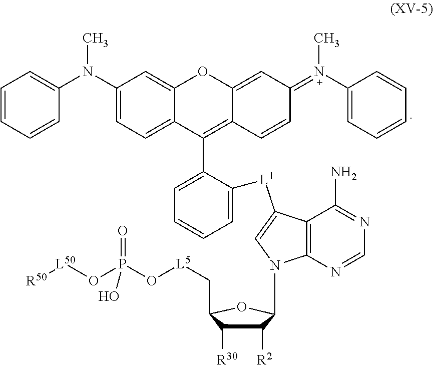

L¹, L⁵, L⁵⁰, R², R³⁰, and R⁵⁰ are as described herein, including in embodiments.

In embodiments, the oligonucleotide has the formula:

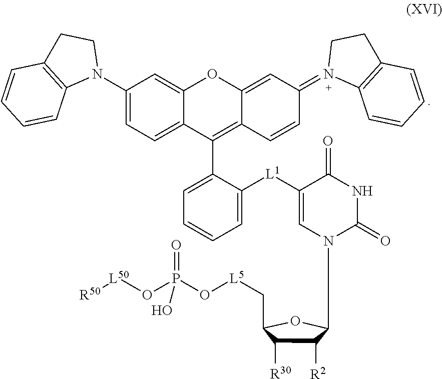

¹, L⁵, L⁵⁰, R², R³⁰, and R⁵⁰ are as described herein, including in embodiments.

In embodiments, the oligonucleotide has the formula:

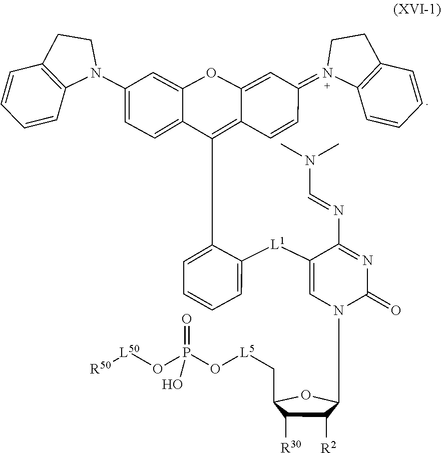

L¹, L⁵, L⁵⁰, R², R³⁰, and R⁵⁰ are as described herein, including in embodiments.

In embodiments, the oligonucleotide has the formula:

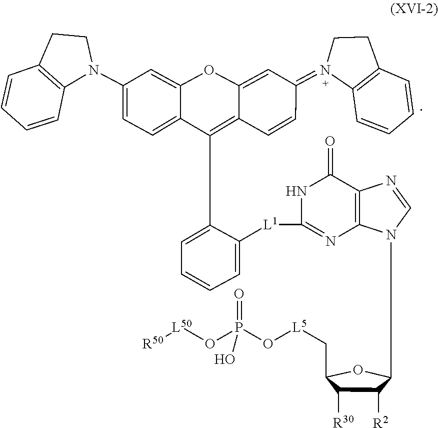

L¹, L⁵, L⁵⁰, R², R³⁰, and R⁵⁰ are as described herein, including in embodiments.

In embodiments, the oligonucleotide has the formula:

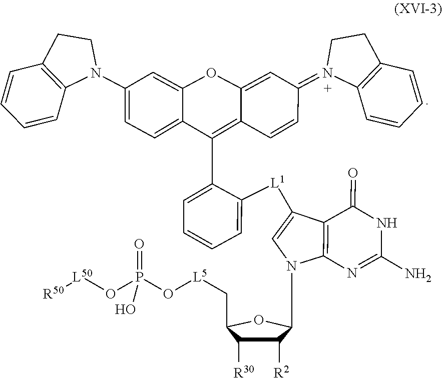

L¹, L⁵, L⁵⁰, R², R³⁰, and R⁵⁰ are as described herein, including in embodiments.

In embodiments, the oligonucleotide has the formula:

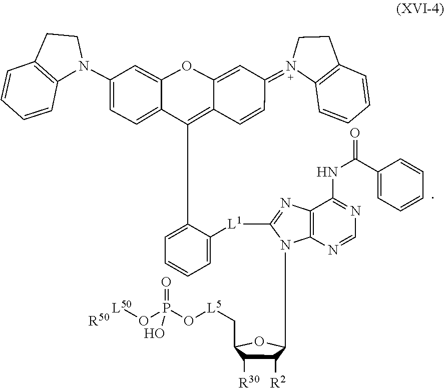

L¹, L⁵, L⁵⁰, R², R³⁰, and R⁵⁰ are as described herein, including in embodiments.

In embodiments, the oligonucleotide has the formula:

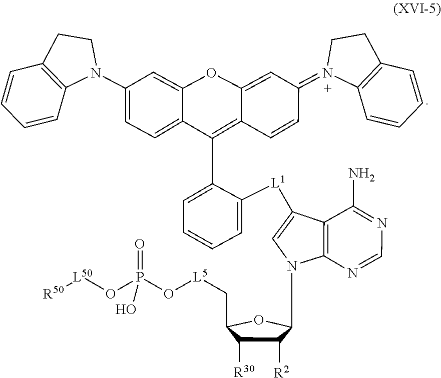

L¹, L⁵, L⁵⁰, R², R³⁰, and R⁵⁰ are as described herein, including in embodiments.

In embodiments, the oligonucleotide has the formula:

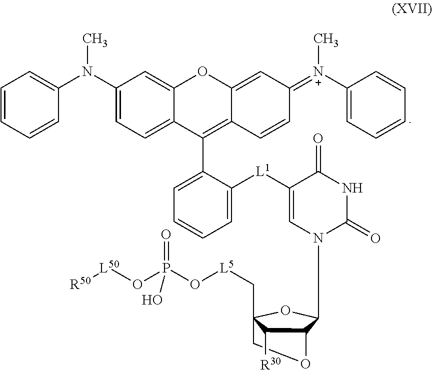

L¹, L⁵, L⁵⁰, R³⁰, and R⁵⁰ are as described herein, including in embodiments.

In embodiments, the oligonucleotide has the formula:

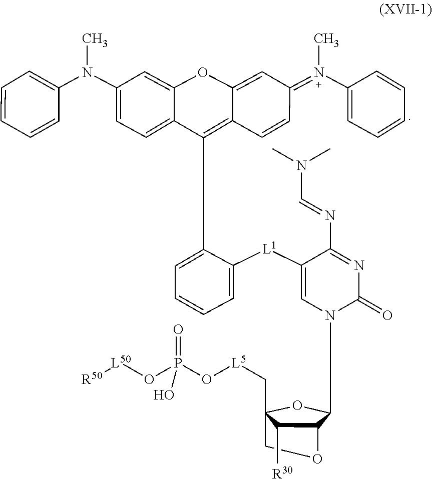

L¹, L⁵, L⁵⁰, R³⁰, and R⁵⁰ are as described herein, including in embodiments.

In embodiments, the oligonucleotide has the formula:

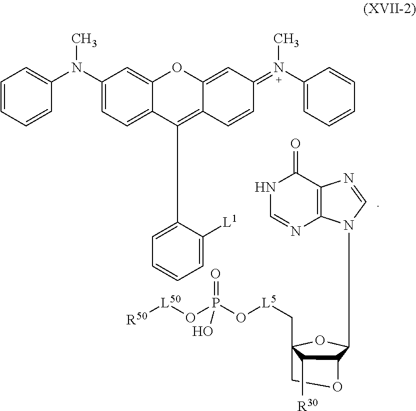

L¹, L⁵, L⁵⁰, R³⁰, and R⁵⁰ are as described herein, including in embodiments.

In embodiments, the oligonucleotide has the formula:

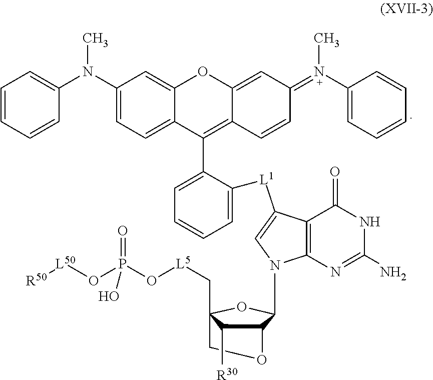

L¹, L⁵, L⁵⁰, R³⁰, and R⁵⁰ are as described herein, including in embodiments.

In embodiments, the oligonucleotide has the formula:

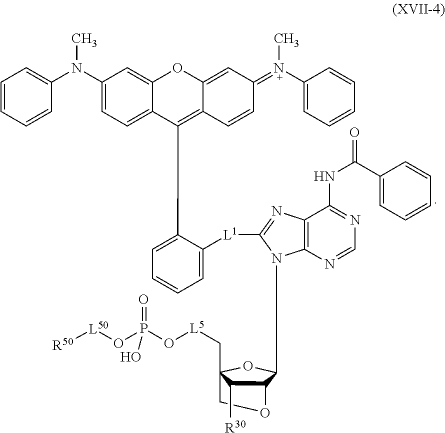

L¹, L⁵, L⁵⁰, R³⁰, and R⁵⁰ are as described herein, including in embodiments.

In embodiments, the oligonucleotide has the formula:

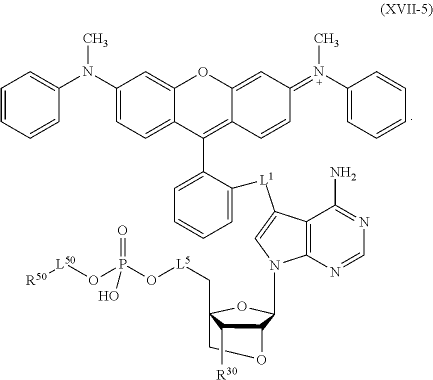

L¹, L⁵, L⁵⁰, R³⁰, and R⁵⁰ are as described herein, including in embodiments.

In embodiments, the oligonucleotide has the formula:

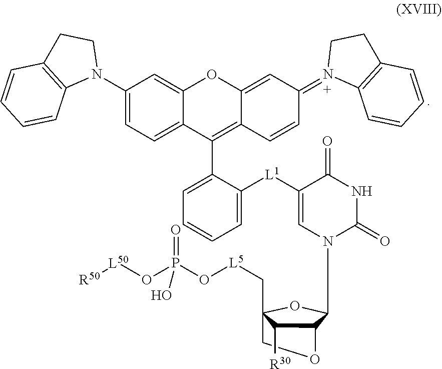

L¹, L⁵, L⁵⁰, R³⁰, and R⁵⁰ are as described herein, including in embodiments.

In embodiments, the oligonucleotide has the formula:

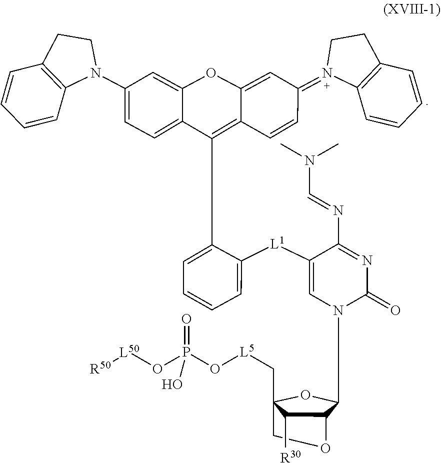

L¹, L⁵, L⁵⁰, R³⁰, and R⁵⁰ are as described herein, including in embodiments.

In embodiments, the oligonucleotide has the formula:

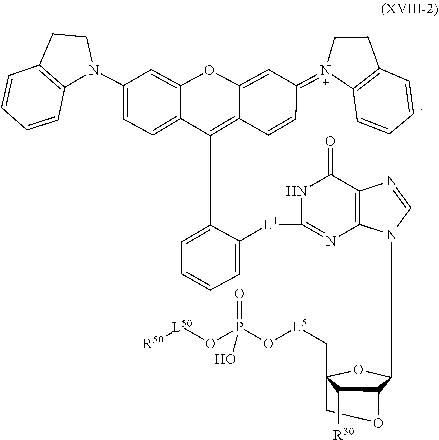

L¹, L⁵, L⁵⁰, R³⁰, and R⁵⁰ are as described herein, including in embodiments.

In embodiments, the oligonucleotide has the formula:

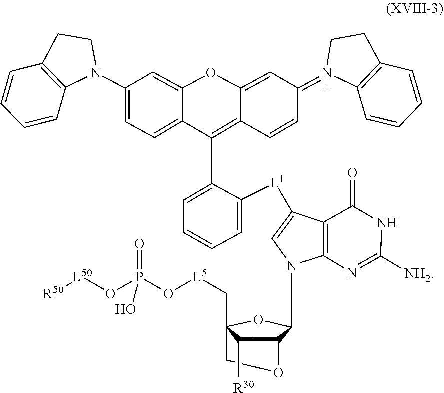

L¹, L⁵, L⁵⁰, R³⁰, and R⁵⁰ are as described herein, including in embodiments.

In embodiments, the oligonucleotide has the formula:

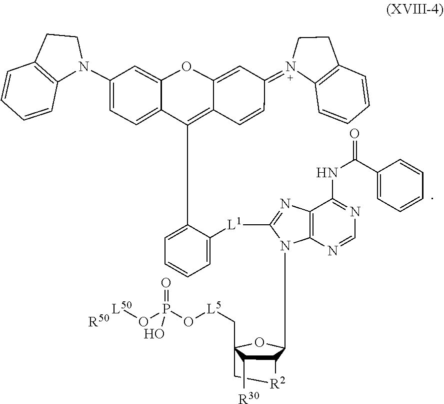

L¹, L⁵, L⁵⁰, R³⁰, and R⁵⁰ are as described herein, including in embodiments.

In embodiments, the oligonucleotide has the formula:

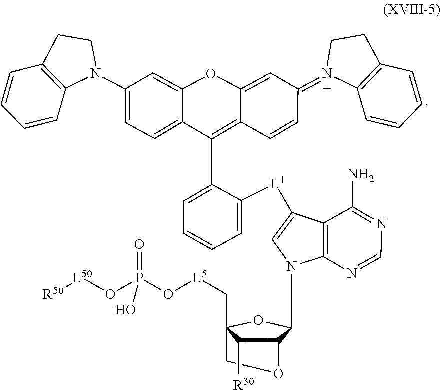

L¹, L⁵, L⁵⁰, R³⁰, and R⁵⁰ are as described herein, including in embodiments.

In embodiments, L⁵ includes from 11 to 30 nucleotides. In embodiments, L⁵ includes from 19 to 23 nucleotides. In embodiments, L⁵ includes from 4 to 14 nucleotides.

In embodiments, L⁵ includes from 6 to 12 nucleotides.

In embodiments, the nucleotides are DNA nucleotides. In embodiments, the nucleotides are RNA nucleotides.

In embodiments, a substituted L⁵⁰ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁵⁰ is substituted with a plurality of groups selected from substituent groups, size-limited substituent groups, and lower substituent groups; each substituent group, size-limited substituent group, and/or lower substituent group may optionally be different. In embodiments, when L⁵⁰ is substituted, it is substituted with at least one substituent group. In embodiments, when L⁵⁰ is substituted, it is substituted with at least one size-limited substituent group. In embodiments, when L⁵⁰ is substituted, it is substituted with at least one lower substituent group.

In embodiments, L⁵⁰ is a substituted 2 to 10 membered heteroalkylene. In embodiments, L⁵⁰ is an oxo-substituted 2 to 10 membered heteroalkylene. In embodiments, L⁵⁰ is

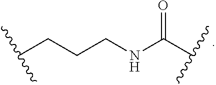 [SMILES: *CCCNC(*)=O]

In embodiments, R⁵⁰ is a fluorescent moiety (e.g., as described herein). In embodiments, R⁵⁰ is a monovalent form of FAM, a monovalent form of VIC, a monovalent form of ABY, a monovalent form of JUN, a monovalent form of AF647, a monovalent form of Cy5, a monovalent form of AF676, or a monovalent form of Cy5.5. In embodiments, R⁵⁰ is a monovalent form of Alexa Fluor 488. In embodiments, R⁵⁰ is a monovalent form of FAM. In embodiments, R⁵⁰ is a monovalent form of TET. In embodiments, R⁵⁰ is a monovalent form of HEX. In embodiments, R⁵⁰ is a monovalent form of JOE. In embodiments, R⁵⁰ is a monovalent form of VIC. In embodiments, R⁵⁰ is a monovalent form of Cy3. In embodiments, R⁵⁰ is a monovalent form of Alexa Fluor 555. In embodiments, R⁵⁰ is a monovalent form of NED. In embodiments, R⁵⁰ is a monovalent form of Alexa Fluor 546. In embodiments, R⁵⁰ is a monovalent form of TAMRA. In embodiments, R⁵⁰ is a monovalent form of Dye 3.0. In embodiments, R⁵⁰ is a monovalent form of ABY. In embodiments, R⁵⁰ is a monovalent form of PET. In embodiments, R⁵⁰ is a monovalent form of Dy3 3.5. In embodiments, R⁵⁰ is a monovalent form of Alexa Fluor 568. In embodiments, R⁵⁰ is a monovalent form of ROX. In embodiments, R⁵⁰ is a monovalent form of TX Red. In embodiments, R⁵⁰ is a monovalent form of Alexa Fluor 594. In embodiments, R⁵⁰ is a monovalent form of Dye 4.0. In embodiments, R⁵⁰ is a monovalent form of JUN. In embodiments, R⁵⁰ is a monovalent form of d-ROX. In embodiments, R⁵⁰ is a monovalent form of Alexa Fluor 647. In embodiments, R⁵⁰ is a monovalent form of Cy5. In embodiments, R⁵⁰ is a monovalent form of AF676. In embodiments, R⁵⁰ is a monovalent form of Cy5.5.

In embodiments, R³⁰ is —OR^{30A}. In embodiments, R³⁰ is —OH. In embodiments, R³⁰ is —O—P(NR^{3B}R^{3C})—OR^{3A}, wherein R^{3A}, R^{3B}, and R^{3C} are as described herein, including in embodiments. In embodiments, R³⁰ is

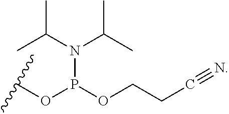 [SMILES: *OP(OCCC#N)N(C(C)C)C(C)C]

In embodiments, R^{30A} is hydrogen. In embodiments, R^{30A} is a 3′ blocking moiety. In embodiments, R^{30A} is a monovalent oligonucleotide moiety.

In embodiments, the 3′ blocking moiety is a monovalent form of dideoxycytidine (3′ddC). In embodiments, the 3′ blocking moiety is a monovalent form of dideoxyadenosine (ddA). In embodiments, the 3′ blocking moiety is 3′ Inverted dT. In embodiments, the 3′ blocking moiety is 3′ amino modifier. In embodiments, the 3′ blocking moiety is a monovalent form of QSY7. In embodiments, the 3′ blocking moiety is a monovalent form of QSY21. In embodiments, the 3′ blocking moiety is a monovalent form of QSY9. In embodiments, the 3′ blocking moiety is a monovalent form of BHQ1. In embodiments, the 3′ blocking moiety is a monovalent form of BHQ2. In embodiments, the 3′ blocking moiety is a monovalent form of BHQ3. In embodiments, the 3′ blocking moiety is a monovalent form of Dabcyl. In embodiments, the 3′ blocking moiety is a monovalent form of Dabsyl. In embodiments, the 3′ blocking moiety is a monovalent form of Eclipse. In embodiments, the 3′ blocking moiety is a monovalent form of BBQ-650. In embodiments, the 3′ blocking moiety is a monovalent form of Iowa Black RQ. In embodiments, the 3′ blocking moiety is a monovalent form of Iowa Black FQ. In embodiments, the 3′ blocking moiety is

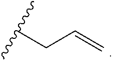 [SMILES: *CC=C]

In embodiments, the 3′ blocking moiety is

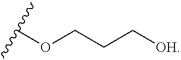 [SMILES: *OCCCO]

In embodiments, the 3′ blocking moiety is

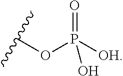 [SMILES: *OP(=O)(O)O]

In embodiments, the 3′ blocking moiety is

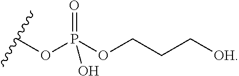 [SMILES: *OP(=O)(O)OCCCO]

In embodiments, the oligonucleotide has the formula:

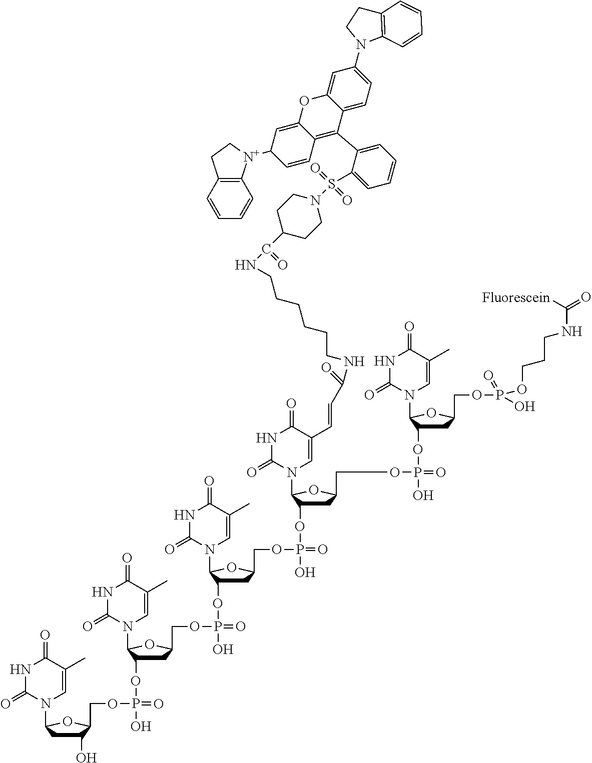

### III. Methods of Use

In an aspect is provided a method of forming a double-stranded nucleic acid, the method including contacting a target oligonucleotide with a nucleic acid probe including a quencher moiety (e.g., internal quencher moiety), thereby forming the double-stranded nucleic acid; wherein the nucleic acid probe has the formula:

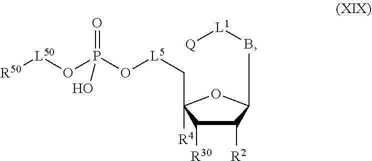

or a salt thereof. B, L¹, L⁵, L⁵⁰, R², R⁴, and R⁵⁰ are as described herein, including in embodiments. R³⁰ is —OR^{30A}, wherein R^{30A} is a monovalent oligonucleotide moiety. Q is a quencher moiety (e.g., as described herein). In embodiments, Q is an internal quencher moiety (e.g., as described herein).

In embodiments, the quencher moiety (e.g., internal quencher moiety) is a monovalent form of QSY7. In embodiments, the quencher moiety (e.g., internal quencher moiety) is a monovalent form of

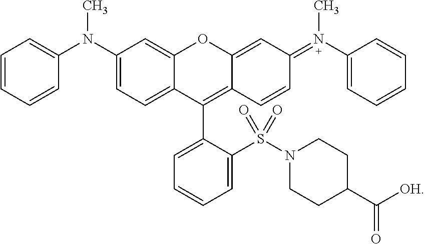

In embodiments, the quencher moiety (e.g., internal quencher moiety) is

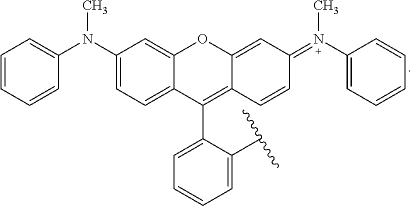

In embodiments, the quencher moiety (e.g., internal quencher moiety) is a monovalent form of QSY21. In embodiments, the quencher moiety (e.g., internal quencher moiety) is a monovalent form of

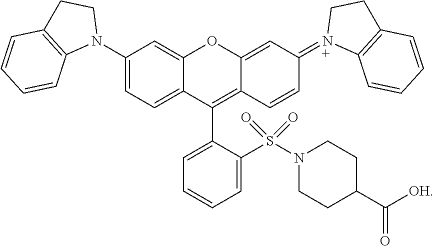

In embodiments, the quencher moiety (e.g., internal quencher moiety) is

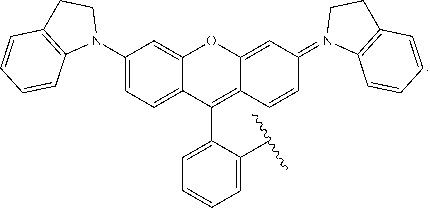

In embodiments, the quencher moiety (e.g., internal quencher moiety) is a monovalent form of QSY9. In embodiments, the quencher moiety (e.g., internal quencher moiety) is a monovalent form of

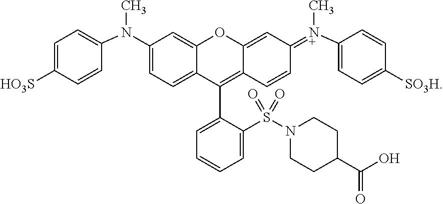

In embodiments, the quencher moiety (e.g., internal quencher moiety) is

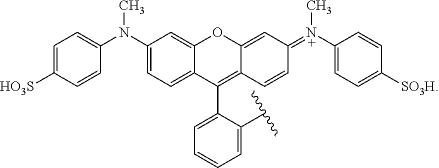

In embodiments, the quencher moiety (e.g., internal quencher moiety) is a monovalent form of BHQ1. In embodiments, the quencher moiety (e.g., internal quencher moiety) is a monovalent form of

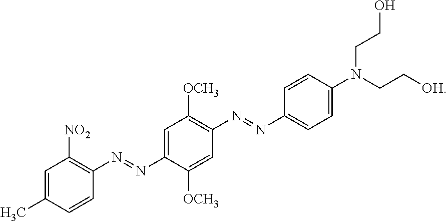 [SMILES: COc1cc(/N=N/c2ccc(C)cc2[N+](=O)[O-])c(OC)cc1/N=N/c1ccc(N(CCO)CCO)cc1]

In embodiments, the quencher moiety (e.g., internal quencher moiety) is

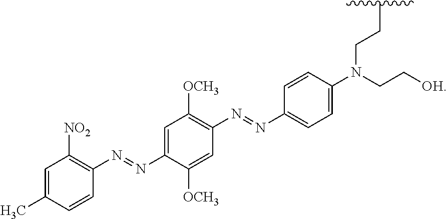 [SMILES: *CCN(CCO)c1ccc(/N=N/c2cc(OC)c(/N=N/c3ccc(C)cc3[N+](=O)[O-])cc2OC)cc1]

In embodiments, the quencher moiety (e.g., internal quencher moiety) is a monovalent form of BHQ2. In embodiments, the quencher moiety (e.g., internal quencher moiety) is a monovalent form of

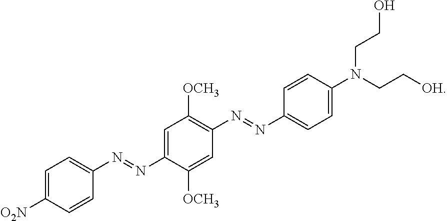 [SMILES: COc1cc(/N=N/c2ccc([N+](=O)[O-])cc2)c(OC)cc1/N=N/c1ccc(N(CCO)CCO)cc1]

In embodiments, the quencher moiety (e.g., internal quencher moiety) is

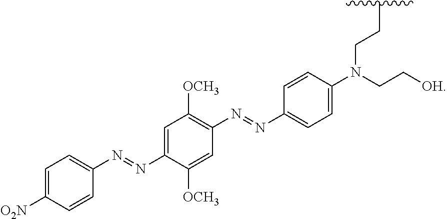 [SMILES: *CCN(CCO)c1ccc(/N=N/c2cc(OC)c(/N=N/c3ccc([N+](=O)[O-])cc3)cc2OC)cc1]

In embodiments, the quencher moiety (e.g., internal quencher moiety) is a monovalent form of BHQ3. In embodiments, the quencher moiety (e.g., internal quencher moiety) is a monovalent form of

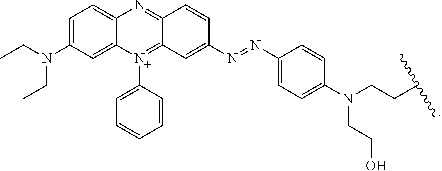

In embodiments, the quencher moiety (e.g., internal quencher moiety) is

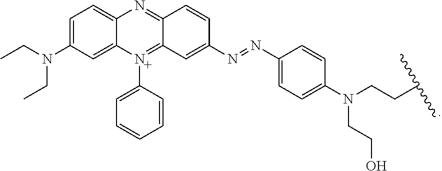

In embodiments, the quencher moiety (e.g., internal quencher moiety) is a monovalent form of Dabcyl. In embodiments, the quencher moiety (e.g., internal quencher moiety) is a monovalent form of

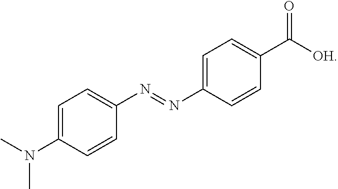 [SMILES: CN(C)c1ccc(/N=N/c2ccc(C(=O)O)cc2)cc1]

In embodiments, the quencher moiety (e.g., internal quencher moiety) is

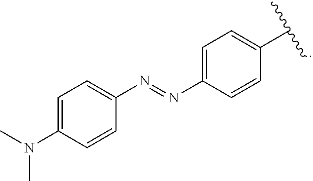 [SMILES: *c1ccc(/N=N/c2ccc(N(C)C)cc2)cc1]

In embodiments, the quencher moiety (e.g., internal quencher moiety) is a monovalent form of Dabsyl. In embodiments, the quencher moiety (e.g., internal quencher moiety) is a monovalent form of

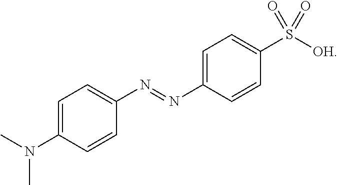 [SMILES: CN(C)c1ccc(/N=N/c2ccc(S(=O)(=O)O)cc2)cc1]

In embodiments, the quencher moiety (e.g., internal quencher moiety) is a monovalent form of Eclipse. In embodiments, the quencher moiety (e.g., internal quencher moiety) is a monovalent form of

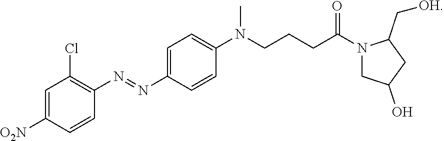 [SMILES: CN(CCCC(=O)N1CC(O)CC1CO)c1ccc(/N=N/c2ccc([N+](=O)[O-])cc2Cl)cc1]

In embodiments, the quencher moiety (e.g., internal quencher moiety) is

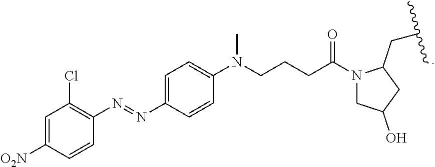 [SMILES: *CC1CC(O)CN1C(=O)CCCN(C)c1ccc(/N=N/c2ccc([N+](=O)[O-])cc2Cl)cc1]

In embodiments, the quencher moiety (e.g., internal quencher moiety) is a monovalent form of BBQ-650. In embodiments, the quencher moiety (e.g., internal quencher moiety) is

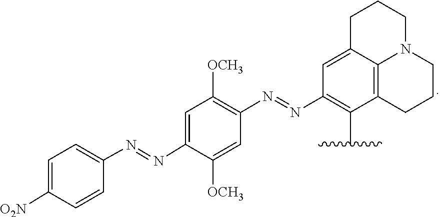 [SMILES: *c1c(/N=N/c2cc(OC)c(/N=N/c3ccc([N+](=O)[O-])cc3)cc2OC)cc2c3c1CCCN3CCC2]

In embodiments, the quencher moiety (e.g., internal quencher moiety) is a monovalent form of Iowa Black RQ. In embodiments, the quencher moiety (e.g., internal quencher moiety) is a monovalent form of Iowa Black FQ. In embodiments, the quencher moieties (e.g., internal quencher moieties) described in this paragraph may be used in any of the methods described herein.

In embodiments, Q is a quencher moiety (e.g., internal quencher moiety) having the formula:

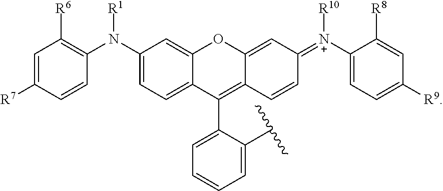

R¹, R⁶, R⁷, R⁸, R⁹, and R¹⁰ are as described herein, including in embodiments.

In embodiments, the method further includes contacting the double-stranded nucleic acid with a polymerase having exonuclease activity, thereby cleaving the quencher moiety (e.g., internal quencher moiety) from the detectable moiety. In embodiments, the method further includes detecting a signal generated by the detectable moiety. In embodiments, the detectable moiety is a fluorescent moiety (e.g., as described herein). In embodiments, the method further includes amplifying the target oligonucleotide using PCR. In embodiments, the PCR is quantitative PCR. In embodiments, the PCR is digital PCR.

In an aspect is provided a method of detecting nucleic acids in a sample, including: (i) providing a reaction mixture, the reaction mixture including: at least a portion of the sample, a first probe detectably labeled with a first label configured to generate a first fluorescence signal, a second probe detectably labeled with a second label configured to generate a second fluorescence signal, wherein the first and second probes have different sequences, wherein the first and second labels are identical and/or generate substantially identical fluorescence; and wherein the second probe has the formula:

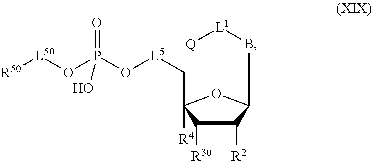

or a salt thereof, wherein Q, B, L¹, L⁵, L⁵⁰, R², and R⁴ are as described herein, including in embodiments; R³⁰ is —OR^{30A}, wherein R^{30A} is a monovalent oligonucleotide moiety; R⁵⁰ is the second label; (ii) subjecting the reaction mixture to an amplification process comprising a first set of reaction conditions and a second set of reaction conditions, the first set of reaction conditions being different than the second set of reaction conditions; and (iii) determining the presence of the first and/or second nucleic acid targets in the sample by measuring a fluorescence signal during the first set of reaction conditions, the fluorescence signal during the first set of reaction conditions being correlated with specific interaction or lack of interaction between the first probe and a first nucleic acid target, measuring a fluorescence signal during the second set of reaction conditions, the fluorescence signal during the second set of reaction conditions being correlated with specific interaction or lack of interaction between the first probe and a first nucleic acid target and with specific or lack of interaction between the second probe and a second nucleic acid target, and estimating the presence and/or amount of the first nucleic acid target and second nucleic acid target.

In an aspect is provided a method of detecting nucleic acids in a sample, including: (i) providing a reaction mixture, the reaction mixture including: at least a portion of the sample, a first probe detectably labeled with a first label configured to generate a first fluorescence signal that is indicative of the presence or absence of a first nucleic acid target, a second probe detectably labeled with a second label configured to generate a second fluorescence signal that is indicative of the presence or absence of a second nucleic acid target, wherein the first and second probes have different sequences, wherein the first and second labels are identical and/or generate substantially identical fluorescence; and wherein the second probe has the formula:

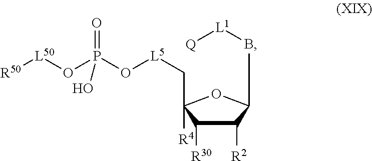

or a salt thereof, wherein Q, B, L, L⁵, L⁵⁰, R², and R⁴ are as described herein, including in embodiments; R³⁰ is —OR^{30A}, wherein R^{30A} is a monovalent oligonucleotide moiety; R⁵⁰ is the second label; (ii) subjecting the reaction mixture to an amplification process including a first set of reaction conditions and a second set of reaction conditions, the first set of reaction conditions being different than the second set of reaction conditions; and (iii) determining any presence and/or amount of the first and/or second nucleic acid targets in the reaction mixture by measuring during the first set of reaction conditions a first total fluorescence signal that includes any first fluorescence signal if present and any second fluorescence signal if present, measuring during the second set of reaction conditions a second total fluorescence signal including any first fluorescence signal if present and any second fluorescence signal if present, and estimating the first fluorescence signal and/or second fluorescence signal based on the first and second total fluorescence signals.

In embodiments, both the first and second probes are subjected to excitation at the same wavelength and/or both the first and second probes are subjected to excitation during detection of their respective first and second fluorescence signals.

In embodiments, measuring the fluorescence signal during the first set of reaction conditions includes measuring a combined signal including the first and second fluorescence signals during the first set of reaction conditions to obtain a first total fluorescence signal; measuring the fluorescence signal during the second set of reaction conditions includes measuring a combined signal comprising the first and second fluorescence signals during the second set of reaction conditions to obtain a second total fluorescence signal; and estimating the presence and/or amount of the first nucleic acid target and the second nucleic acid target includes estimating the first fluorescence signal and/or second fluorescence signal based on the first and second total fluorescence signals.

In embodiments, the second fluorescence signal differs between the first and second set of reaction conditions to a greater degree than the first fluorescence signal differs between the first and second set of reaction conditions.

In embodiments, the first total fluorescence value includes (i) fluorescence from first label that is free and unquenched within the reaction mixture and emitted as a result of the first label being cleaved following hybridization of the first probe to the first amplicon, and (ii) background fluorescence of the second label, and the second total fluorescence value is based on (i) fluorescence from first label that is free and unquenched within the reaction mixture and emitted as a result of the first label being cleaved following hybridization of the first probe to the first amplicon, and (ii) fluorescence from the second label, above the background fluorescence of the second label, emitted as a result of hybridization of the second probe to the second amplicon.

In embodiments, the method further includes calculating an amount of the first nucleic acid target based on the first fluorescent signal; and calculating an amount of the second nucleic acid target based on the second fluorescent signal.

In embodiments, when the first nucleic acid target is present in the reaction mixture, the first fluorescent signal is above a background level during both the first and second sets of reaction conditions. In embodiments, when the second nucleic acid target is present in the reaction mixture, the second fluorescent signal is above a background level during the second set of reaction conditions but not during the first set of reaction conditions.

In embodiments, the first set of reaction conditions includes a first measurement temperature at which the first fluorescence signal is measured, and the second set of reaction conditions includes a second, different measurement temperature at which the second fluorescence signal is measured. In embodiments, the first and second measurement temperatures differ by at least about 10° C. or more. In embodiments, the first and second measurement temperatures differ by at least about 15° C. or more. In embodiments, the first and second measurement temperatures differ by at least about 20° C. or more. In embodiments, the first and second measurement temperatures differ by at least about 25° C. or more. In embodiments, the first and second measurement temperatures differ by at least about 30° C. or more. In embodiments, at least one of the first or second measurement temperatures is a denaturation temperature at which DNA in the reaction mixture is denatured. In embodiments, the denaturation temperature is about 90° C. or above.

In embodiments, the reaction mixture is subjected to multiple amplification cycles each including the first and second set of reaction conditions. In embodiments, the amplification process includes thermal cycling.

In embodiments, the first set of reaction conditions includes a denaturation step at a first temperature of the thermal cycling. In embodiments, the second set of reaction conditions includes an annealing and/or extension step (“annealing/extension step”) at a second temperature of the thermal cycling, the second temperature being lower than the first temperature.

In embodiments, the first probe is a cleavable probe. In embodiments, the first fluorescent signal increases when the cleavable probe is cleaved during an annealing/extension step. In embodiments, the first probe includes a fluorophore (e.g., as described herein) and a quencher (e.g., as described herein), and wherein the first probe is configured such that fluorescence from the fluorophore is quenched by the quencher until the probe is cleaved during an annealing/extension step of the amplification process. In embodiments, the first probe is a TaqMan probe.

In embodiments, the second probe is a non-cleavable probe. In embodiments, L⁵ forms a stem-loop structure when the second probe is single-stranded. In embodiments, Q and R⁵⁰ are spaced apart from one another such that R⁵⁰ is quenched when the second probe is single stranded and unquenched when the second probe is incorporated into a double stranded amplicon. In embodiments, both Q and R⁵⁰ are disposed at or near the stem loop portion of the second probe.

In embodiments, the reaction mixture further includes: a first primer pair complementary to a first nucleic acid target of the nucleic acids or its complement, the first nucleic acid target being configured to generate a first amplicon with which the first probe can hybridize; and a second primer pair complementary to a second nucleic acid target of the nucleic acids or its complement, the second nucleic acid target being configured to generate a second amplicon with which the second probe can hybridize. In embodiments, the second primer pair includes a primer with a tail. In embodiments, the tail forms the 5′ end of the primer with the tail. In embodiments, the second probe can hybridize to the tail or to its complement.

In embodiments, the amplification process utilizes a series of thermal cycling steps that includes at least three different target temperatures. In embodiments, the amplification process includes a denaturation temperature and multiple different annealing/extension temperatures that vary throughout the amplification process. In embodiments, a first series of denaturation and annealing/extension steps are carried out at a first annealing/extension temperature, and wherein a second series of denaturation and annealing/extension steps are carried out at a second, different annealing/extension temperature. In embodiments, the first annealing/extension temperature is higher than the second annealing/extension temperature. In embodiments, the first series of denaturation and annealing/extension steps are cycled a greater number of times than the second series of denaturation and annealing/extension steps.

In embodiments, the amplification process further includes a third series of denaturation and annealing/extension steps carried out using a third annealing/extension temperature. In embodiments, the third annealing/extension temperature is the same as the first annealing/extension temperature. In embodiments, the third series of denaturation and annealing/extension steps are cycled a greater number of times than the first series of denaturation and annealing/extension steps. In embodiments, the denaturation temperature is the same for each series of denaturation and annealing/extension steps.

In embodiments, the second primer pair includes the tailed primer and a non-tailed primer, and wherein the tailed primer and non-tailed primer are provided at different concentrations. In embodiments, the non-tailed primer is provided at a greater concentration than the tailed primer. In embodiments, the non-tailed primer is provided at a concentration that is about 2× to about 30× the concentration of the tailed primer. In embodiments, the non-tailed primer is provided at a concentration that is about 5× to about 25× the concentration of the tailed primer. In embodiments, the non-tailed primer is provided at a concentration that is about 10× to about 20× the concentration of the tailed primer. In embodiments, the second probe is provided at a concentration that is different from the concentration of the tailed primer and the concentration of the non-tailed primer. In embodiments, the second probe is provided at a concentration that is greater than the concentration of the tailed primer. In embodiments, the second probe is provided at a concentration that is less than the concentration of the non-tailed primer. In embodiments, the second probe is provided at a concentration that is about 2× to about 10× the concentration of the tailed primer. In embodiments, the second probe is provided at a concentration that is about 3× to about 7.5× the concentration of the tailed primer.

In embodiments, a melting temperature (Tₘ) of the first probe and a Tₘ of the second probe are within about 8° C. of each other. In embodiments, a melting temperature (Tₘ) of the first probe and a Tₘ of the second probe are within about 6° C. of each other. In embodiments, a melting temperature (Tₘ) of the first probe and a Tₘ of the second probe are within about 4° C. of each other. In embodiments, a melting temperature (Tₘ) of the first probe and a Tₘ of the second probe are within about 2° C. of each other.

In embodiments, the amplification process cycles between two target temperatures for multiple cycles of the amplification process. In embodiments, the amplification process cycles between two target temperatures for at least 5% of the cycles of the amplification process. In embodiments, the amplification process cycles between two target temperatures for at least 10% of the cycles of the amplification process. In embodiments, the amplification process cycles between two target temperatures for at least 15% of the cycles of the amplification process. In embodiments, the amplification process cycles between two target temperatures for at least 20% of the cycles of the amplification process. In embodiments, the amplification process cycles between two target temperatures for at least 25% of the cycles of the amplification process. In embodiments, the amplification process cycles between two target temperatures for at least 30% of the cycles of the amplification process. In embodiments, the amplification process cycles between two target temperatures for at least 35% of the cycles of the amplification process. In embodiments, the amplification process cycles between two target temperatures for at least 40% of the cycles of the amplification process. In embodiments, the amplification process cycles between two target temperatures for at least 45% of the cycles of the amplification process. In embodiments, the amplification process cycles between two target temperatures for at least 50% of the cycles of the amplification process. In embodiments, the amplification process cycles between two target temperatures for at least 55% of the cycles of the amplification process. In embodiments, the amplification process cycles between two target temperatures for at least 60% of the cycles of the amplification process. In embodiments, the amplification process cycles between two target temperatures for at least 65% of the cycles of the amplification process. In embodiments, the amplification process cycles between two target temperatures for at least 70% of the cycles of the amplification process. In embodiments, the amplification process cycles between two target temperatures for at least 75% of the cycles of the amplification process. In embodiments, the amplification process cycles between two target temperatures for at least 80% of the cycles of the amplification process. In embodiments, the amplification process cycles between two target temperatures for at least 85% of the cycles of the amplification process. In embodiments, the amplification process cycles between two target temperatures for at least 90% of the cycles of the amplification process. In embodiments, the amplification process cycles between two target temperatures for at least 95% of the cycles of the amplification process.

In embodiments, the reaction mixture is added to or formed within a plurality of reaction volumes, and wherein the amplification process is a digital PCR (dPCR) process.

In embodiments, measuring the fluorescence signal during the first set of reaction conditions includes a first end-point measurement at the first set of reaction conditions, and wherein measuring the fluorescence signal during the second set of reaction conditions includes a second end-point measurement at the second set of reaction conditions.

In embodiments, estimating the presence and/or amount of the first nucleic acid target and the second nucleic acid target includes: categorizing the plurality of reaction volumes according to measured signal at the first end-point measurement and according to measured signal at the second end-point measurement; and based on the categorizations, determining a count for reaction volumes in which the first probe showed activity and a count for reaction volumes in which the second probe showed activity.

In an aspect is provided a method of detecting nucleic acids in a sample, including: providing a reaction mixture, the reaction mixture including a primer pair complementary to a nucleic acid target or its complement for generating an amplicon, and a non-cleavable probe configured to hybridize to the amplicon, the probe including a detectable label configured to provide a fluorescent signal that corresponds to an amount of generated amplicon, subjecting the reaction mixture to an amplification process to generate the amplicons, wherein the label generates fluorescence without cleavage of the probe during the amplification process, and wherein the amplification process utilizes a series of thermal cycling steps that includes at least three different target temperatures; and measuring the fluorescent signal from the probe; wherein the probe has the formula:

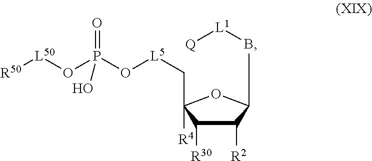

or a salt thereof, wherein Q, B, L, L⁵, L⁵⁰, R², and R⁴ are as described herein, including in embodiments; R³⁰ is —OR^{30A}, wherein R^{30A} is a monovalent oligonucleotide moiety; and R⁵⁰ is the detectable label.

In embodiments, the method further includes quantitating an amount of the nucleic acid target based on the measured fluorescent signal.

In embodiments, the probe includes a stem-loop portion capable of forming a stem-loop structure when the probe is single stranded. In embodiments, Q and R⁵⁰ are spaced such that R⁵⁰ is quenched when the probe is single stranded but enabled when the probe is incorporated into a double stranded amplicon. In embodiments, both Q and R⁵⁰ are at or near the stem loop portion of the probe.

In embodiments, the primer pair includes a primer with a tail. In embodiments, the tail forms the 5′ end of the primer with the tail. In embodiments, the probe is configured to hybridize to the tail or to its complement. In embodiments, a 3′ portion of the probe is configured to hybridize to the tail or its complement.

In embodiments, the amplification process includes a denaturation temperature and multiple different annealing/extension temperatures that vary throughout the amplification process. In embodiments, a first series of denaturation and annealing/extension steps are carried out at a first annealing/extension temperature, and wherein a second series of denaturation and annealing/extension steps are carried out at a second, different annealing/extension temperature. In embodiments, the first annealing/extension temperature is higher than the second annealing/extension temperature. In embodiments, the first series of denaturation and annealing/extension steps are cycled a greater number of times than the second series of denaturation and annealing/extension steps.

In embodiments, the amplification process further includes a third series of denaturation and annealing/extension steps carried out using a third annealing/extension temperature. In embodiments, the third annealing/extension temperature is the same as the first annealing/extension temperature. In embodiments, the third series of denaturation and annealing/extension steps are cycled a greater number of times than the first series of denaturation and annealing/extension steps. In embodiments, the denaturation temperature is the same for each series of denaturation and annealing/extension steps.

In embodiments, the primer pair includes the tailed primer and a non-tailed primer, and wherein the tailed primer and non-tailed primer are provided at different concentrations. In embodiments, the non-tailed primer is provided at a greater concentration than the tailed primer. In embodiments, the non-tailed primer is provided at a concentration that is about 2× to about 30× the concentration of the tailed primer. In embodiments, the non-tailed primer is provided at a concentration that is about 5× to about 25× the concentration of the tailed primer. In embodiments, the non-tailed primer is provided at a concentration that is about 10× to about 20× the concentration of the tailed primer. In embodiments, the probe is provided at a concentration that is different from the concentration of the tailed primer and the concentration of the non-tailed primer. In embodiments, the probe is provided at a concentration that is greater than the concentration of the tailed primer. In embodiments, the probe is provided at a concentration that is less than the concentration of the non-tailed primer. In embodiments, the probe is provided at a concentration that is about 2× to about 10× the concentration of the non-tailed primer. In embodiments, the probe is provided at a concentration that is about 3× to about 7.5× the concentration of the non-tailed primer.

In an aspect is provided a method of detecting nucleic acids in a sample, including: providing a reaction mixture, the reaction mixture including a primer pair targeted to a nucleic acid target for generating an amplicon, the primer pair including a tailed primer and a non-tailed primer provided at different concentrations, and a non-cleavable probe configured to hybridize to the amplicon, the probe including a detectable label configured to generate a fluorescent signal that corresponds to an amount of generated amplicon, subjecting the reaction mixture to an amplification process to generate the amplicons, wherein the probe generates fluorescence without being cleaved during the amplification process; and measuring the fluorescent signal from the probe; wherein the probe has the formula:

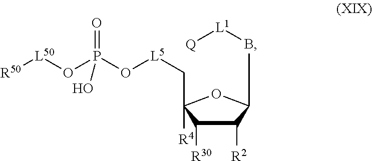

or a salt thereof, wherein Q, B, L¹, L⁵, L⁵⁰, R², and R⁴ are as described herein, including in embodiments; R³⁰ is —OR^{30A}, wherein R^{30A} is a monovalent oligonucleotide moiety; and R⁵⁰ is the detectable label.

In embodiments, the non-tailed primer is provided at a greater concentration than the tailed primer. In embodiments, the non-tailed primer is provided at a concentration that is about 2× to about 30× the concentration of the tailed primer. In embodiments, the non-tailed primer is provided at a concentration that is about 5× to about 25× the concentration of the tailed primer. In embodiments, the non-tailed primer is provided at a concentration that is about 10× to about 20× the concentration of the tailed primer. In embodiments, the probe is provided at a concentration that is different from the concentration of the tailed primer and the concentration of the non-tailed primer. In embodiments, the probe is provided at a concentration that is greater than the concentration of the tailed primer. In embodiments, the probe is provided at a concentration that is less than the concentration of the non-tailed primer. In embodiments, the probe is provided at a concentration that is about 2× to about 10× the concentration of the non-tailed primer. In embodiments, the probe is provided at a concentration that is about 3× to about 7.5× the concentration of the non-tailed primer.

In an aspect is provided a method of detecting the presence or amount of a first and/or second target in a reaction mixture, including: including a first probe and a second probe in the reaction mixture, wherein the first probe can specifically interact with a first target and includes a first label that can produce a first detectable signal, and the second probe can specifically interact with a second target and includes a second label that can produce a second detectable signal; wherein the second probe has the formula:

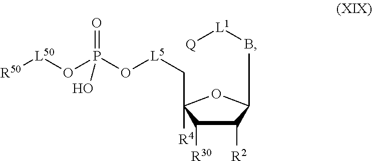

or a salt thereof, wherein Q, B, L, L⁵, L⁵⁰, R², and R⁴ are as described herein, including in embodiments; R³⁰ is —OR^{30A}, wherein R^{30A} is a monovalent oligonucleotide moiety; and R⁵⁰ is the second label; allowing specific interaction of the first and second probe with any first and second target, respectively, in the reaction mixture; measuring a first total signal through an optical filter under a first set of conditions, wherein the first total signal includes the first and second detectable signals from the first and second labels, and wherein under the first set of conditions, the first detectable signal is increased as a result of specific interaction of the first probe with the first target, but the second detectable signal is not increased as a result of specific interaction of the second probe with the second target; measuring a second total signal through the same optical filter under a second set of conditions, wherein the second total signal includes the first and second detectable signals from the first and second labels, and wherein under the second set of conditions, the second detectable signal is increased as a result of specific interaction of the second probe with the second target; and assessing the presence or amount of the first and/or second target, by estimating the first detectable signal and the second detectable signal based on both the first total signal and the second total signal.

In embodiments, the first and second labels are identical and/or generate substantially identical fluorescence. In embodiments, the second fluorescence signal differs between the first and second set of conditions to a greater degree than the first fluorescence signal differs between the first and second set of conditions.

In embodiments, the first probe is a cleavable probe. In embodiments, the first detectable signal increases when the cleavable probe is cleaved. In embodiments, the first probe includes a fluorophore (e.g., as described herein) and a quencher (e.g., as described herein), and wherein the first probe is configured such that fluorescence from the fluorophore is quenched by the quencher until the probe is cleaved. In embodiments, the first probe is a TaqMan probe.

In embodiments, the second probe is a non-cleavable probe. In embodiments, L⁵ forms a stem-loop structure when the second probe is single-stranded. In embodiments, R⁵⁰ is a fluorophore, and Q and R⁵⁰ are spaced apart from one another such that R⁵⁰ is quenched when the second probe is single stranded and unquenched when the second probe is incorporated into a double stranded amplicon. In embodiments, both Q and R⁵⁰ are disposed at or near the stem loop portion of the second probe.

In embodiments, a melting temperature (Tₘ) of the first probe and a Tₘ of the second probe are within about 8° C. of each other. In embodiments, a melting temperature (Tₘ) of the first probe and a Tₘ of the second probe are within about 6° C. of each other. In embodiments, a melting temperature (Tₘ) of the first probe and a Tₘ of the second probe are within about 4° C. of each other. In embodiments, a melting temperature (Tₘ) of the first probe and a Tₘ of the second probe are within about 2° C. of each other.

In embodiments, the first set of conditions includes a first measurement temperature at which the first fluorescence signal is measured, and the second set of conditions includes a second, different measurement temperature at which the second fluorescence signal is measured. In embodiments, the first and second measurement temperatures differ by at least about 10° C. or more. In embodiments, the first and second measurement temperatures differ by at least about 15° C. or more. In embodiments, the first and second measurement temperatures differ by at least about 20° C. or more. In embodiments, the first and second measurement temperatures differ by at least about 25° C. or more. In embodiments, the first and second measurement temperatures differ by at least about 30° C. or more. In embodiments, at least one of the first or second measurement temperatures is a denaturation temperature at which DNA in the reaction mixture is denatured. In embodiments, the denaturation temperature is about 90° C. or above.

In embodiments, the method further includes thermal cycling of the reaction mixture between two target temperatures for multiple cycles. In embodiments, the thermal cycling cycles between two target temperatures for at least 5% of the cycles. In embodiments, the thermal cycling cycles between two target temperatures for at least 10% of the cycles. In embodiments, the thermal cycling cycles between two target temperatures for at least 15% of the cycles. In embodiments, the thermal cycling cycles between two target temperatures for at least 20% of the cycles. In embodiments, the thermal cycling cycles between two target temperatures for at least 25% of the cycles. In embodiments, the thermal cycling cycles between two target temperatures for at least 30% of the cycles. In embodiments, the thermal cycling cycles between two target temperatures for at least 35% of the cycles. In embodiments, the thermal cycling cycles between two target temperatures for at least 40% of the cycles. In embodiments, the thermal cycling cycles between two target temperatures for at least 45% of the cycles. In embodiments, the thermal cycling cycles between two target temperatures for at least 50% of the cycles. In embodiments, the thermal cycling cycles between two target temperatures for at least 55% of the cycles. In embodiments, the thermal cycling cycles between two target temperatures for at least 60% of the cycles. In embodiments, the thermal cycling cycles between two target temperatures for at least 65% of the cycles. In embodiments, the thermal cycling cycles between two target temperatures for at least 70% of the cycles. In embodiments, the thermal cycling cycles between two target temperatures for at least 75% of the cycles. In embodiments, the thermal cycling cycles between two target temperatures for at least 80% of the cycles. In embodiments, the thermal cycling cycles between two target temperatures for at least 85% of the cycles. In embodiments, the thermal cycling cycles between two target temperatures for at least 90% of the cycles. In embodiments, the thermal cycling cycles between two target temperatures for at least 95% of the cycles.

### IV. Intra-Channel Multiplexing (Multiplexing Utilizing Probes with Labels Having Spectral Similarity)

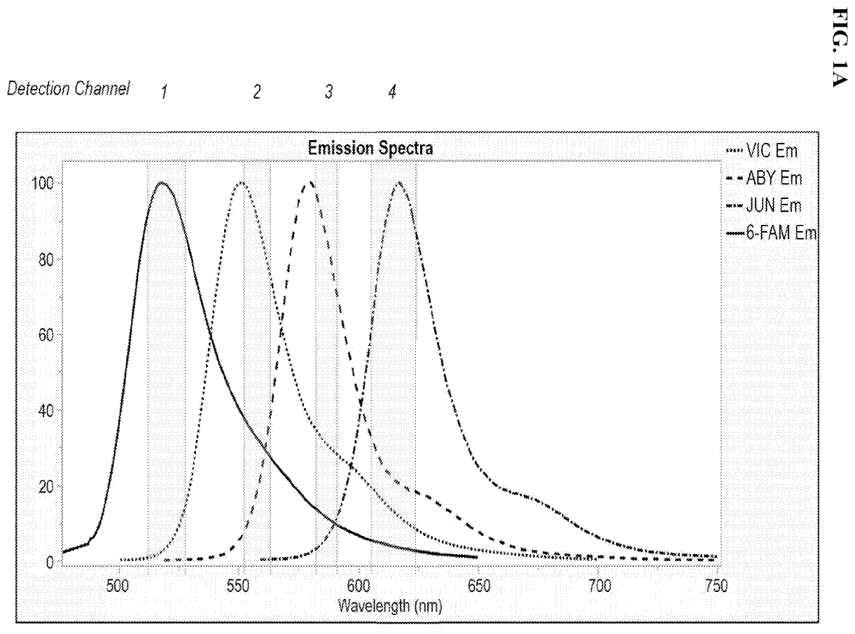

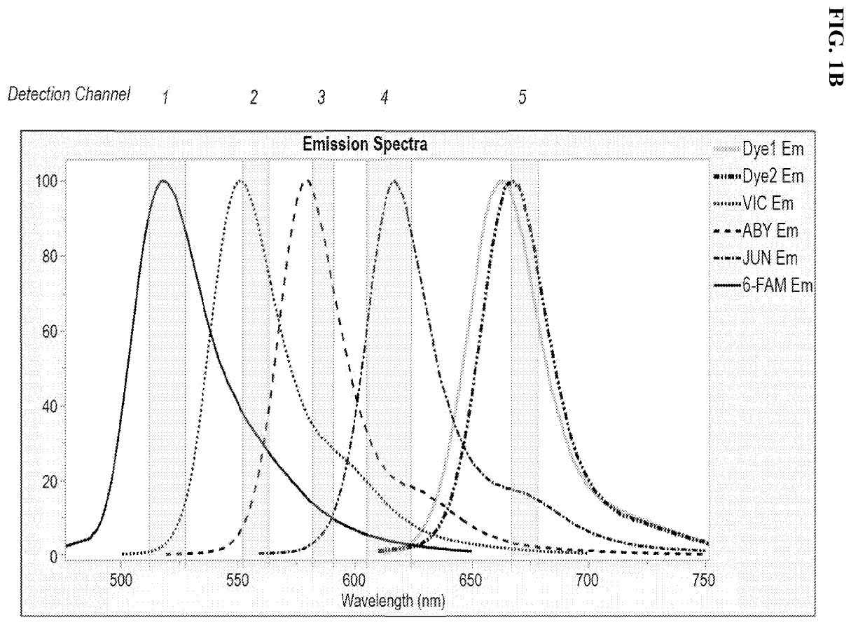

FIG. 1A illustrates emission spectra for various fluorescent dyes commonly used in nucleic acid detection assays. As discussed herein, conventional multiplex assays assign each dye to a separate target, and then determine the presence and/or amount of each target by measuring the fluorescence signal in separate detection channels each corresponding to the emission wavelength of the corresponding dye. As shown, there is typically a substantial amount of overlap in the emission spectra of the dyes. While multiplexed dyes are typically selected with the intent to minimize spectral overlap, the finite nature of the emission spectrum places practical limits on the number of separate dyes that can be combined in the same multiplex assay, and therefore limits the number of different targets that can be detected and/or measured.

Embodiments described herein solve one or more of the foregoing problems by providing multiple detectable signals, each associated with a different assay target or set of targets, within the same detection channel. The multiple detectable signals can be separately resolved and independently analyzed to thereby allow detection and/or quantification of each target. By allowing multiple targets to be assayed within the same detection channel, the disclosed embodiments can beneficially increase the plexy of multiplex assays without relying on additional dyes and concomitant issues of increased spectral overlap. Similarly, embodiments described herein can beneficially decrease the number of separate dyes required in a multiplex assay without lowering the plexy of the assay.

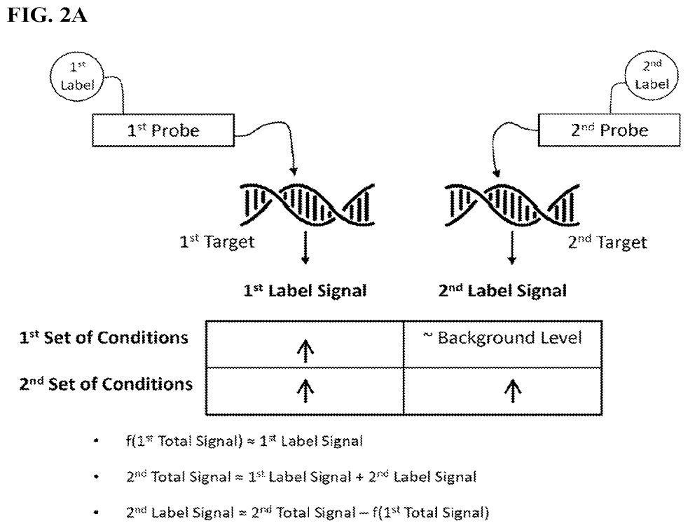

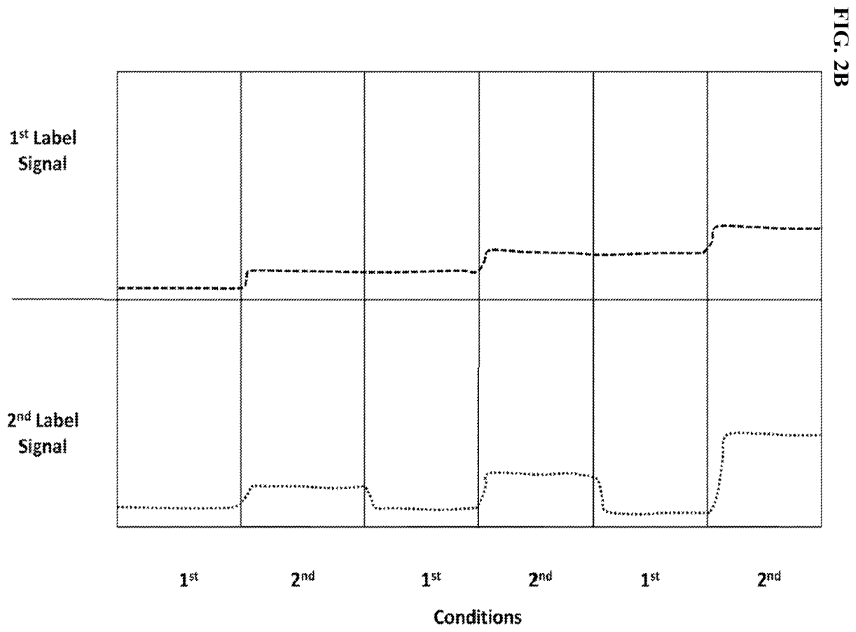

FIG. 2A is a schematic overview of a method for detecting multiple target nucleic acids within the same detection channel by providing different first and second probe types, varying the reaction mixture conditions, and measuring the resulting total signal at each set of conditions. As shown, a first probe is designed to specifically interact (“bind”) with a first target. The first probe includes a first label that can generate a first label signal. A second probe is designed to specifically interact with a second target that is different from the first target. The second probe includes a second label that can generate a second label signal.

In some embodiments, the first and second labels are the same. For example, the first and second labels may comprise the same fluorescent dye. In some embodiments, the first and second labels may be different, but are nonetheless designed to generate a substantially identical signal (e.g., substantially identical fluorescence). For example, the first and second labels may comprise dyes that are chemically distinct yet function to emit fluorescence signals with similar wavelengths. In some embodiments, the first and second label signals are measured using the same optical filter arrangement in the detection instrument. The first and second label signals are thus measured within the same detection channel.

The first probe and second probe may be provided in the same reaction mixture and allowed to specifically interact with any first and second target, respectively, in the reaction mixture. As shown, the reaction mixture is subjected to at least two different sets of reaction conditions. The first probe is designed such that the first label generates the first label signal, to a degree correlated with (e.g., proportional to) the amount of specific interaction between the first probe and first target, during both the first and second sets of conditions. In contrast, the second probe is designed such that the second label generates the second label signal, to a degree correlated with (e.g., proportional to) the amount of specific interaction between the second probe and second target, during the second set of conditions but not during the first set of conditions. Stated differently, under the first set of conditions, the first label signal is increased as a result of specific interaction of the first probe with the first target, but the second label signal is not increased as a result of specific interaction of the second probe with the second target, whereas under the second set of conditions, the second label signal is increased as a result of specific interaction of the second probe with the second target.

During the first set of conditions, the second label will not generate “substantial fluorescence,” and the second label signal will therefore not be substantially different from a background (i.e., baseline) level of fluorescence in the reaction mixture. That is, while there may be some non-zero level of signal generated by the second label during the first set of conditions, the second label signal will typically remain below a threshold value that separates background fluorescence from meaningful signal. This threshold may vary according to particular testing protocols and application needs, as discussed above.

In at least some embodiments, when both the first and the second targets are present in the reaction mixture, the second label signal will differ between the first and second sets of conditions to a greater degree than the first label signal will differ between the first and second sets of conditions. Thus, while the first label signal may differ somewhat between the first and second sets of conditions, this difference will typically be less than the difference in the second label signal between the first and second sets of conditions.

The difference in the manner the first and second label signals respond to the different sets of conditions can be exploited to enable separation of the first and second label signals even when they are within the same detection channel. For a given detection channel (e.g., for a given optical filter arrangement), the total signal during the first set of conditions (“the first total signal”) is measured and the total signal during the second set of conditions (“the second total signal”) is measured.

During the first set of conditions, the total signal will be substantially equal to the first label signal. That is, the first total signal is primarily composed of the first label signal, whereas contribution from the second label signal is negligible. During the second set of conditions, the total signal will include a combination of the first and second label signals. The first and second label signals can therefore be separately resolved based on the first and second total signals. For example, the first label signal can be determined based on the first total signal, and the second label signal can be resolved by subtracting the first total signal from the second total signal.

In some embodiments, the first label signal is equated directly to the first total signal. In other embodiments, the first label signal is determined as a function of the first total signal. In some embodiments, this function is a linear function. For example, as discussed above, the first label signal may differ slightly between the first and second sets of conditions even when the amount of first target has not changed. In certain applications, the first label signal under the second set of conditions may better correspond to standard curves that equate the first label signal to first target amounts. Estimating the first label signal as a function of the first total signal, rather than as directly equal to the first total signal, can therefore bring the calculated first label signal closer to what would be measured under the second set of conditions (i.e., without any interfering second label signal).

In some embodiments, the function for converting the first total signal to the first label signal is determined by comparing, in the absence of any second probe interacting with second target, the first label signal under the first set of conditions to the first label signal under the second set of conditions. The first label signal under the first set of conditions and under the second set of conditions often correlate to one another according to a linear function. Thus, a simple multiplier can be used to convert the first total signal to the first label signal. Once such a linear function is determined, it can be used in subsequent assays without necessarily requiring additional comparisons of the first label signal under the first set of conditions and under the second set of conditions in the absence of second probe with second target. In some embodiments, the function for converting the first total signal to the first label signal may be non-linear. In some embodiments, the function/correlation is determined where the number of cleaved probes is expected to be the same. That is, the first total signal of cycle “n” is compared to the second total signal of cycle “n−1.”

As described in greater detail below, the first probe and the second probe have different mechanisms of action that enable different signal responses, depending on the probe type, to the first and second sets of conditions. Beneficially, unlike certain prior approaches, the ability to resolve the separate signals respectively associated with each of the different probe types need not rely on different melting temperatures of the probes. Thus, although the first probe and second probe may have non-identical melting temperatures, they do not necessarily have substantially different melting temperatures to allow the separate signals to be effectively resolved. In some embodiments, for example, a melting temperature (Tₘ) (conventionally defined as the temperature at which 50% of the strands are in double-stranded form and 50% are single-stranded) of the first probe and a Tₘ of the second probe are within about 8° C., or about 6° C., or about 4° C., or about 2° C. of each other. In some embodiments, both probes are bound (e.g., hybridized) to their respective targets under the first or second set of conditions. Optionally, both probes are not substantially bound (e.g., hybridized) to their respective targets under the first or second set of conditions. In one example, both the first and second probes are substantially bound (e.g., hybridized) to their respective targets under the first set of conditions, whereas both the first and second probes are not substantially bound (e.g., hybridized) to their respective targets under the second set of conditions. In another example, both the first and second probes are not substantially bound (e.g., hybridized) to their respective targets under the first set of conditions, whereas both the first and second probes are substantially bound (e.g., hybridized) to their respective targets under the second set of conditions.

FIG. 2B is a graph showing signal response over time for the method outlined in FIG. 2A when the reaction mixture is cycled between the first set of reaction conditions and the second set of reaction conditions and when both the first and second targets are present in the reaction mixture. The cycling of conditions may comprise, for example, thermal cycling in a nucleic acid amplification reaction, where the first set of reaction conditions represents a denaturation step at a first temperature of the thermal cycling and the second set of reaction conditions includes an annealing and/or extension step (“annealing/extension step”) at a second temperature (lower than the first) of the thermal cycling.

As shown, both the first label signal and the second label signal increase under the second set of reaction conditions. Under the first set of reaction conditions, the first label signal remains roughly the same as at the end of the previous cycle (though it may vary slightly, as discussed above), whereas the second label signal drops to a level similar to the baseline signal level.

As shown, both the first label signal and the second label signal cumulatively increase at each successive occurrence of the second set of conditions. This is a result of additional specific interaction in the reaction mixture between the first probe and the first target and additional specific interaction in the reaction mixture between the second probe and the second target. However, where the first label signal remains at a similar level when moving from the end of one cycle to the beginning of another (i.e., when moving from the second set of conditions at the end of a cycle to the first set of conditions at the beginning of a subsequent cycle), the second label signal returns to a level near baseline at the beginning of each cycle (i.e., at each occurrence of the first set of conditions).

While embodiments described herein primarily focus on intra-channel multiplexing, the disclosed intra-channel multiplexing may be combined with inter-channel multiplexing to further increase the plexy of the assay. For example, an assay may be designed with multiple different dyes (and thus with multiple different detection channels), where two or more of the different channels each include multiple detectable signals that can be resolved using the methods described herein.

### V. Cleavable and Non-Cleavable Probes

In embodiments, the first probe is a “cleavable” probe. The first probe may be designed such that the first label is detached from the first probe (and released from a corresponding quencher, for example) as a result of hybridization of the first probe to the first target. Once released, the first label therefore continues to contribute to the total signal in the reaction mixture. The first probe may be a TaqMan probe, for example, which undergoes cleavage as a result of 5′ to 3′ exonuclease activity of DNA polymerase during extension of the target molecule to which the probe is hybridized. TaqMan probes are described in U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and 7,445,900, all of which are hereby incorporated herein by reference.

In embodiments, the second probe is a “non-cleavable” probe. The label of a non-cleavable probe is intended to remain associated with the probe throughout the assay, and to vary in the level of generated signal according to probe configuration rather than release of the label. The second probe may be an EF probe, for example, which quenches the label when in a single stranded configuration but allows signal when incorporated into a double stranded molecule.

In embodiments, the second probe has the formula:

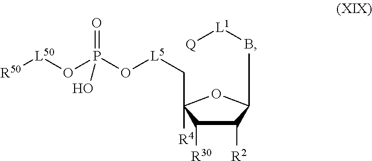

or a salt thereof, wherein Q is a quencher moiety (e.g., internal quencher moiety) moiety having the formula:

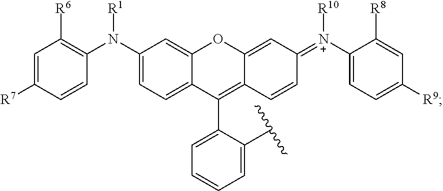

B is a divalent nucleobase; L¹ is a divalent linker; L⁵ is a divalent oligonucleotide linker comprising from 4 to 40 nucleotides; L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R⁵⁰ is the second label; R³⁰ is —OR^{30A}; R^{30A} is a monovalent oligonucleotide moiety; R² is hydrogen or —OR^{2A}; R⁴ is hydrogen or unsubstituted methyl, or R² and R⁴ substituents are joined to form a substituted or unsubstituted heterocycloalkyl; R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl; R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹ and R⁶ may be joined to form a substituted or unsubstituted heterocycloalkyl or substituted or unsubstituted heteroaryl; and R⁸ and R¹⁰ may be joined to form a substituted or unsubstituted heterocycloalkyl or substituted or unsubstituted heteroaryl; and R^{2A} and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, Q, B, L¹, L⁵, L⁵⁰, R¹, R², R⁴, R⁶, R⁷, R⁸, R⁹, R¹⁰, R³⁰, and R⁵⁰ are as described herein, including in embodiments.

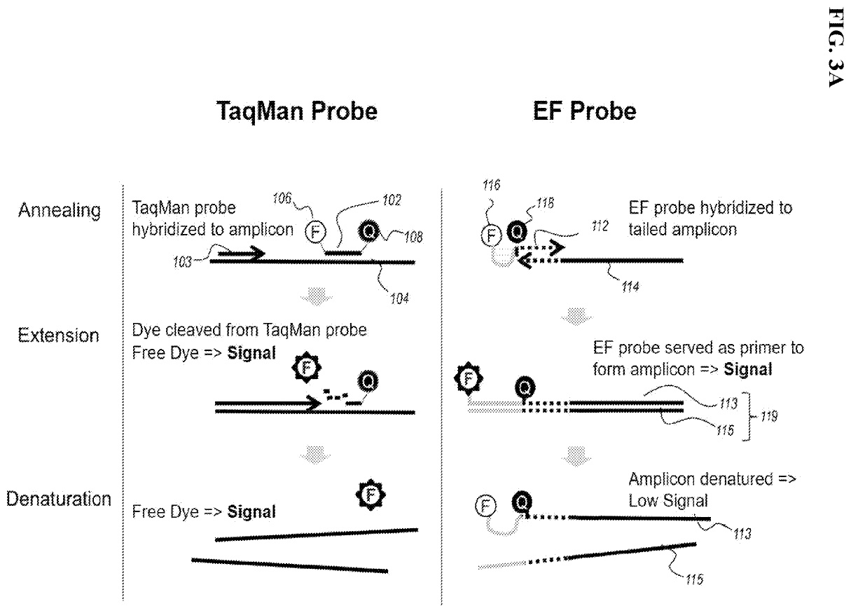

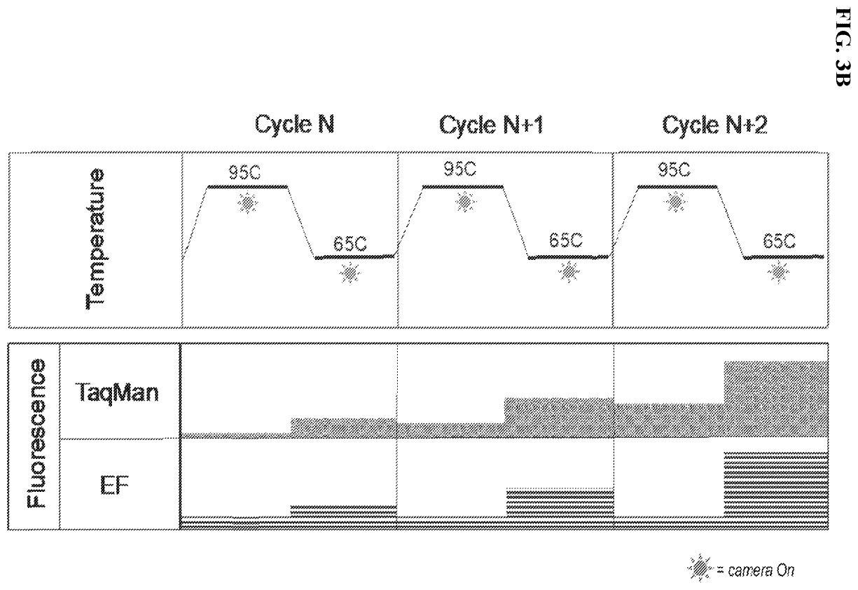

FIG. 3A illustrates activity of a cleavable TaqMan probe 102 and a non-cleavable EF probe 112 during annealing, extension, and denaturation steps of a thermal cycle. As shown, the TaqMan probe 102 hybridizes to its corresponding target 104 during the annealing stage. During extension of a primer 103 disposed upstream of the probe 102, the 5′ to 3′ exonuclease activity of a DNA polymerase cleaves the TaqMan probe label 106 from the remainder of the probe 102, thereby separating it from the corresponding TaqMan probe quencher 108. This leads to a corresponding increase in the fluorescence signal. During denaturation, the label 106 remains free within the reaction mixture solution and thus continues to contribute to the total fluorescence signal.

The EF probe 112 includes an EF probe label 116 and an EF probe quencher 118 which remain in proximity to one another while the probe 112 is in a single stranded configuration. The fluorescence signal from the label 112 thus remains substantially quenched while the EF probe is in a single stranded configuration. During the annealing and extension stages, the EF probe 112 hybridizes to its corresponding target template 114 and is extended to form an extended probe amplicon 113. Extension of target template 114 then forms the complement 115 of the extended probe amplicon 113. The resulting double stranded amplicon 119 forces the label 116 away from the quencher 118 to a distance sufficient to allow fluorescence emission. During denaturation, the extended probe amplicon 113 is separated from its complement 115. When returned to the single stranded configuration, the label 116 and quencher 118 are brought back into proximity and fluorescence is again quenched.

FIG. 3B is a graph showing the fluorescence signals from the TaqMan probes and the EF probes over time during thermal cycling of an amplification process. The temperatures of the thermal cycling may be varied according to particular application needs. As an example, the denaturation step may be carried out at a temperature of about 90° C. or more, such as about 95° C., and the annealing/extension step may be carried out at a lower temperature, such as about 50° C. to about 75° C. Here, the first set of reaction conditions corresponds to a denaturation step, while the second set of reaction conditions corresponds to an annealing/extension step.

While presently preferred embodiments cycle between a denaturation step and a combined annealing/extension step (i.e., the amplification process cycles between two target temperatures), other embodiments may include separate annealing and extension steps. In such embodiments, the temperature may be varied between the annealing and the extension steps. For example, the extension step may be carried out at a higher temperature than the annealing temperature. In embodiments, the amplification process cycles between two target temperatures for at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the cycles of the amplification process.

FIG. 3B shows that the fluorescence signal associated with the TaqMan probe increases during the extension phase and then remains at a similar level through the denaturation portion of the next cycle, whereas the fluorescence signal associated with the EF probe increases during the extension phase but decreases to baseline once the subsequent denaturation phase reaches the target denaturation temperature.

In embodiments, the first set of reaction conditions (e.g., the denaturation conditions) comprises a first measurement temperature at which the first label signal is measured, and the second set of reaction conditions (e.g., the annealing/extension conditions) comprises a second, different measurement temperature at which the first and second label signal is measured. In embodiments, the first and second measurement temperatures differ by at least about 10° C. or more, about 15° C. or more, about 20° C. or more, about 25° C. or more, or about 30° C. or more. The first measurement temperature may be the target denaturation temperature of about 90° C. or more, such as about 95° C., and the second measurement temperature may be the target annealing/extension temperature, such as about 50° C. to about 75° C.

### VI. EF Probe Template Formation

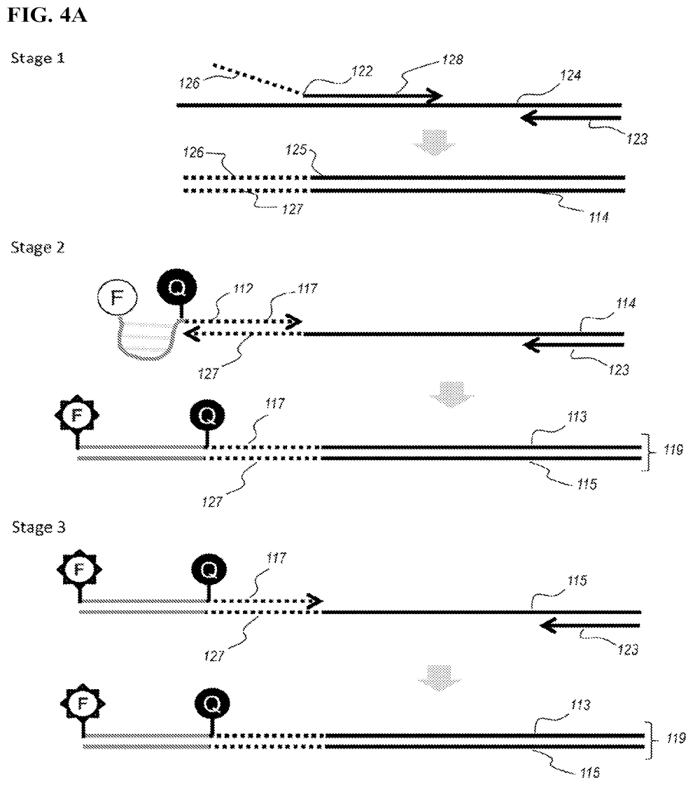

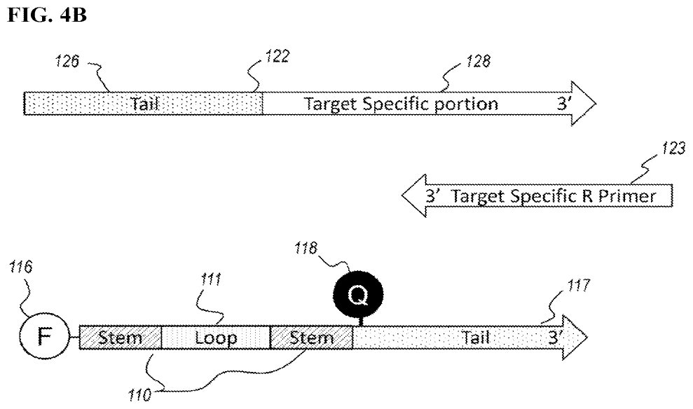

FIG. 4A illustrates a process of using a tailed primer 122, which is specific to a nucleic acid target 124, to form the target template 114 to which the EF probe 112 can hybridize. The tailed primer 122 includes a tail 126 and a target-specific portion 128. FIG. 4B illustrates an example of the tailed primer 122 as a forward primer, a target specific primer 123 paired with the tailed primer 122 as a reverse primer, and a more detailed view of the EF probe 112.

As shown, in a first stage, the target-specific portion 128 hybridizes to the target 124. Extension of the target-specific portion 128 forms a tailed amplicon 125. Primer 123, which is paired with the tailed primer 122, enables extension of the complement 114 of the tailed amplicon 125. It is this complement that forms the target template 114. As shown, the target template 114 includes a tail complement portion 127.

In a second stage, the EF probe 112 hybridizes to the target template 114 and amplification can continue as shown in FIG. 3A. As shown in FIG. 4A, the EF probe 112 includes a probe tail 117 that has substantial homology with the tail 126 and is therefore complementary to the tail complement portion 127 of the target template 114. Extension of probe 112 and target template 114 forms the double stranded amplicon 119. In the third stage, the primer 123, shown here paired with the tailed primer 122, may also function as the primer 123 that pairs with the EF probe 112 to enable formation of the double stranded amplicon 119, as shown in FIG. 3A.

As shown in FIG. 4B, the tail 126 can form the 5′ end of the tailed primer 122. The EF probe 112 can include a stem-loop portion, with stem portions 110 on either side of a loop portion 111, configured to form a stem-loop structure when the EF probe 112 is single stranded. For example, the label 116 may be located on one side of the stem-loop portion and the quencher 118 may be located on the opposite side of the stem-loop portion such that the label 116 and quencher 118 are brought into proximity when the stem-loop structure is formed but spaced farther apart when the EF probe 112 is constrained into a more linear configuration (e.g., when incorporated into a double stranded amplicon).

In the illustrated embodiment, the label 116 is located at or near the 5′ end of the EF probe 112 and the quencher 118 is located 3′ of the label 116. The positions of the label 116 and quencher 118 may be reversed in other embodiments. Preferably, as shown, the stem-loop portion is disposed 5′ of the probe tail 117 so that the stem-loop portion remains at the end of the amplicons resulting from extension of the EF probe 112, so that stem-loop structure formation (when single stranded) is less likely to be compromised.

In addition to or alternative to the EF probes described herein, some embodiments may include other labelled oligonucleotides that generate increased fluorescence upon being incorporated into a double stranded amplicon (relative to when in a single stranded state). For example, LUX™ primers include an internal fluorophore that is quenches by a hairpin structure located 5′ of the fluorophore. As with EF probes, a LUX™ primer provides increased fluorescence when incorporated into a double-stranded amplicon and the hairpin structure is linearized. Further, any of the primers or probes described herein may include one or more locked nucleic acids (LNAs) as are known in the art.

In some embodiments, the tailed primer 122 and the corresponding (non-tailed) primer 123 are provided at different concentrations. For example, the primer 123 may be provided at a higher concentration than the tailed primer 122. For example, the primer 123 may be provided at a concentration that is about 2× to about 30× the concentration of the tailed primer 122, or about 5× to about 25× the concentration of the tailed primer 122, or about 10× to about 20× the concentration of the tailed primer 122. Because the primer 123 can function to both (1) drive the formation of the target template 114 (as shown in FIG. 4A) and (2) drive the formation of the complement 115 of the extended probe amplicon 113 (as shown in FIG. 3A), providing it at a higher concentration than the corresponding tailed primer 122 can beneficially balance the reaction and help drive overall reaction efficiency.

In some embodiments, the EF probe 112 is provided at a concentration that is different from the concentration of the tailed primer 122 and/or the concentration of the primer 123. For example, the EF probe 112 may be provided at a concentration that is greater than the concentration of the tailed primer 122 and that is less than the concentration of primer 123. In some embodiments, the EF probe 112 is provided at a concentration that is about 2× to about 20× the concentration of the tailed primer 122, or about 3× to about 15× the concentration of the tailed primer 122. As discussed above, providing the primer 123 at a relatively higher concentration helps to drive the overall efficiency of the reaction. Providing the EF probe 112 at a concentration that is higher than the tailed primer 122, but not necessarily higher than the primer 123, pushes more of the associated amplification toward the EF probe 112 as opposed to the tailed primer 122, yet still allows the primer 123 to function as the primary driver of reaction efficiency.

In addition to or alternative to the “generic” EF probes that use a probe tail 117, other embodiments include and/or utilize EF probes with a target-specific portion rather than a probe tail 117. Such EF probes can directly hybridize to a target template and therefore do not need to follow the two-stage process shown in FIG. 4A for generating a target template 114 with a tail complement portion 127. In such embodiments, the probe tail 117 of the EF probe 112 is replaced with a target-specific portion that directly hybridizes to the target 124.

The process is otherwise similar to that shown in FIG. 3A. That is, after the EF probe is extended, a subsequent round of annealing/extension will extend the complement strand, forming the double stranded amplicon that separates the fluorophore and the quencher to allow for fluorescence signal generation.

FIG. 4C illustrates a three-stage thermal cycling method that may be utilized during an amplification process involving EF probes and optionally TaqMan probes. The amplification process shown in FIG. 4C may be used in conjunction with any of the other methods disclosed herein. The illustrated amplification process includes a first stage with a first target annealing/extension temperature, a second stage with a second, different annealing/extension temperature, and a third stage with a third annealing/extension temperature. In this embodiment, the third annealing/extension temperature is the same as the first annealing/extension temperature. Other embodiments may include a third annealing/extension temperature that is different from both the first and second annealing/extension temperatures.

The illustrated amplification process thus includes a denaturation temperature and multiple different annealing/extension temperatures that vary throughout the amplification process. Such an amplification process beneficially provides an initial stage (Stage 1) in which target template 114 is primarily formed, an intermediate stage (Stage 2) in which there is increased interaction between EF probes 112 and the target templates 114 to form the initial extended probe amplicons 113, and a later stage (Stage 3) in which amplification further involving the probe amplicons 113 and 115 can proceed.

As shown, the first annealing/extension temperature may be higher than the second annealing/extension temperature. The first series of denaturation and annealing/extension steps (in Stage 1) are cycled a greater number of times than the second series of denaturation and annealing/extension steps (in Stage 2). The third series of denaturation and annealing/extension steps (in Stage 3) may be cycled a greater number of times than the first series of denaturation and annealing/extension steps. The denaturation temperature may be the same for each series of denaturation and annealing/extension steps.

Stages 1 and 2 thus function as pre-loading stages that primarily generate target template 114 (in Stage 1) and then provide a lower annealing/extension temperature (in Stage 2), for at least one cycle, to allow increased interaction between the EF probes 112 and the target templates 114. Afterwards, multiple amplification cycles can then be carried out at the third annealing/extension temperature to drive amplification primarily involving the EF probes 112, primer 123 and/or their extended probe amplicons 113 and 115. Most of the amplification cycles are thus typically carried out during Stage 3.

### VII. Digital PCR

PCR is an in vitro method for the enzymatic synthesis of specific DNA sequences using two oligonucleotide primers that hybridize to opposite strands and flank the region of interest in target DNA. A repetitive series of reaction steps involving template denaturation, primer annealing, and the extension of the annealed primers by DNA polymerase results in the exponential accumulation of a specific fragment whose termini are defined by the primers. PCR can selectively enrich a specific DNA sequence by several orders of magnitude.

Digital polymerase chain reaction (dPCR) is a specific type of PCR that can be used to directly quantify target nucleic acids. In dPCR, the reaction mixture is partitioned into many small reaction volumes (i.e., partitions) so that the target nucleic acid is in some, but not all, of the reaction volumes/partitions. The reaction volumes are subjected to thermal cycling, and the proportion of “positive” partitions that generate a signal (usually a fluorescence signal) indicative of the presence of the target is determined. Quantitation is based on application of Poisson statistics, using the number of negative/non-reactive reaction volumes and assuming a Poisson distribution to establish the number of initial copies that were distributed across all the reaction volumes.

The features and principles described herein with respect to other amplification processes are also generally applicable to dPCR processes. Thus, although the description of this section provides a more detailed disclosure of dPCR embodiments, the disclosure provided elsewhere herein is also applicable to dPCR embodiments. Embodiments that include dPCR may utilize a variety of partitioning mechanisms or devices as known in the art or as may be developed in the future. For example, some conventional dPCR systems utilize a plurality of droplets encapsulated by an oil phase to form the plurality of partitions/reaction volumes. Other embodiments may utilize an array of microchambers. As example of such a system is the QuantStudio Absolute Q system available from Thermo Fisher Scientific, which uses a microfluidic array plate to perform the compartmentalizing/partitioning of sample.

In some dPCR embodiments, the reaction mixture is fully formed prior to partitioning into the plurality of reaction volumes. In alternative embodiments, one or more components of the reaction mixture may be pre-loaded onto or into the reaction volumes. For example, probes and/or primers may be coated onto the walls of microchambers, and the sample and/or other components of the reaction mixture are then added to the microchambers to form a plurality of reaction mixtures in each of the reaction volumes.

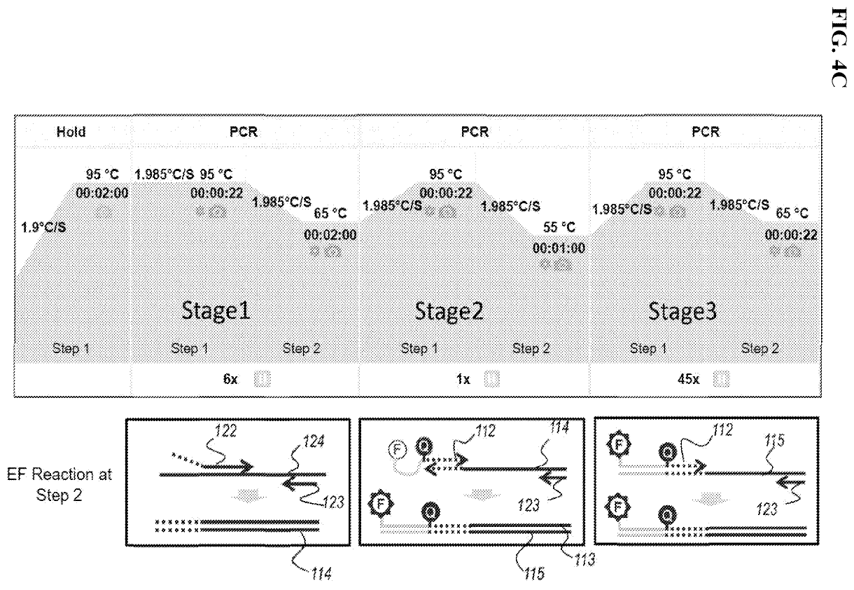

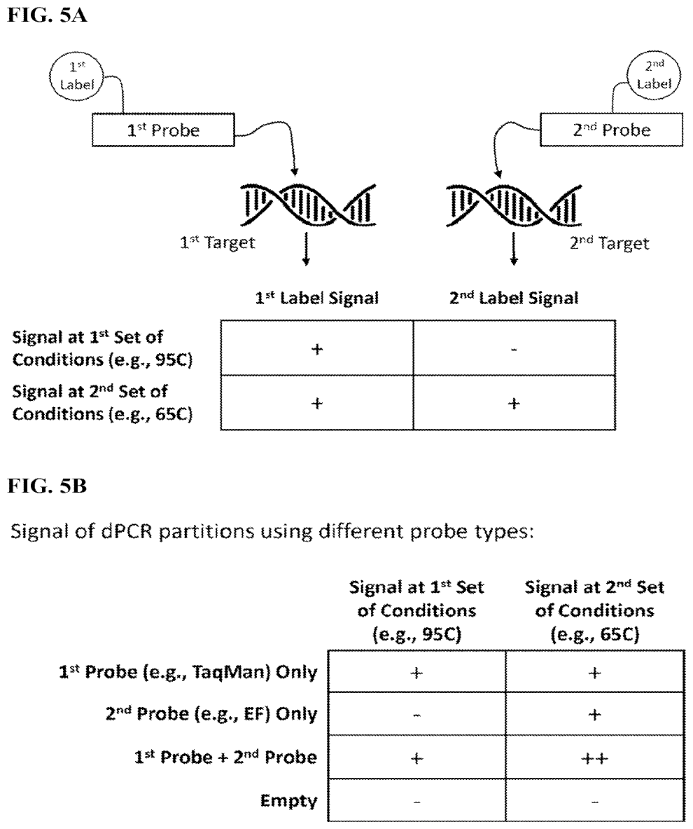

FIG. 5A is a schematic overview of a method for detecting multiple target nucleic acids within the same detection channel using a dPCR process. Whereas embodiments including real-time/quantitative PCR (qPCR) typically monitor amplification during the reaction, dPCR typically involves an end-point measurement to count and determine the number of “positive” partitions. Accordingly, FIG. 5A illustrates that under both the first set of conditions (e.g., denaturation conditions such as about 95° C.) and the second set of conditions (e.g., annealing/extension conditions such as about 65° C.) a partition in which the first probe and first target are present will show as positive (+) at the conclusion of the reaction. On the other hand, a partition in which the second probe and second target are present will show as negative (−) under the first set of conditions, but positive (+) under the second set of conditions.

This is similar to the schema illustrated in FIG. 2A, and FIG. 5A shows that the same principles can be applied to end-point measurements such as in dPCR. As described in other embodiments, the first probe may be a cleavable probe such as a TaqMan probe, and the second probe may be a non-cleavable probe such as an EF probe.

FIG. 5B illustrates how the signal (e.g., at an end-point measurement) for a dPCR partition can vary depending on whether the first probe, second probe, or both were active within the partition during the reaction. As shown, if only the first probe provides a signal, the partition will be positive (+) at both the first and second set of conditions. If only the second probe provides a signal, the partition will be negative (−) at the first set of conditions and positive (+) at the second set of conditions. If both the first and second probes provide a signal, the partition will be positive (+) at the first set of conditions and highly positive (++) at the second set of conditions. Of course, if neither probe types provide a signal, the partition will be negative (−) at either set of conditions.

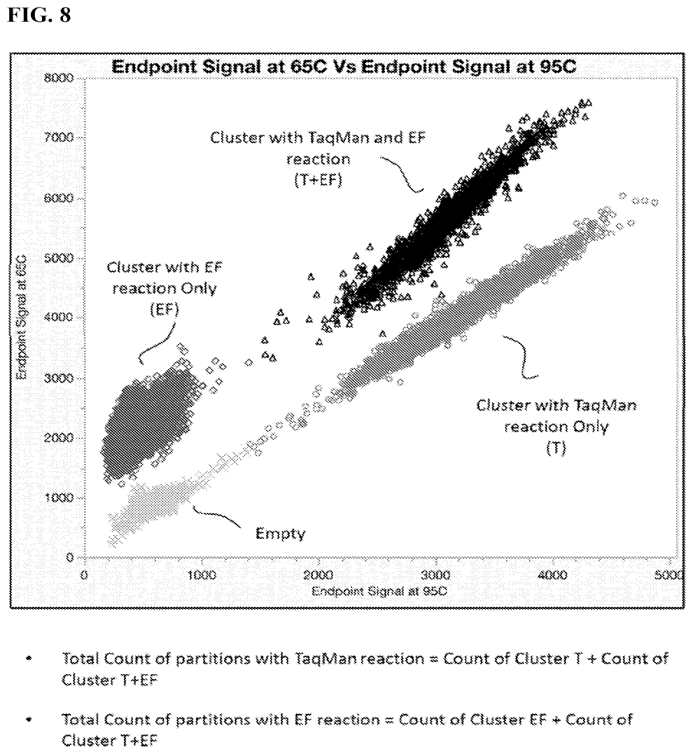

The total count of partitions that are positive for the first probe (and thus estimated as positive for the first target) is determined by counting the number of partitions that are positive (+) or highly positive (++) at both sets of conditions. The total count of partitions that are positive for the second probe (and thus estimated as positive for the second target) is determined by counting (i) the number of partitions that are positive (+) at the second set of conditions but negative (−) at the first set of conditions, and adding it to (ii) the number of partitions that are highly positive (++) at the second set of conditions. These partition counts may be calculated or estimated by plotting the end-point signal at the first set of conditions against the end-point signal at the second set of conditions and identifying clusters. See, for instance, the plot of FIG. 8, described in more detail in the Examples section below.

Concentrations of the first and second target in the sample may then be estimated using standard dPCR techniques.

Accordingly, a method for determining the presence of and/or amount of multiple targets using the intra-channel multiplexing features described herein in a dPCR application comprises the steps of: preparing a reaction mixture comprising a first probe type (e.g., TaqMan probe) and a second probe type (e.g., an EF probe), targeted to respective first and second nucleic acid targets; loading/partitioning a sample into a plurality of reaction volumes; measuring an end-point signal of the reaction volumes at a first set of reaction conditions (e.g., denaturation conditions such as about 95° C.); measuring an end-point signal of the reaction volumes at a second set of reaction conditions (e.g., annealing/extension conditions such as about 65° C.); categorizing the reaction volumes according to measured signal properties at the end-point measurements; determining or estimating a count for each probe type (i.e., a count of reaction volumes in which the first probe type was active and a count of reaction volumes in which the second probe type was active) based on the categorized reaction volumes; and determining or estimating the presence and/or amount of the first and second nucleic acid targets based on the counts.

### VIII. Additional Label/Dye Details

Exemplary detectable labels that may be utilized with the embodiments described herein include, for example:
Fluoresceins (e.g., 5-carboxy-2,7-dichlorofluorescein, 5-Carboxyfluorescein (5-FAM), 6-JOE, 6-carboxyfluorescein (6-FAM), VIC, FITC, 6-carboxy-4′,5′-dichloro-2′,7′-dimethoxy-fluorescein (JOE)), 5 and 6-carboxy-1,4-dichloro-2′,7′-dichloro-fluorescein (TET), 5 and 6-carboxy-1,4-dichloro-2′,4′,5′,7′-tetra-chlorofluorescein, HEX, PET, NED, Oregon Green (e.g. 488, 500, 514));
Pyrenes; (e.g. Cascade Blue; Alexa Fluor 405);
Coumarins; (e.g. Pacific Blue, Atto 425, Alexa Fluor 350, Alexa Fluor 430);
Cyanine Dyes; (e.g. Cy dyes such as Cy3, Cy3.18, Cy3.5, Cy5, Cy5.18, Cy5.5, Cy7); Rhodamines; (e.g., 110, 123, B, B 200, BB, BG, B extra, 5 and 6-carboxytetramethylrhodamine (5-TAMRA, 6-TAMRA), 5 and 6-Carboxyrhodamine 6G, Lissamine, Lissamine Rhodamine B, Rhod-2, ROX (6-carboxy-X-rhodamine), 5 and 6-ROX (carboxy-X-rhodamine), Sulphorhodamine B can C, Sulphorhodamine G Extra, 5 and 6 TAMRA (6-carboxytetramethyl-rhodamine), (TRITC), ABY, JUN, LIZ, RAD, RXJ, Texas Red; and Texas Red-X);
Alexa Fluor fluorophores (which is a broad class including many dye types such as cyanines) (e.g., Alexa 350, 405, 430, 488, 500, 514, 532, 546, 555, 568, 594, 610, 633, 635, 647, 660, 676, 680, 700, 750);
FRET donor/acceptor pairs (e.g., fluorescein/fluorescein, fluorescein/rhodamine, fluorescein/cyanine, rhodamine/cyanine, fluorescein/Alexa Fluor, Alexa Fluor/rhodamine); and other types of dyes known to those of skill in the art.

Fluorophore labels may be associated with quenchers such as dark fluorescent quencher (DFQ), black hole quenchers (BHQ), Iowa Black, QSY7, QSY21 quencher, Dabsyl and Dabcel sulfonate/carboxylate quenchers, and MGB-NFQ quenchers. Fluorophore labels may also include sulfonate derivatives of fluorescein dyes with SO3 instead of the carboxylate group, phosphoramidite forms of fluorescein, and/or phosphoramidite forms of Cy5, for example.

### IX. Additional Amplification Details

Amplified products resulting from use of one or more embodiments described herein can be generated, detected, and/or analyzed on any suitable platform. In embodiments, the nucleic acid targets may be single-stranded, double-stranded, or any other nucleic acid molecule of any size or conformation. The amplification processes described herein can include PCR (see, e.g., U.S. Pat. No. 4,683,202). In embodiments, the PCR is qPCR. In embodiments, the PCR is an end point PCR. In some embodiments, the PCR is dPCR.

In embodiments, the amplification process includes RT-PCR. A disclosed method may include, for example, subjecting the target nucleic acid to a reverse transcription reaction prior to amplification via PCR. In some embodiments, the amplification process includes one-step RT-PCR (e.g., in a single vessel or reaction volume) in which one or more reverse transcriptases are used in combination with one or more DNA polymerases.

Optionally, certain qPCR assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N8010560). Optionally, the different qPCR assays present in different wells of an array or plate can be dried or freeze-dried in situ and the array or plate can be stored or shipped prior to use. In some embodiments, the concepts described herein may be utilized in in situ hybridization applications not necessarily associated with PCR.

Other amplification methods, such as, e.g., loop-mediated isothermal amplification (“LAMP”), and other isothermal methods are also contemplated for use with the assay embodiments described herein.

The components described herein for enabling intra-channel multiplexing may be provided in a kit along with one or more additional components to enable an amplification process. Such components can include, for example, dNTPs, DNA polymerase, amplification buffers/reagents, master mix components as known in the art, and other components known in the art for enabling or assisting nucleic acid amplification.

### X. Computer System Implementation

In embodiments, at least a portion of the methods described herein may be implemented using one or more computer systems. In embodiments, the techniques discussed herein are represented in computer-executable instructions that may be stored on one or more hardware storage devices. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

In an example embodiment, a computer system comprises one or more processors, and a memory storing one or more instructions which, when executed by the one or more processors, cause the one or more processors to perform a process of: obtaining, at multiple time points during one or more cycles of an amplification process, fluorescence signal data associated with a composite fluorescence signal from at least a first probe type comprising a first fluorophore and a second probe type comprising a second fluorophore which has substantially overlapping spectral characteristics as said first fluorophore and/or generates a substantially identical signal, said first probe type and said second probe type differing in thermal and/or temporal properties; and determining, based at least partially on said fluorescence signal data associated with said composite fluorescence signal and thermal and/or temporal properties of one or more of said at least said first probe type and said second probe type, fluorescence signal data associated with a fluorescence signal from a given probe type of said at least said first probe type and said second probe type during said one or more cycles of said amplification process.

In embodiments, utilizing the fluorescence signal data associated with the composite fluorescence signal and the first fluorescence signal data as inputs for generating the fluorescence signal data associated with the fluorescence signal from the given probe type comprises: generating transformed first fluorescence signal data by applying a transformation (e.g., linear) to the first fluorescence signal data; and modifying the fluorescence signal data associated with the composite fluorescence signal with the transformed first fluorescence signal data to generate the fluorescence signal data associated with the fluorescence signal from the given probe type.

In embodiments, the one or more instructions, when executed by the one or more processors, further cause the one or more processors to perform a process of: quantifying a first target associated with the first probe type based upon at least the first fluorescence signal data; and quantifying a second target associated with the second probe type based upon at least the generated fluorescence signal data associated with the fluorescence signal from the given probe type.

Some embodiments include one or more computer-readable media storing one or more instructions which, when executed by one or more processors of at least one computing device, cause the one or more processors to perform the foregoing process or other computer-implemented process as described herein.

Systems for implementing the disclosed embodiments may include various components, such as, by way of non-limiting example, processor(s), storage, sensor(s), I/O system(s), communication system(s), and the like. The processor(s) may comprise one or more sets of electronic circuitries that include any number of logic units, registers, and/or control units to facilitate the execution of computer-readable instructions (e.g., instructions that form a computer program). Such computer-readable instructions may be stored within storage. The storage may comprise physical system memory and may be volatile, non-volatile, or some combination thereof. Furthermore, storage may comprise local storage, remote storage (e.g., accessible via communication system(s) or otherwise), or some combination thereof.

Furthermore, a system may comprise or be in communication with I/O system(s). I/O system(s) may include any type of input or output device such as, by way of non-limiting example, a touch screen, a mouse, a keyboard, a controller, a speaker and/or others, without limitation. For example, the I/O system(s) may include a display system that may comprise any number of display panels, optics, laser scanning display assemblies, and/or other components.

Disclosed embodiments may also include physical and other computer-readable media for carrying or storing computer-executable instructions and/or data structures. Such computer-readable media can be any available media that can be accessed by a general-purpose or special-purpose computer system. Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be in local and/or remote memory storage devices.

### XI. Embodiments

Embodiment 1. An oligonucleotide, or a salt thereof, having the formula:

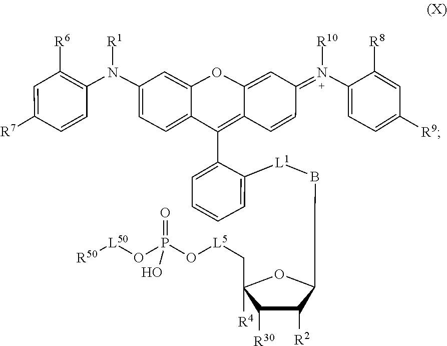

wherein
B is a divalent nucleobase;
L¹ is a divalent linker;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵⁰ is a detectable moiety;
R³⁰ is —OR^{30A} or —O—P(NR^{3B}R^{3C})—OR^{3A};
R^{30A} is hydrogen, a 3′ blocking moiety, or a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂ F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl;
R⁸ and R¹⁰ may be joined to form a substituted or unsubstituted heterocycloalkyl or substituted or unsubstituted heteroaryl; and
R^{2A}, R^{3A}, R^{3B}, R^{3C}, and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 2. The oligonucleotide of embodiment 1, having the formula:

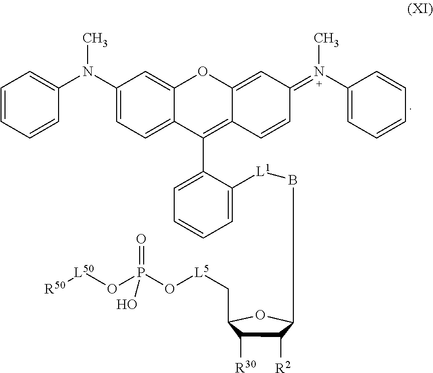

Embodiment 3. The oligonucleotide of embodiment 1, having the formula:

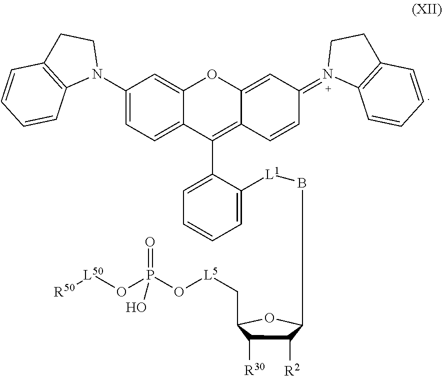

Embodiment 4. The oligonucleotide of embodiment 1, having the formula:

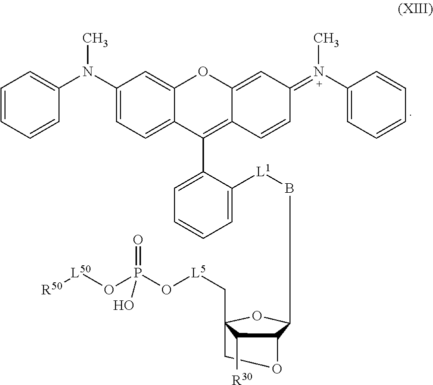

Embodiment 5. The oligonucleotide of embodiment 1, having the formula:

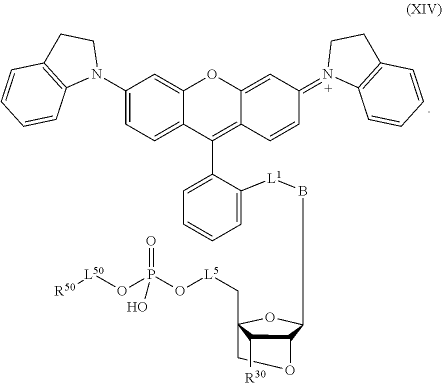

Embodiment 6. The oligonucleotide of one of embodiments 1 to 5, wherein B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof.

Embodiment 7. The oligonucleotide of one of embodiments 1 to 5, wherein B is a divalent cytosine or a derivative thereof, divalent guanine or a derivative thereof, divalent adenine or a derivative thereof, divalent thymine or a derivative thereof, or divalent uracil or a derivative thereof.

Embodiment 8. The oligonucleotide of embodiment 1, having the formula:

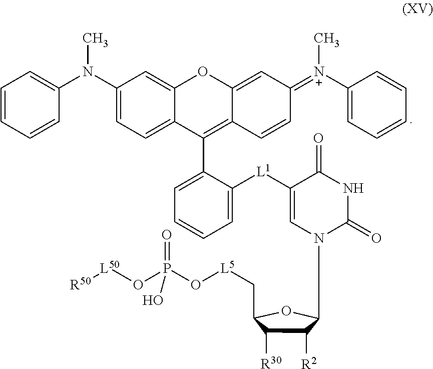

Embodiment 9. The oligonucleotide of embodiment 1, having the formula:

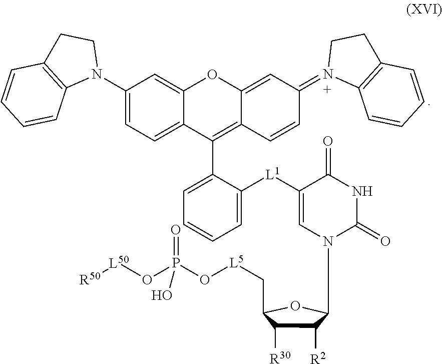

Embodiment 10 The oligonucleotide of embodiment 1, having the formula:

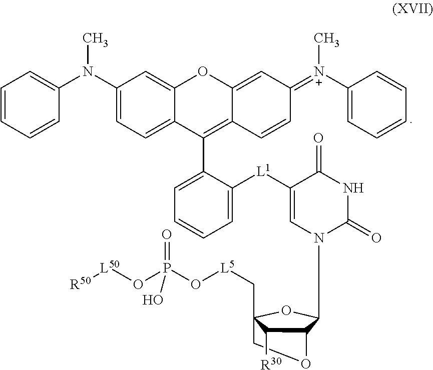

Embodiment 11. The oligonucleotide of embodiment 1, having the formula:

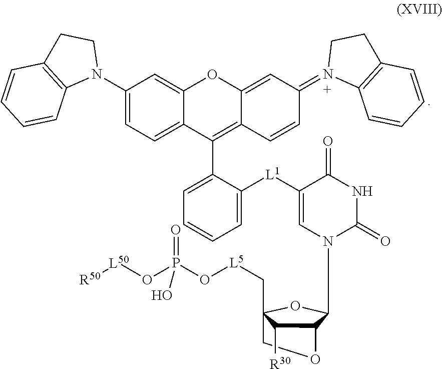

Embodiment 12. The oligonucleotide of one of embodiments 1 to 11, wherein L⁵ comprises from 11 to 30 nucleotides.

Embodiment 13. The oligonucleotide of one of embodiments 1 to 11, wherein L⁵ comprises from 19 to 23 nucleotides.

Embodiment 14. The oligonucleotide of one of embodiments 1 to 11, wherein L⁵ comprises from 4 to 14 nucleotides.

Embodiment 15. The oligonucleotide of one of embodiments 1 to 11, wherein L⁵ comprises from 6 to 12 nucleotides.

Embodiment 16. The oligonucleotide of one of embodiments 1 to 15, wherein the nucleotides are DNA nucleotides.

Embodiment 17. The oligonucleotide of one of embodiments 1 to 15, wherein the nucleotides are RNA nucleotides.

Embodiment 18. The oligonucleotide of one of embodiments 1 to 17, wherein L¹ is L¹⁰¹-L¹⁰²-L¹⁰³-L¹⁰⁴-L¹⁰⁵; and L¹⁰¹, L¹⁰², L¹⁰³, L¹⁰⁴, and L¹⁰⁵ are independently a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 19. The oligonucleotide of embodiment 18, wherein L¹⁰¹ is —S(O)₂—.

Embodiment 20. The oligonucleotide of one of embodiments 18 to 19, wherein L¹⁰² is an unsubstituted 3 to 8 membered heterocycloalkyl.

Embodiment 21. The oligonucleotide of one of embodiments 18 to 19, wherein L¹⁰² is an unsubstituted piperidinyl.

Embodiment 22. The oligonucleotide of one of embodiments 18 to 19, wherein L¹⁰² is

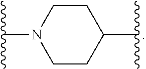 [SMILES: *C1CCN(*)CC1]

Embodiment 23. The oligonucleotide of one of embodiments 18 to 22, wherein L¹⁰³ is —C(O)NH—.

Embodiment 24. The oligonucleotide of one of embodiments 18 to 23, wherein L¹⁰⁴ is an unsubstituted C₁-C₁₀ alkylene, unsubstituted 2 to 6 membered heteroalkylene, or unsubstituted phenylene.

Embodiment 25. The oligonucleotide of one of embodiments 18 to 23, wherein L¹⁰⁴ is an unsubstituted n-hexylene,

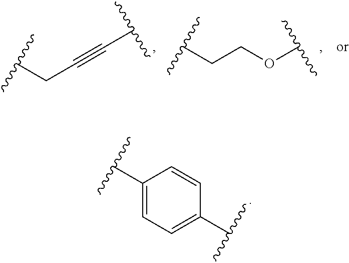 [SMILES: *C#CC* ; *CCO* ; *c1ccc(*)cc1]

Embodiment 26. The oligonucleotide of one of embodiments 18 to 25, wherein L¹⁰⁵ is an unsubstituted C₁-C₁₀ alkylene, substituted or unsubstituted 2 to 8 membered heteroalkylene, or unsubstituted 5 to 10 membered heteroarylene.

Embodiment 27. The oligonucleotide of one of embodiments 18 to 25, wherein L¹⁰⁵ is

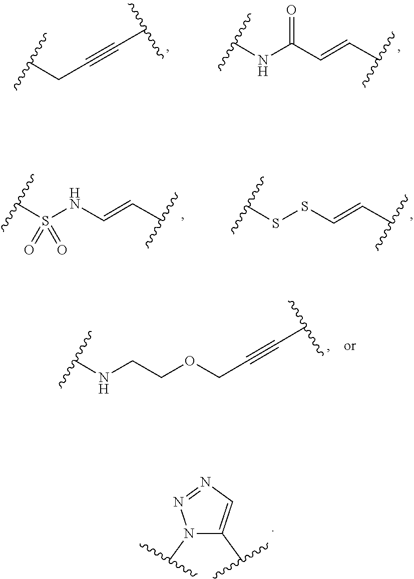 [SMILES: *C#CC* ; */C=C/C(=O)N* ; */C=C/NS(*)(=O)=O ; */C=C/SS* ; *C#CCOCCN* ; *c1cnnn1*]

Embodiment 28. The oligonucleotide of one of embodiments 1 to 17, wherein L¹ is

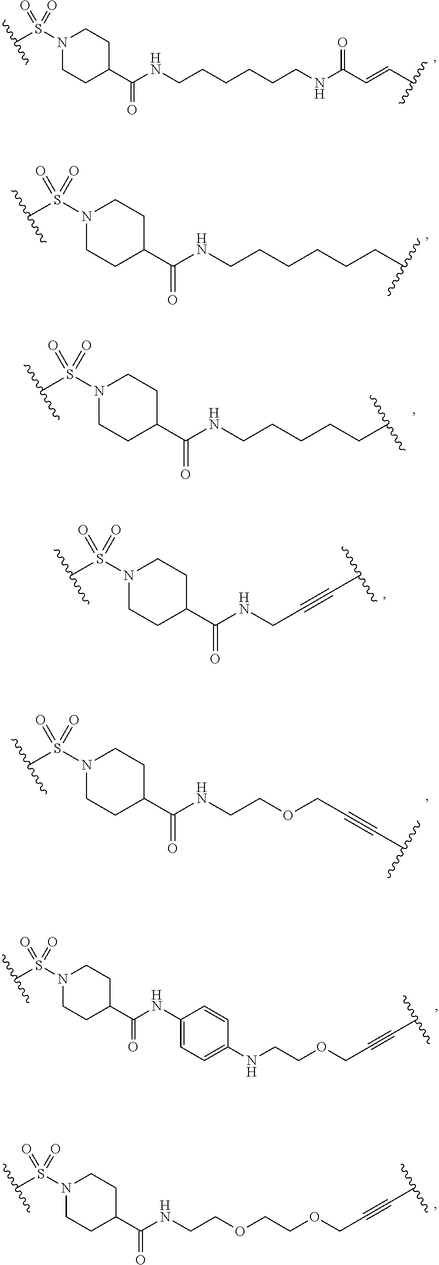

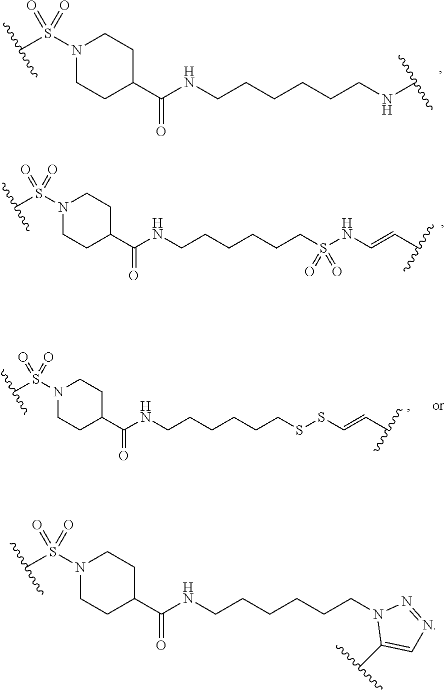 [SMILES: *NCCCCCCNC(=O)C1CCN(S(*)(=O)=O)CC1 ; */C=C/NS(=O)(=O)CCCCCCNC(=O)C1CCN(S(*)(=O)=O)CC1 ; */C=C/SSCCCCCCNC(=O)C1CCN(S(*)(=O)=O)CC1 ; *c1cnnn1CCCCCCNC(=O)C1CCN(S(*)(=O)=O)CC1]

Embodiment 29. The oligonucleotide of one of embodiments 1 to 28, wherein L⁵⁰ is a substituted 2 to 10 membered heteroalkylene.

Embodiment 30. The oligonucleotide of one of embodiments 1 to 28, wherein L⁵⁰ is

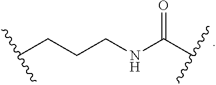 [SMILES: *CCCNC(*)=O]

Embodiment 31. The oligonucleotide of one of embodiments 1 to 30, wherein R⁵⁰ is a fluorescent moiety.

Embodiment 32. The oligonucleotide of embodiment 31, wherein R⁵⁰ is a monovalent form of FAM, a monovalent form of VIC, a monovalent form of ABY, a monovalent form of JUN, a monovalent form of AF647, a monovalent form of Cy5, a monovalent form of AF676, or a monovalent form of Cy5.5.

Embodiment 33. The oligonucleotide of one of embodiments 1 to 32, wherein R² is hydrogen or —OH.

Embodiment 34. The oligonucleotide of one of embodiments 1 to 32, wherein R² is hydrogen.

Embodiment 35. The oligonucleotide of one of embodiments 1 to 34, wherein R³⁰ is —OH.

Embodiment 36. The oligonucleotide of one of embodiments 1 to 34, wherein R³⁰ is

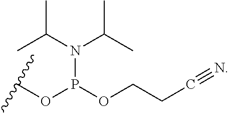 [SMILES: *OP(OCCC#N)N(C(C)C)C(C)C]

Embodiment 37. The oligonucleotide of one of embodiments 1 to 34, wherein the 3′ blocking moiety is a monovalent form of dideoxycytidine (3′ddC), a monovalent form of dideoxyadenosine (ddA), 3′ Inverted dT, 3′ amino modifier, a monovalent form of QSY7, a monovalent form of QSY21, a monovalent form of QSY9, a monovalent form of BHQ1, a monovalent form of BHQ2, a monovalent form of BHQ3, a monovalent form of Dabcyl, a monovalent form of Dabsyl, a monovalent form of Eclipse, a monovalent form of BBQ-650, a monovalent form of Iowa Black RQ, a monovalent form of Iowa Black FQ,

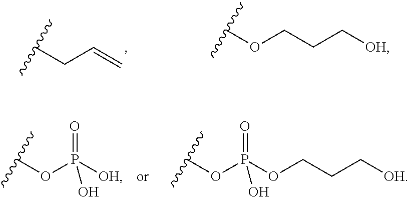

Embodiment 38. A compound, or a salt thereof, having the formula:

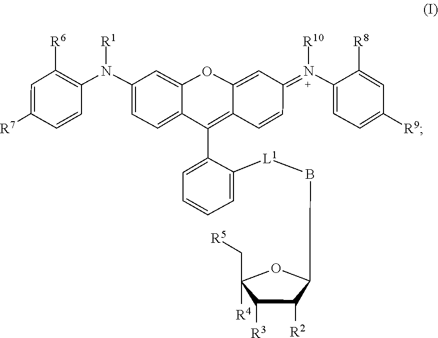

B is a divalent nucleobase;
L¹ is a divalent linker;
R² is hydrogen or —OR^{2A};
R³ is —OR^{3A} or —O—P(NR^{3B}R^{3C})—OR^{3A};
R⁴ is hydrogen or unsubstituted methyl, or R² and R⁴ substituents are joined to form a substituted or unsubstituted heterocycloalkyl;
R⁵ is —OR^{5A};
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl;
R⁸ and R¹⁰ may be joined to form a substituted or unsubstituted heterocycloalkyl or substituted or unsubstituted heteroaryl; and
R^{2A}, R^{3A}, R^{3B}, R^{3C}, R^{5A}, and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 39. The compound of embodiment 38, having the formula:

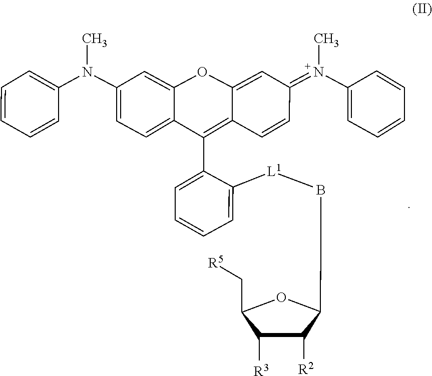

Embodiment 40. The compound of embodiment 38, having the formula:

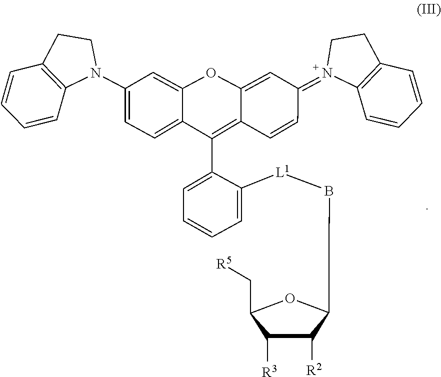

Embodiment 41. The compound of embodiment 38, having the formula:

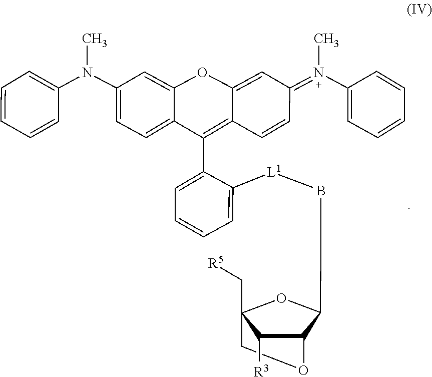

Embodiment 42. The compound of embodiment 38, having the formula:

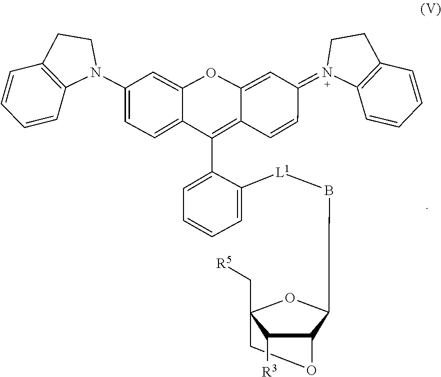

Embodiment 43. The compound of one of embodiments 38 to 42, wherein B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof.

Embodiment 44. The compound of one of embodiments 38 to 42, wherein B is a divalent cytosine or a derivative thereof, divalent guanine or a derivative thereof, divalent adenine or a derivative thereof, divalent thymine or a derivative thereof, or divalent uracil or a derivative thereof.

Embodiment 45. The compound of embodiment 38, having the formula:

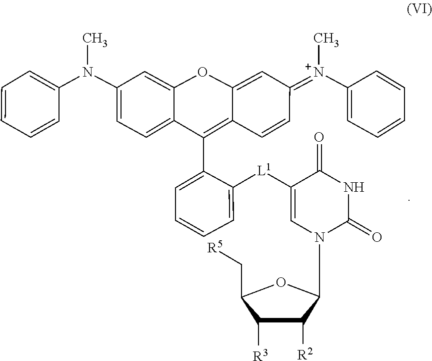

Embodiment 46. The compound of embodiment 38, having the formula:

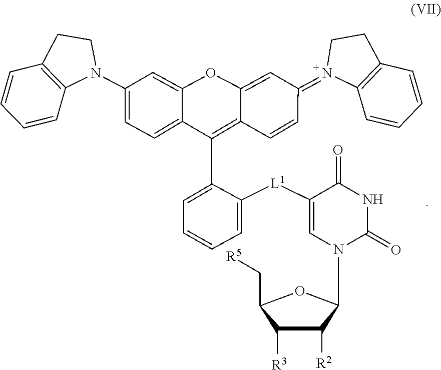

Embodiment 47. The compound of embodiment 38, having the formula:

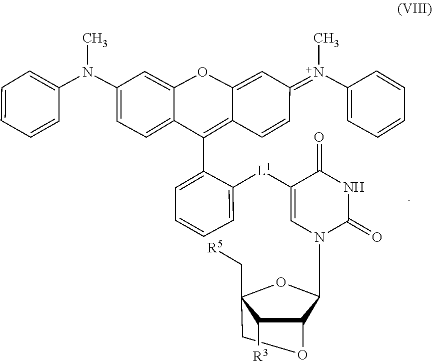

Embodiment 48. The compound of embodiment 38, having the formula:

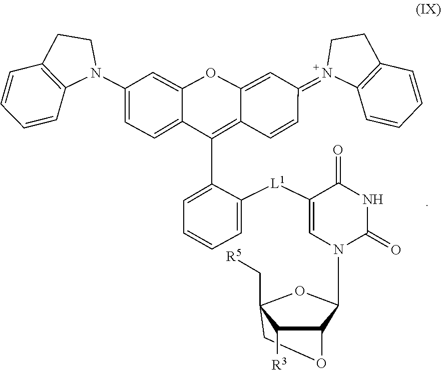

Embodiment 49. The compound of one of embodiments 38 to 48, wherein L¹ is L¹⁰¹-L¹⁰²-L¹⁰³-L¹⁰⁴-L¹⁰⁵; and L¹⁰¹, L¹⁰², L¹⁰³, L¹⁰⁴, and L¹⁰⁵ are independently a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 50. The compound of embodiment 49, wherein L¹⁰¹ is —S(O)₂—.

Embodiment 51. The compound of one of embodiments 49 to 50, wherein L¹⁰² is an unsubstituted 3 to 8 membered heterocycloalkyl.

Embodiment 52. The compound of one of embodiments 49 to 50, wherein L¹⁰² is an unsubstituted piperidinyl.

Embodiment 53. The compound of one of embodiments 49 to 50, wherein L¹⁰² is

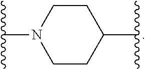 [SMILES: *C1CCN(*)CC1]

Embodiment 54. The compound of one of embodiments 49 to 53, wherein L¹⁰³ is —C(O)NH—.

Embodiment 55. The compound of one of embodiments 49 to 54, wherein L¹⁰⁴ is an unsubstituted C₁-C₁₀ alkylene, unsubstituted 2 to 6 membered heteroalkylene, or unsubstituted phenylene.

Embodiment 56. The compound of one of embodiments 49 to 54, wherein L¹⁰⁴ is an unsubstituted n-hexylene,

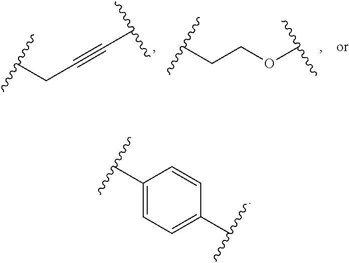 [SMILES: *C#CC* ; *CCO* ; *c1ccc(*)cc1]

Embodiment 57. The compound of one of embodiments 49 to 56, wherein L¹⁰⁵ is an unsubstituted C₁-C₁₀ alkylene, substituted or unsubstituted 2 to 8 membered heteroalkylene, or unsubstituted 5 to 10 membered heteroarylene.

Embodiment 58. The compound of one of embodiments 49 to 56, wherein L¹⁰⁵ is

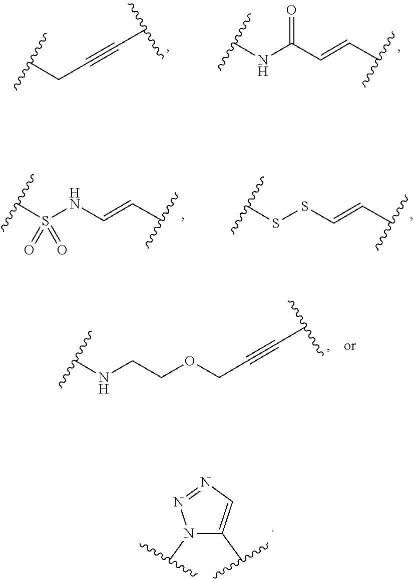 [SMILES: *C#CC* ; */C=C/C(=O)N* ; */C=C/NS(*)(=O)=O ; */C=C/SS* ; *C#CCOCCN* ; *c1cnnn1*]

Embodiment 59. The compound of one of embodiments 38 to 48, wherein L¹ is

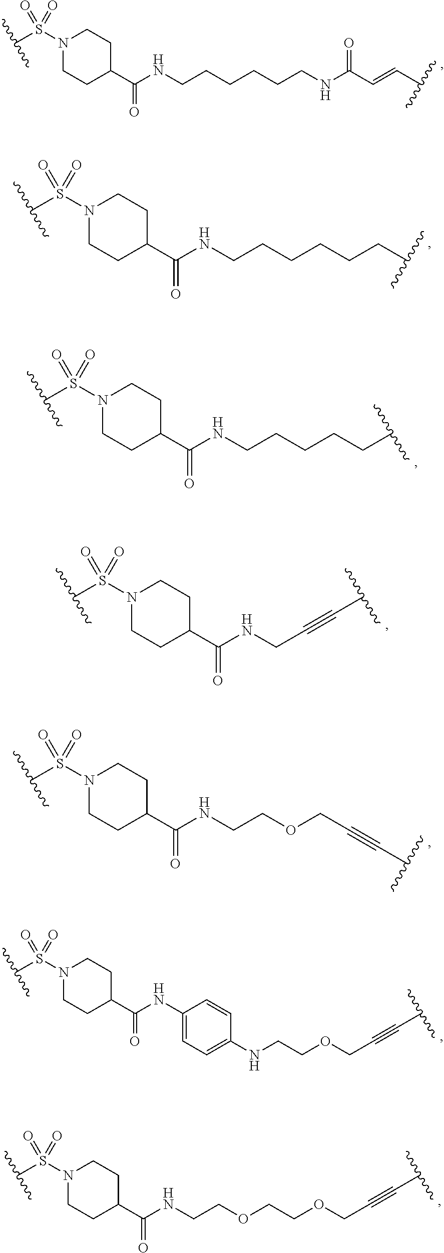

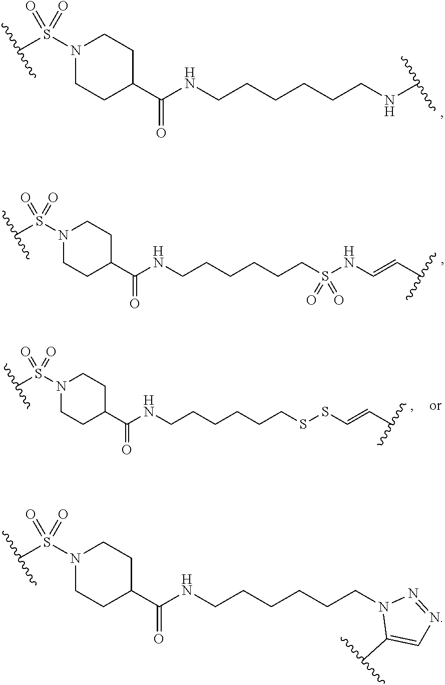 [SMILES: *NCCCCCCNC(=O)C1CCN(S(*)(=O)=O)CC1 ; */C=C/NS(=O)(=O)CCCCCCNC(=O)C1CCN(S(*)(=O)=O)CC1 ; */C=C/SSCCCCCCNC(=O)C1CCN(S(*)(=O)=O)CC1 ; *c1cnnn1CCCCCCNC(=O)C1CCN(S(*)(=O)=O)CC1]

Embodiment 60. The compound of one of embodiments 38 to 40 and 43 to 59, wherein R² is hydrogen or —OH.

Embodiment 61. The compound of one of embodiments 38 to 40 and 43 to 59, wherein R² is hydrogen.

Embodiment 62. The compound of one of embodiments 38 to 61, wherein R³ is —OH.

Embodiment 63. The compound of one of embodiments 38 to 61, wherein R³ is

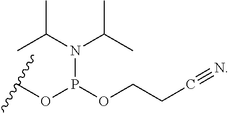 [SMILES: *OP(OCCC#N)N(C(C)C)C(C)C]

Embodiment 64. The compound of one of embodiments 38 to 63, wherein R^{5A} is hydrogen or substituted C₁-C₆ alkyl.

Embodiment 65. The compound of one of embodiments 38 to 63, wherein R^{5A} is dimethoxytrityl.

Embodiment 66. The compound of embodiment 38, having the formula:

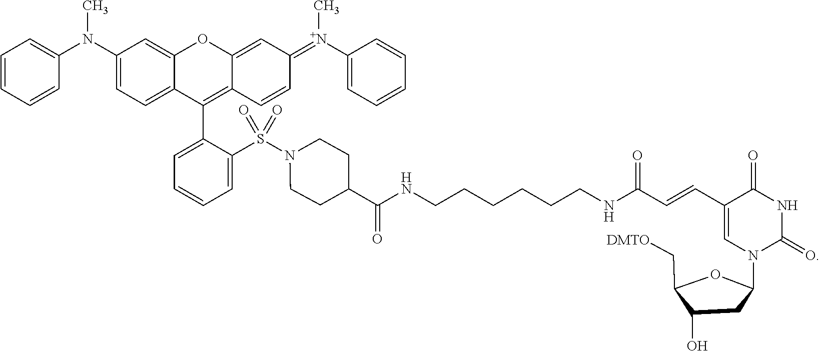

Embodiment 67. The compound of embodiment 38, having the formula:

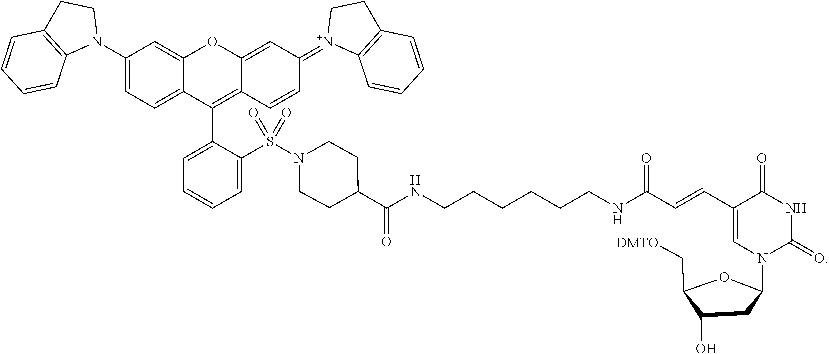

Embodiment 68. The compound of embodiment 38, having the formula:

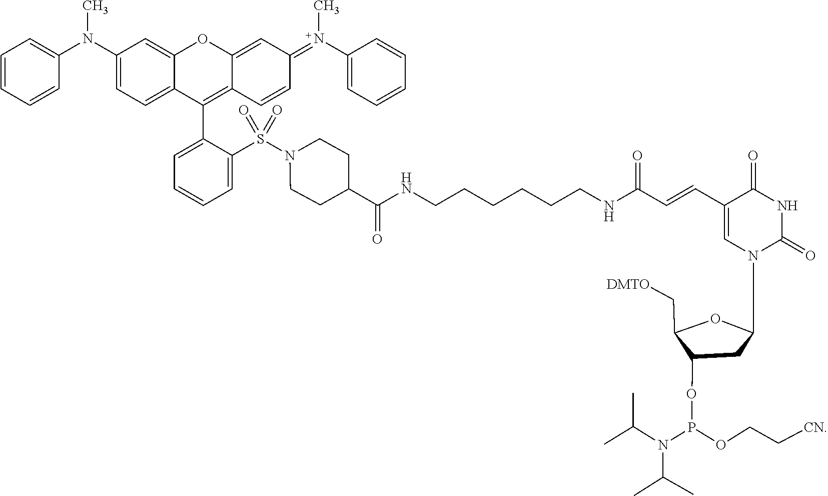

Embodiment 69. The compound of embodiment 38, having the formula:

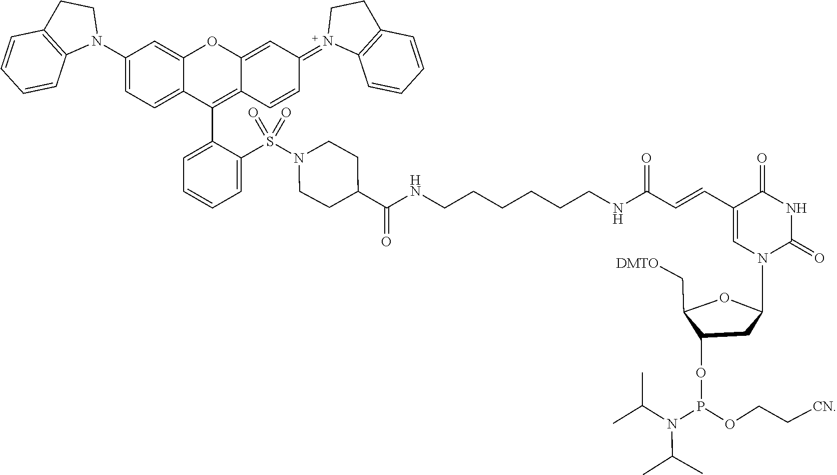

Embodiment 70. A method of forming a double-stranded nucleic acid, the method comprising contacting a target oligonucleotide with a nucleic acid probe comprising a quencher moiety, thereby forming the double-stranded nucleic acid; wherein the nucleic acid probe has the formula:

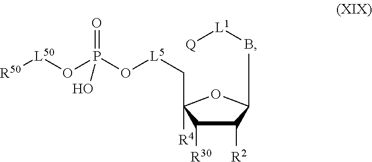

or a salt thereof, wherein
Q is the quencher moiety having the formula:

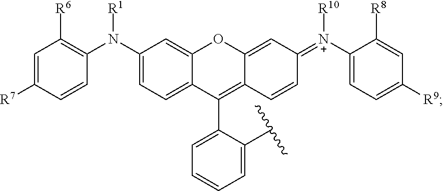

B is a divalent nucleobase;
L¹ is a divalent linker;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵⁰ is a detectable moiety;
R³⁰ is —OR^{30A};
R^{30A} is a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl;
R⁸ and R¹⁰ may be joined to form a substituted or unsubstituted heterocycloalkyl or substituted or unsubstituted heteroaryl; and
R^{2A} and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 71. The method of embodiment 70, wherein L⁵ comprises from 11 to 30 nucleotides.

Embodiment 72. The method of embodiment 70, wherein L⁵ comprises from 19 to 23 nucleotides.

Embodiment 73. The method of embodiment 70, wherein L⁵ comprises from 4 to 14 nucleotides.

Embodiment 74. The method of embodiment 70, wherein L⁵ comprises from 6 to 12 nucleotides.

Embodiment 75. The method of one of embodiments 70 to 74, wherein the nucleotides are DNA nucleotides.

Embodiment 76. The method of one of embodiments 70 to 74, wherein the nucleotides are RNA nucleotides.

Embodiment 77. The method of one of embodiments 70 to 76, further comprising contacting the double-stranded nucleic acid with a polymerase having exonuclease activity, thereby cleaving the quencher moiety from the detectable moiety.

Embodiment 78. The method of embodiment 77, further comprising detecting a signal generated by the detectable moiety.

Embodiment 79. The method of one of embodiments 70 to 78, wherein the detectable moiety is a fluorescent moiety.

Embodiment 80. The method of one of embodiments 78 to 79, further comprising amplifying the target oligonucleotide using PCR.

Embodiment 81. The method of embodiment 80, wherein the PCR is quantitative PCR.

Embodiment 82. The method of embodiment 80, wherein the PCR is digital PCR.

Embodiment 83. A method of detecting nucleic acids in a sample, comprising:
(i) providing a reaction mixture, the reaction mixture comprising:
   at least a portion of the sample,
   a first probe detectably labeled with a first label configured to generate a first fluorescence signal,
   a second probe detectably labeled with a second label configured to generate a second fluorescence signal,
   wherein the first and second probes have different sequences,
   wherein the first and second labels are identical and/or generate substantially identical fluorescence; and
   wherein the second probe has the formula:

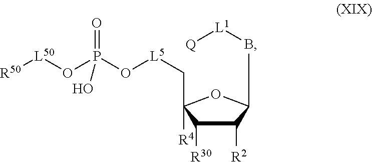

or a salt thereof, wherein
Q is a quencher moiety having the formula:

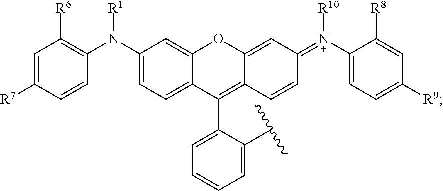

B is a divalent nucleobase;
L¹ is a divalent linker;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵⁰ is the second label;
R³⁰ is —OR^{30A};
R^{30A} is a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R′, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl;
R⁸ and R¹⁰ may be joined to form a substituted or unsubstituted heterocycloalkyl or substituted or unsubstituted heteroaryl; and
R^{2A} and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
(ii) subjecting the reaction mixture to an amplification process comprising a first set of reaction conditions and a second set of reaction conditions, the first set of reaction conditions being different than the second set of reaction conditions; and
(iii) determining the presence of the first and/or second nucleic acid targets in the sample by
   measuring a fluorescence signal during the first set of reaction conditions, the fluorescence signal during the first set of reaction conditions being correlated with specific interaction or lack of interaction between the first probe and a first nucleic acid target,
   measuring a fluorescence signal during the second set of reaction conditions, the fluorescence signal during the second set of reaction conditions being correlated with specific interaction or lack of interaction between the first probe and a first nucleic acid target and with specific or lack of interaction between the second probe and a second nucleic acid target, and
   estimating the presence and/or amount of the first nucleic acid target and second nucleic acid target.

Embodiment 84. A method of detecting nucleic acids in a sample, comprising:
(i) providing a reaction mixture, the reaction mixture comprising:
   at least a portion of the sample,
   a first probe detectably labeled with a first label configured to generate a first fluorescence signal that is indicative of the presence or absence of a first nucleic acid target,
   a second probe detectably labeled with a second label configured to generate a second fluorescence signal that is indicative of the presence or absence of a second nucleic acid target,
   wherein the first and second probes have different sequences,
   wherein the first and second labels are identical and/or generate substantially identical fluorescence; and
   wherein the second probe has the formula:

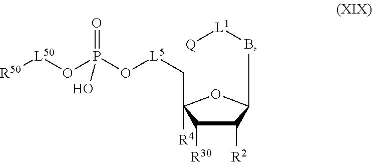

or a salt thereof, wherein
Q is a quencher moiety having the formula:

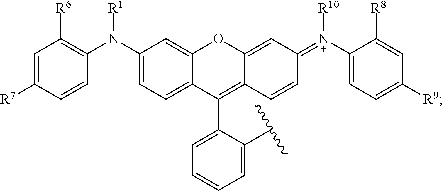

B is a divalent nucleobase;
L¹ is a divalent linker;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵⁰ is the second label;
R³⁰ is —OR^{30A};
R^{30A} is a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl; and
R⁸ and R¹⁰ may be joined to form a substituted or unsubstituted heterocycloalkyl or substituted or unsubstituted heteroaryl; and
R^{2A} and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
(ii) subjecting the reaction mixture to an amplification process comprising a first set of reaction conditions and a second set of reaction conditions, the first set of reaction conditions being different than the second set of reaction conditions; and
(iii) determining any presence and/or amount of the first and/or second nucleic acid targets in the reaction mixture by
   measuring during the first set of reaction conditions a first total fluorescence signal that comprises any first fluorescence signal if present and any second fluorescence signal if present,
   measuring during the second set of reaction conditions a second total fluorescence signal comprising any first fluorescence signal if present and any second fluorescence signal if present, and
   estimating the first fluorescence signal and/or second fluorescence signal based on the first and second total fluorescence signals.

Embodiment 85. The method of one of embodiments 83 to 84, wherein L⁵ comprises from 11 to 30 nucleotides.

Embodiment 86. The method of one of embodiments 83 to 84, wherein L⁵ comprises from 19 to 23 nucleotides.

Embodiment 87. The method of one of embodiments 83 to 84, wherein L⁵ comprises from 4 to 14 nucleotides.

Embodiment 88. The method of one of embodiments 83 to 84, wherein L⁵ comprises from 6 to 12 nucleotides.

Embodiment 89. The method of one of embodiments 83 to 88, wherein the nucleotides are DNA nucleotides.

Embodiment 90. The method of one of embodiments 83 to 88, wherein the nucleotides are RNA nucleotides.

Embodiment 91. The method of one of embodiments 83 to 90, wherein both the first and second probes are subjected to excitation at the same wavelength and/or both the first and second probes are subjected to excitation during detection of their respective first and second fluorescence signals.

Embodiment 92. The method of one of embodiments 83 to 91, wherein:
measuring the fluorescence signal during the first set of reaction conditions comprises measuring a combined signal comprising the first and second fluorescence signals during the first set of reaction conditions to obtain a first total fluorescence signal;
measuring the fluorescence signal during the second set of reaction conditions comprises measuring a combined signal comprising the first and second fluorescence signals during the second set of reaction conditions to obtain a second total fluorescence signal; and
estimating the presence and/or amount of the first nucleic acid target and the second nucleic acid target comprises estimating the first fluorescence signal and/or second fluorescence signal based on the first and second total fluorescence signals.

Embodiment 93. The method of embodiment 92, wherein the second fluorescence signal differs between the first and second set of reaction conditions to a greater degree than the first fluorescence signal differs between the first and second set of reaction conditions.

Embodiment 94. The method of one of embodiments 92 to 93, wherein
the first total fluorescence value comprises (i) fluorescence from first label that is free and unquenched within the reaction mixture and emitted as a result of the first label being cleaved following hybridization of the first probe to the first amplicon, and (ii) background fluorescence of the second label, and
the second total fluorescence value is based on (i) fluorescence from first label that is free and unquenched within the reaction mixture and emitted as a result of the first label being cleaved following hybridization of the first probe to the first amplicon, and (ii) fluorescence from the second label, above the background fluorescence of the second label, emitted as a result of hybridization of the second probe to the second amplicon.

Embodiment 95. The method of one of embodiments 92 to 94, further comprising:
calculating an amount of the first nucleic acid target based on the first fluorescent signal; and
calculating an amount of the second nucleic acid target based on the second fluorescent signal.

Embodiment 96. The method of one of embodiments 92 to 95, wherein when the first nucleic acid target is present in the reaction mixture, the first fluorescent signal is above a background level during both the first and second sets of reaction conditions.

Embodiment 97. The method of one of embodiments 92 to 96, wherein when the second nucleic acid target is present in the reaction mixture, the second fluorescent signal is above a background level during the second set of reaction conditions but not during the first set of reaction conditions.

Embodiment 98. The method of one of embodiments 93 to 96, wherein the first set of reaction conditions comprises a first measurement temperature at which the first fluorescence signal is measured, and the second set of reaction conditions comprises a second, different measurement temperature at which the second fluorescence signal is measured.

Embodiment 99. The method of embodiment 98, wherein the first and second measurement temperatures differ by at least about 10° C. or more, about 15° C. or more, about 20° C. or more, about 25° C. or more, or about 30° C. or more.

Embodiment 100. The method of one of embodiments 98 to 99, wherein at least one of the first or second measurement temperatures is a denaturation temperature at which DNA in the reaction mixture is denatured, such as about 90° C. or above.

Embodiment 101. The method of one of embodiments 83 to 100, wherein the reaction mixture is subjected to multiple amplification cycles each comprising the first and second set of reaction conditions.

Embodiment 102. The method of one of embodiments 83 to 101, wherein the amplification process comprises thermal cycling.

Embodiment 103. The method of embodiment 102, wherein the first set of reaction conditions comprises a denaturation step at a first temperature of the thermal cycling.

Embodiment 104. The method of embodiment 103, wherein the second set of reaction conditions comprises an annealing and/or extension step (“annealing/extension step”) at a second temperature of the thermal cycling, the second temperature being lower than the first temperature.

Embodiment 105. The method of one of embodiments 83 to 104, wherein the first probe is a cleavable probe.

Embodiment 106. The method of embodiment 105, wherein the first fluorescent signal increases when the cleavable probe is cleaved during an annealing/extension step.

Embodiment 107. The method of one of embodiments 105 to 106, wherein the first probe comprises a fluorophore and a quencher, and wherein the first probe is configured such that fluorescence from the fluorophore is quenched by the quencher until the probe is cleaved during an annealing/extension step of the amplification process.

Embodiment 108. The method of embodiment 107, wherein the first probe is a TaqMan probe.

Embodiment 109. The method of one of embodiments 83 to 108, wherein the second probe is a non-cleavable probe.

Embodiment 110. The method of embodiment 109, wherein L⁵ forms a stem-loop structure when the second probe is single-stranded.

Embodiment 111. The method of one of embodiments 109 to 110, wherein Q and R⁵⁰ are spaced apart from one another such that R⁵⁰ is quenched when the second probe is single stranded and unquenched when the second probe is incorporated into a double stranded amplicon.

Embodiment 112. The method of embodiment 111, wherein both Q and R⁵⁰ are disposed at or near the stem loop portion of the second probe.

Embodiment 113. The method of one of embodiments 83 to 112, wherein the reaction mixture further comprises: a first primer pair complementary to a first nucleic acid target of the nucleic acids or its complement, the first nucleic acid target being configured to generate a first amplicon with which the first probe can hybridize; and a second primer pair complementary to a second nucleic acid target of the nucleic acids or its complement, the second nucleic acid target being configured to generate a second amplicon with which the second probe can hybridize.

Embodiment 114. The method of embodiment 113, wherein the second primer pair comprises a primer with a tail.

Embodiment 115. The method of embodiment 114, wherein the tail forms the 5′ end of the primer with the tail.

Embodiment 116. The method of one of embodiments 114 to 115, wherein the second probe can hybridize to the tail or to its complement.

Embodiment 117. The method of one of embodiments 108 to 116, wherein the amplification process utilizes a series of thermal cycling steps that comprises at least three different target temperatures.

Embodiment 118. The method of embodiment 117, wherein the amplification process comprises a denaturation temperature and multiple different annealing/extension temperatures that vary throughout the amplification process.

Embodiment 119. The method of embodiment 118, wherein a first series of denaturation and annealing/extension steps are carried out at a first annealing/extension temperature, and wherein a second series of denaturation and annealing/extension steps are carried out at a second, different annealing/extension temperature.

Embodiment 120. The method of embodiment 119, wherein the first annealing/extension temperature is higher than the second annealing/extension temperature.

Embodiment 121. The method of one of embodiments 119 to 120, wherein the first series of denaturation and annealing/extension steps are cycled a greater number of times than the second series of denaturation and annealing/extension steps.

Embodiment 122. The method of one of embodiments 119 to 121, wherein the amplification process further comprises a third series of denaturation and annealing/extension steps carried out using a third annealing/extension temperature.

Embodiment 123. The method of embodiment 122, wherein the third annealing/extension temperature is the same as the first annealing/extension temperature.

Embodiment 124. The method of one of embodiments 122 to 123, wherein the third series of denaturation and annealing/extension steps are cycled a greater number of times than the first series of denaturation and annealing/extension steps.

Embodiment 125. The method of one of embodiments 119 to 124, wherein the denaturation temperature is the same for each series of denaturation and annealing/extension steps.

Embodiment 126. The method of one of embodiments 123 to 125, wherein the second primer pair comprises the tailed primer and a non-tailed primer, and wherein the tailed primer and non-tailed primer are provided at different concentrations.

Embodiment 127. The method of embodiment 126, wherein the non-tailed primer is provided at a greater concentration than the tailed primer.

Embodiment 128. The method of embodiment 127, wherein the non-tailed primer is provided at a concentration that is about 2× to about 30× the concentration of the tailed primer, or about 5× to about 25× the concentration of the tailed primer, or about 10× to about 20× the concentration of the tailed primer.

Embodiment 129. The method of one of embodiments 126 to 128, wherein the second probe is provided at a concentration that is different from the concentration of the tailed primer and the concentration of the non-tailed primer.

Embodiment 130. The method of embodiment 129, wherein the second probe is provided at a concentration that is greater than the concentration of the tailed primer.

Embodiment 131. The method of one of embodiments 129 to 130, wherein the second probe is provided at a concentration that is less than the concentration of the non-tailed primer.

Embodiment 132. The method of one of embodiments 129 to 131, wherein the second probe is provided at a concentration that is about 2× to about 10× the concentration of the tailed primer, or about 3× to about 7.5× the concentration of the tailed primer.

Embodiment 133. The method of one of embodiments 83 to 132, wherein a melting temperature (Tₘ) of the first probe and a Tₘ of the second probe are within about 8° C., or about 6° C., or about 4° C., or about 2° C. of each other.

Embodiment 134. The method of one of embodiments 83 to 133, wherein the amplification process cycles between two target temperatures for multiple cycles of the amplification process.

Embodiment 135. The method of embodiment 134, wherein the amplification process cycles between two target temperatures for at least 5% of, at least 10% of, at least 15% of, at least 20% of, at least 25% of, at least 30% of, at least 35% of, at least 40% of, at least 45% of, at least 50% of, at least 55% of, at least 60% of, at least 65% of, at least 70% of, at least 75% of, at least 80% of, at least 85% of, at least 90% of, or at least 95% of the cycles of the amplification process.

Embodiment 136. The method of one of embodiments 83 to 135, wherein the reaction mixture is added to or formed within a plurality of reaction volumes, and wherein the amplification process is a digital PCR (dPCR) process.

Embodiment 137. The method of embodiment 136, wherein measuring the fluorescence signal during the first set of reaction conditions comprises a first end-point measurement at the first set of reaction conditions, and wherein measuring the fluorescence signal during the second set of reaction conditions comprises a second end-point measurement at the second set of reaction conditions.

Embodiment 138. The method of embodiment 137, wherein estimating the presence and/or amount of the first nucleic acid target and the second nucleic acid target comprises:
categorizing the plurality of reaction volumes according to measured signal at the first end-point measurement and according to measured signal at the second end-point measurement; and based on the categorizations, determining a count for reaction volumes in which the first probe showed activity and a count for reaction volumes in which the second probe showed activity.

Embodiment 139. A method of detecting nucleic acids in a sample, comprising:
providing a reaction mixture, the reaction mixture comprising
a primer pair complementary to a nucleic acid target or its complement for generating an amplicon, and
a non-cleavable probe configured to hybridize to the amplicon, the probe comprising a detectable label configured to provide a fluorescent signal that corresponds to an amount of generated amplicon,
subjecting the reaction mixture to an amplification process to generate the amplicons, wherein the label generates fluorescence without cleavage of the probe during the amplification process, and wherein the amplification process utilizes a series of thermal cycling steps that includes at least three different target temperatures; and
measuring the fluorescent signal from the probe;
wherein the probe has the formula:

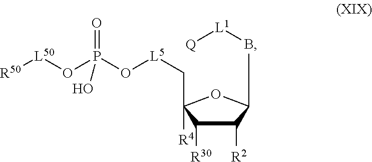

or a salt thereof, wherein
Q is a quencher moiety having the formula:

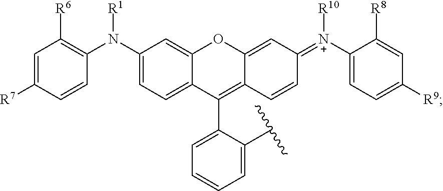

B is a divalent nucleobase;
L¹ is a divalent linker;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵⁰ is the detectable label;
R³⁰ is —OR^{30A};
R^{30A} is a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl;
R⁸ and R¹⁰ may be joined to form a substituted or unsubstituted heterocycloalkyl or substituted or unsubstituted heteroaryl; and
R^{2A} and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 140. The method of embodiment 139, further comprising quantitating an amount of the nucleic acid target based on the measured fluorescent signal.

Embodiment 141. The method of one of embodiments 139 to 140, wherein the probe comprises a stem-loop portion capable of forming a stem-loop structure when the probe is single stranded.

Embodiment 142. The method of one of embodiments 139 to 141, wherein Q and R⁵⁰ are spaced such that R⁵⁰ is quenched when the probe is single stranded but enabled when the probe is incorporated into a double stranded amplicon.

Embodiment 143. The method of embodiment 142, wherein both Q and R⁵⁰ are at or near the stem loop portion of the probe.

Embodiment 144. The method of one of embodiments 139 to 143, wherein the primer pair comprises a primer with a tail.

Embodiment 145. The method of embodiment 144, wherein the tail forms the 5′ end of the primer with the tail.

Embodiment 146. The method of one of embodiments 144 to 145, wherein the probe is configured to hybridize to the tail or to its complement.

Embodiment 147. The method of embodiment 146, wherein a 3′ portion of the probe is configured to hybridize to the tail or its complement.

Embodiment 148. The method of one of embodiments 139 to 147, wherein the amplification process comprises a denaturation temperature and multiple different annealing/extension temperatures that vary throughout the amplification process.

Embodiment 149. The method of embodiment 148, wherein a first series of denaturation and annealing/extension steps are carried out at a first annealing/extension temperature, and wherein a second series of denaturation and annealing/extension steps are carried out at a second, different annealing/extension temperature.

Embodiment 150. The method of embodiment 149, wherein the first annealing/extension temperature is higher than the second annealing/extension temperature.

Embodiment 151. The method of one of embodiments 149 to 150, wherein the first series of denaturation and annealing/extension steps are cycled a greater number of times than the second series of denaturation and annealing/extension steps.

Embodiment 152. The method of one of embodiments 149 to 151, wherein the amplification process further comprises a third series of denaturation and annealing/extension steps carried out using a third annealing/extension temperature.

Embodiment 153. The method of embodiment 152, wherein the third annealing/extension temperature is the same as the first annealing/extension temperature.

Embodiment 154. The method of one of embodiments 152 to 153, wherein the third series of denaturation and annealing/extension steps are cycled a greater number of times than the first series of denaturation and annealing/extension steps.

Embodiment 155. The method of one of embodiments 143 to 154, wherein the denaturation temperature is the same for each series of denaturation and annealing/extension steps.

Embodiment 156. The method of one of embodiments 144 to 155, wherein the primer pair comprises the tailed primer and a non-tailed primer, and wherein the tailed primer and non-tailed primer are provided at different concentrations.

Embodiment 157. The method of embodiment 156, wherein the non-tailed primer is provided at a greater concentration than the tailed primer.

Embodiment 158. The method of embodiment 157, wherein the non-tailed primer is provided at a concentration that is about 2× to about 30× the concentration of the tailed primer, or about 5× to about 25× the concentration of the tailed primer, or about 10× to about 20× the concentration of the tailed primer.

Embodiment 159. The method of one of embodiments 156 to 158, wherein the probe is provided at a concentration that is different from the concentration of the tailed primer and the concentration of the non-tailed primer.

Embodiment 160. The method of embodiment 159, wherein the probe is provided at a concentration that is greater than the concentration of the tailed primer.

Embodiment 161. The method of one of embodiments 159 to 160, wherein the probe is provided at a concentration that is less than the concentration of the non-tailed primer.

Embodiment 162. The method of one of embodiments 159 to 161, wherein the probe is provided at a concentration that is about 2× to about 10× the concentration of the non-tailed primer, or about 3× to about 7.5× the concentration of the non-tailed primer.

Embodiment 163. A method of detecting nucleic acids in a sample, comprising:
providing a reaction mixture, the reaction mixture comprising
   a primer pair targeted to a nucleic acid target for generating an amplicon, the primer pair comprising a tailed primer and a non-tailed primer provided at different concentrations, and
   a non-cleavable probe configured to hybridize to the amplicon, the probe comprising a detectable label configured to generate a fluorescent signal that corresponds to an amount of generated amplicon,
subjecting the reaction mixture to an amplification process to generate the amplicons, wherein the probe generates fluorescence without being cleaved during the amplification process; and
measuring the fluorescent signal from the probe;
wherein the probe has the formula:

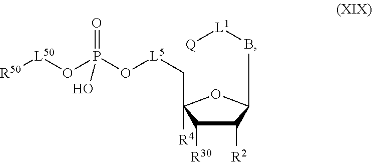

or a salt thereof, wherein
Q is a quencher moiety having the formula:

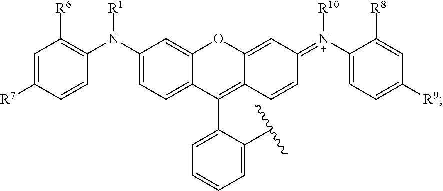

B is a divalent nucleobase;
L¹ is a divalent linker;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵⁰ is the detectable label;
R³⁰ is —OR^{30A};
R^{30A} is a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl;
R⁸ and R¹⁰ may be joined to form a substituted or unsubstituted heterocycloalkyl or substituted or unsubstituted heteroaryl; and
R^{2A} and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 164. The method of embodiment 163, wherein the non-tailed primer is provided at a greater concentration than the tailed primer.

Embodiment 165. The method of embodiment 164, wherein the non-tailed primer is provided at a concentration that is about 2× to about 30× the concentration of the tailed primer, or about 5× to about 25× the concentration of the tailed primer, or about 10× to about 20× the concentration of the tailed primer.

Embodiment 166. The method of one of embodiments 163 to 165, wherein the probe is provided at a concentration that is different from the concentration of the tailed primer and the concentration of the non-tailed primer.

Embodiment 167. The method of embodiment 166, wherein the probe is provided at a concentration that is greater than the concentration of the tailed primer.

Embodiment 168. The method of one of embodiments 166 to 167, wherein the probe is provided at a concentration that is less than the concentration of the non-tailed primer.

Embodiment 169. The method of one of embodiments 166 to 168, wherein the probe is provided at a concentration that is about 2× to about 10× the concentration of the non-tailed primer, or about 3× to about 7.5× the concentration of the non-tailed primer.

Embodiment 170. A method of detecting the presence or amount of a first and/or second target in a reaction mixture, comprising:
including a first probe and a second probe in the reaction mixture, wherein the first probe can specifically interact with a first target and comprises a first label that can produce a first detectable signal, and the second probe can specifically interact with a second target and comprises a second label that can produce a second detectable signal;
wherein the second probe has the formula:

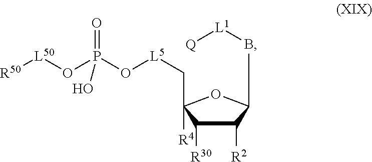

or a salt thereof, wherein
Q is a quencher moiety having the formula:

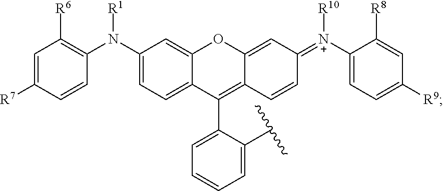

B is a divalent nucleobase;
L¹ is a divalent linker;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵⁰ is the second label;
R³⁰ is —OR^{30A};
R^{30A} is a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl; and
R⁸ and R¹⁰ may be joined to form a substituted or unsubstituted heterocycloalkyl or substituted or unsubstituted heteroaryl; and
R^{2A} and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
allowing specific interaction of the first and second probe with any first and second target, respectively, in the reaction mixture;
measuring a first total signal through an optical filter under a first set of conditions, wherein the first total signal includes the first and second detectable signals from the first and second labels, and wherein under the first set of conditions, the first detectable signal is increased as a result of specific interaction of the first probe with the first target, but the second detectable signal is not increased as a result of specific interaction of the second probe with the second target;
measuring a second total signal through the same optical filter under a second set of conditions, wherein the second total signal includes the first and second detectable signals from the first and second labels, and wherein under the second set of conditions, the second detectable signal is increased as a result of specific interaction of the second probe with the second target; and
assessing the presence or amount of the first and/or second target, by estimating the first detectable signal and the second detectable signal based on both the first total signal and the second total signal.

Embodiment 171. The method of embodiment 170, wherein the first and second labels are identical and/or generate substantially identical fluorescence.

Embodiment 172. The method of one of embodiments 170 to 171, wherein the second fluorescence signal differs between the first and second set of conditions to a greater degree than the first fluorescence signal differs between the first and second set of conditions.

Embodiment 173. The method of one of embodiments 170 to 172, wherein the first probe is a cleavable probe.

Embodiment 174. The method of embodiment 173, wherein the first detectable signal increases when the cleavable probe is cleaved.

Embodiment 175. The method of one of embodiments 173 to 174, wherein the first probe comprises a fluorophore and a quencher, and wherein the first probe is configured such that fluorescence from the fluorophore is quenched by the quencher until the probe is cleaved.

Embodiment 176. The method of embodiment 175, wherein the first probe is a TaqMan probe.

Embodiment 177. The method of one of embodiments 170 to 176, wherein the second probe is a non-cleavable probe.

Embodiment 178. The method of embodiment 177, wherein L⁵ forms a stem-loop structure when the second probe is single-stranded.

Embodiment 179. The method of one of embodiments 177 to 178, wherein R⁵⁰ is a fluorophore, and Q and R⁵⁰ are spaced apart from one another such that R⁵⁰ is quenched when the second probe is single stranded and unquenched when the second probe is incorporated into a double stranded amplicon.

Embodiment 180. The method of embodiment 179, wherein both Q and R⁵⁰ are disposed at or near the stem loop portion of the second probe.

Embodiment 181. The method of one of embodiments 170 to 180, wherein a melting temperature (Tₘ) of the first probe and a Tₘ of the second probe are within about 8° C., or about 6° C., or about 4° C., or about 2° C. of each other.

Embodiment 182. The method of one of embodiments 170 to 181, wherein the first set of conditions comprises a first measurement temperature at which the first fluorescence signal is measured, and the second set of conditions comprises a second, different measurement temperature at which the second fluorescence signal is measured.

Embodiment 183. The method of embodiment 182, wherein the first and second measurement temperatures differ by at least about 10° C. or more, about 15° C. or more, about 20° C. or more, about 25° C. or more, or about 30° C. or more.

Embodiment 184. The method of one of embodiments 182 to 183, wherein at least one of the first or second measurement temperatures is a denaturation temperature at which DNA in the reaction mixture is denatured, such as about 90° C. or above.

Embodiment 185. The method of one of embodiments 170 to 184, further comprising thermal cycling of the reaction mixture between two target temperatures for multiple cycles.

Embodiment 186. The method of embodiment 185, wherein the thermal cycling cycles between two target temperatures for at least 5% of, at least 10% of, at least 15% of, at least 20% of, at least 25% of, at least 30% of, at least 35% of, at least 40% of, at least 45% of, at least 50% of, at least 55% of, at least 60% of, at least 65% of, at least 70% of, at least 75% of, at least 80% of, at least 85% of, at least 90% of, or at least 95% of the cycles.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

Current qPCR probe design has the reporter dye on the 5′ end and a quencher molecule at the 3′ end. There are typically 20 to 30 bases between the fluorophore and the quencher. An internal quencher can be placed closer to the fluorophore dye within 6-12 bases between the reporter dye and quencher. This shortened length can provide better quenching and lower background which, in turn, improves accuracy and robustness of the assay. It also can decrease the “waterfall effect” which decreases the assay robustness.

Double quenching places a quencher at an internal site of the probe as well as having a quencher at the 3′ end. Single internal quenching places the internal quencher within the probe sequence. In embodiments, one internal quencher (single quenching) can confer improvements over double quenching, and also over having only one quencher at the 3′ end.

Quencher dye QSY7 has been used for FAM, VIC, ABY, and JUN while QSY21 has been used for AF647, Cy5, AF676, and Cy5.5. The synthesis to make the internal quenchers involves attaching a bifunctional linker “E-Linker” via NHS chemistry to either of the QSY dyes and then coupling to commercially available dU-linker-amine. Once purified, the molecule is then converted to the corresponding phosphoramdite. This dU-QSY phosphoramidite can be incorporated at an internal site replacing T during automated synthesis of probes, such as TaqMan probes.

Internal quenching may be more robust than traditional double quenching, or 3′ end quenching.

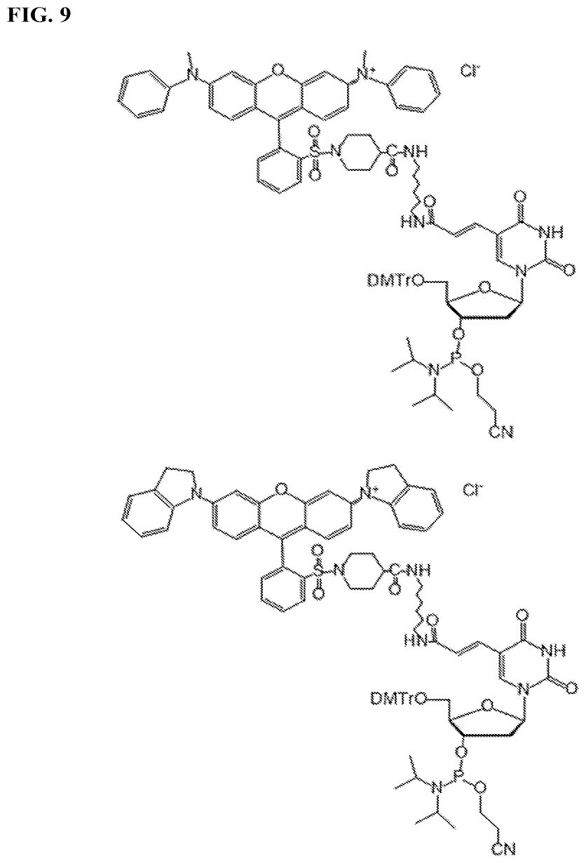

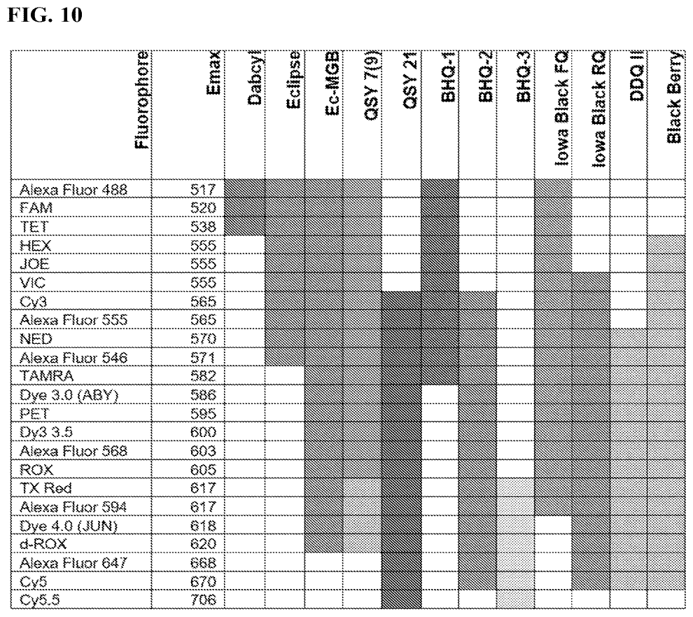

In some embodiments, the structures in FIG. 9 may be used for automation of extendible fluorescent probes.

### Example 2: QSY7 DMT-dU Phosphoramidite Synthesis

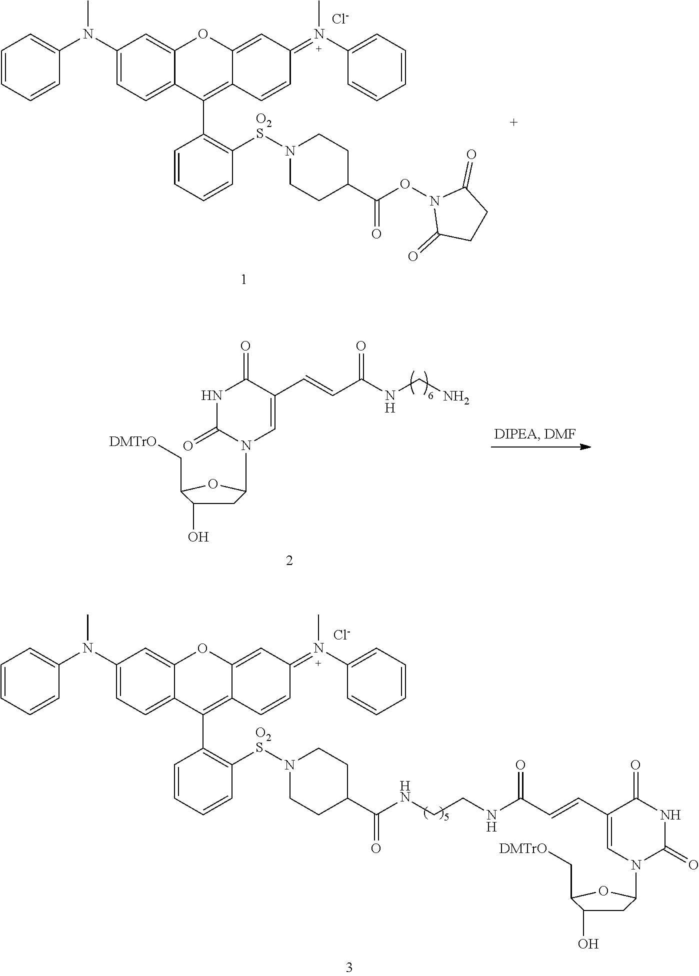

### Step 1: Synthesis of QSY7 DMT-dU 3:

In a flask equipped with a magnet bar, dropping funnel and a septum was added QSY7 NHS ester 1 (7.0 g, 8.84 mmol, 1 equiv.). The set up was purged with Argon for 30 minutes. To the flask was added anhydrous DMF (100 mL) to form a dark purple solution. In a separate flask, a solution of DMT-dU Linker 2 (7.41 g, 10.6 mmol, 1.2 equiv.) was prepared in anhydrous DMF (70 mL) and anhydrous diisopropylethylamine (2.28 g, 3.06 mL, 17.7 mmol, 2 equiv.). The clear solution of DMT-dU Linker 2 was transferred into the dropping funnel and added and mixed to the purple solution of QSY7 NHS ester 1 over 15 minutes at ambient temperature. HPLC analysis of the reaction mixture indicated <5% of QSY-7-NHS ester 1 after 1 hour. The reaction mixture was concentrated to dryness under reduced pressure at 35° C. To the residue was added dichloromethane (containing 1% triethylamine, 300 mL). The dark purple solution was washed with half brine solution (1×200 mL, 2×100 mL). Brine washes were combined and washed with dichloromethane (containing 1% triethylamine, 2×50 mL). All organic phases were combined, dried over sodium sulfate, and concentrated to dryness. To the crude was added acetonitrile (600 ml) and let it mix for 30 minutes at ambient temperature. The resulting thin slurry was added to diethyl ether (3.6 L) while mixing. The mixture was sealed and let it mix at ambient temperature for 30 minutes. The slurry was vacuum filtered over a sintered glass funnel under Argon. The filter cake was aged under argon for 10 minutes. The filter cake was transferred into a flask and dried further under high vacuum for 24 hours to yield QSY7 DMT-dU 3 (10.4 g, 86% isolated yield, 89% purity by HPLC). LCMS calculated 1338.558, observed 1338.559 (M⁺).

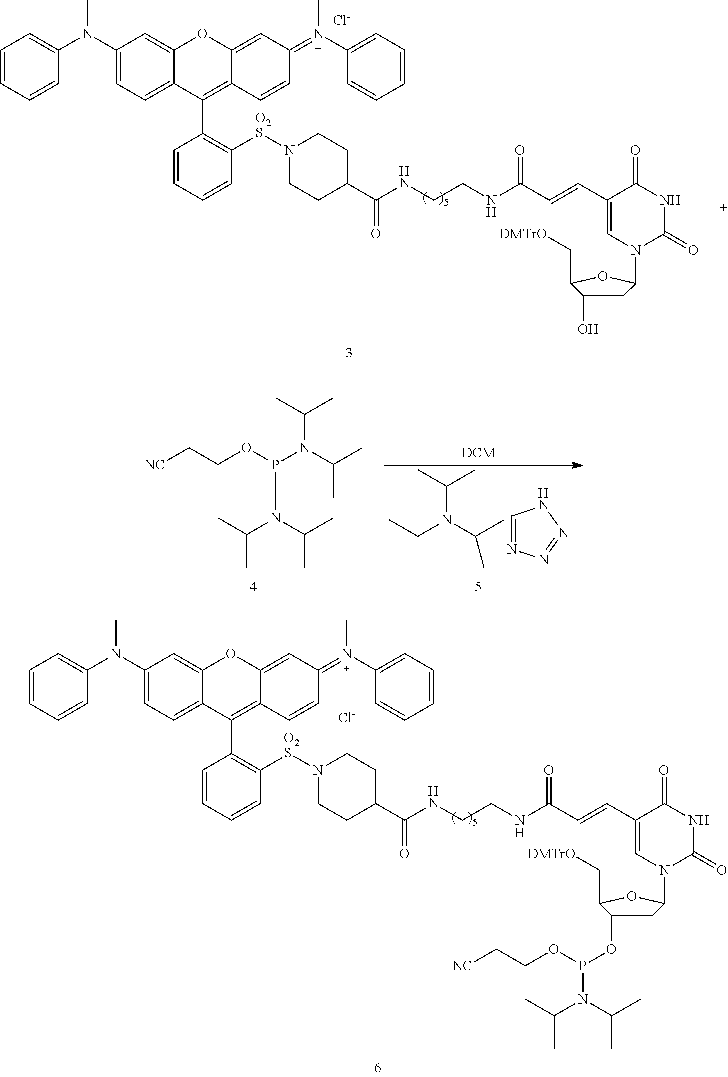

### Step 1: Synthesis of QSY7 DMT-dU Phosphoramidite 6:

In a flask equipped with a magnet bar and septum was charged QSY7 DMT-dU 3 (4.0 g, 2.90 mmol, 1 equiv.). The flask was purged with argon for 15 minutes. To the flask was added anhydrous dichloromethane (140 mL) followed by Tetrazole amine 5 (0.937 g, 7.25 mmol, 2.5 equiv.) and molecular sieves 3 Å (4 g). To the dark purple solution was added 2-cyanoethyl N,N,N′,N′-tetraisopropylphosphorodiamidite 4 (1.31 g, 4.35 mmol, 1.5 equiv.) via syringe over 10 minutes. The reaction mixture was sealed and mixed gently at ambient temperature for 18 hours. HPLC profile of the reaction mixture indicated <5% of QSY7 DMT-dU 3 in the mixture. A normal phase chromatography column (160 g) was pre-treated with dichloromethane/triethylamine (1% v/v) till basic to pH paper. The crude reaction mixture was decanted quantitatively on to the column. The desired product was eluted off the column using dichloromethane/triethylamine (1% v/v) and methanol 2-5%. Fractions with HPLC purity over 85% were combined and concentrated to dryness under reduced pressure at 35° C. Combined fractions were co-evaporated several times from dichloromethane and dried further under high vacuum for 18 hours to yield QSY7 DMT-dU Phosphoramidite 6 (3.43 g, 75% yield, 95% purity by HPLC). ³¹P NMR (400 MHz, DMSO-d6): δ 147.76, 147.32 (d, 1P). LCMS calculated 1539.673, observed 1539.668 (M+1).

### Example 3: QSY21 DMT-dU Amidite Synthesis

### Synthesis of QSY21 DMT-dU Phosphoramidite

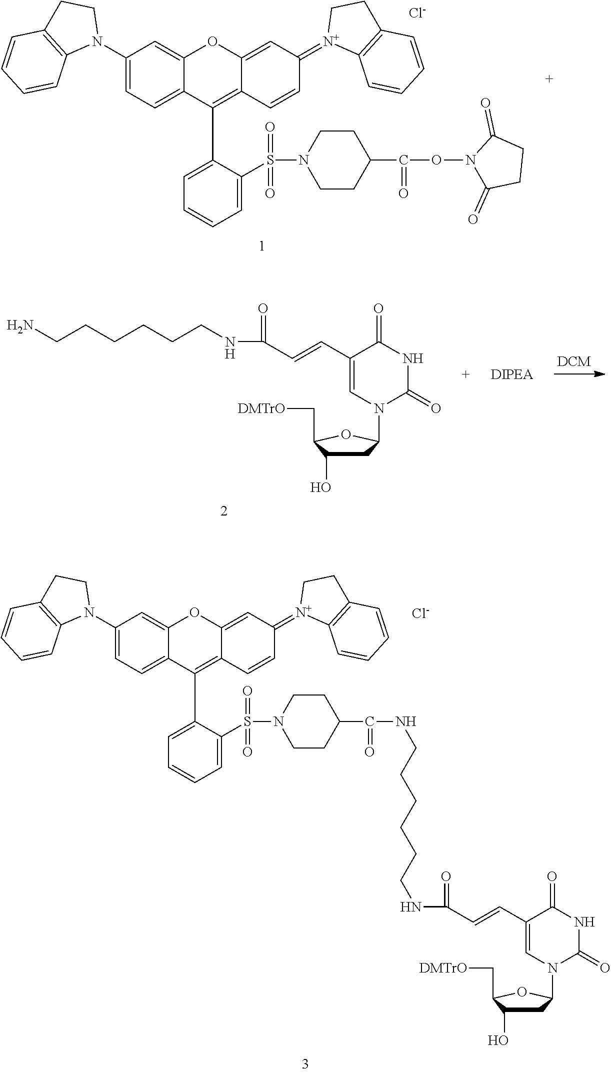

### Step 1: Synthesis of QSY21 DMT-dU (3):

DMT dU Linker (2, 2.83 g, 4.05 mmol, Berry and Associates) was dissolved in 70 mL dry, degassed DMF and then mixed with DIPEA (0.97 g, 7.5 mmol). This was added dropwise over 15 minutes with stirring at room temperature under Ar to QSY21 NHS (1, 2.27 g, 2.78 mmol, Molecular Probes) dissolved in 50 mL of dry, degassed DMF. After stirring for 3 hours, the reaction solution was diluted with 1 L of DCM, washed with 1 L of 1% aq citric acid, followed by 1 L of water, followed by 1 L of brine. The solution was dried over anhydrous sodium sulfate, filtered, and concentrated, co-evaporating with DCM. This was purified by reverse phase column purification eluting stepwise with 80%-90% MeOH/1M triethylammonium bicarbonate to yield 3.12 g (80%) of dark blue solid (3). LCMS calculated 1362.553, observed 1362.556 (M⁺).

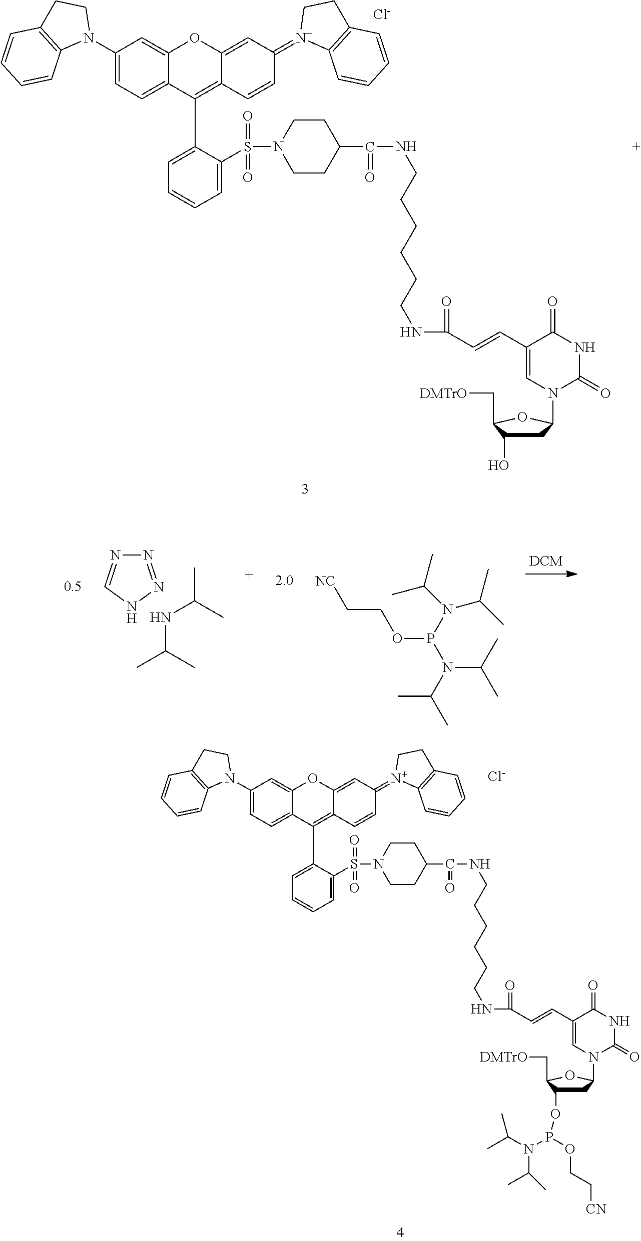

### Step 2: Synthesis of QSY21 DMT-dU Phosphoramidite (4):

QSY21 DMT-dU (3, 3.09 g, 2.21 mmol) was dissolved in 300 mL of anhydrous DCM containing about 20 g of 3 Å sieves. This was placed under nitrogen and gently stirred. Tetrazole amine (0.76 g, 4.42 mmol) was added followed by 2-cyanoethyl N,N,N′,N′-tetraisopropylphosphorodiamidite (2.66 g, 8.84 mmol). The mixture was stirred for 2.5 hours, monitoring for reaction completion by HPLC. The solution was decanted and directly purified by column chromatography on silica gel that was pre-equilibrated with 1% triethylamine eluting stepwise with 0/5/10/15% MeOH/DCM containing 1% triethylamine. The pooled fractions were concentrated, co-evaporated several times in DCM, and dried under vacuum to yield 3.59 g (102%) of dark blue foamed solid (4). ³¹P NMR (400 MHz, CD₂Cl₂): δ 148.87, 148.78 (d, 1P). Quantitative ³¹P NMR (400 MHz, CD₂Cl₂, PPh₃ as quantitative reference): 95% purity. LCMS calculated 1562.666, observed 1562.663 (M⁺).

Oligo Synthesis with Internal Quencher

### Generic Scheme of Internal Quenching Using QSY21:

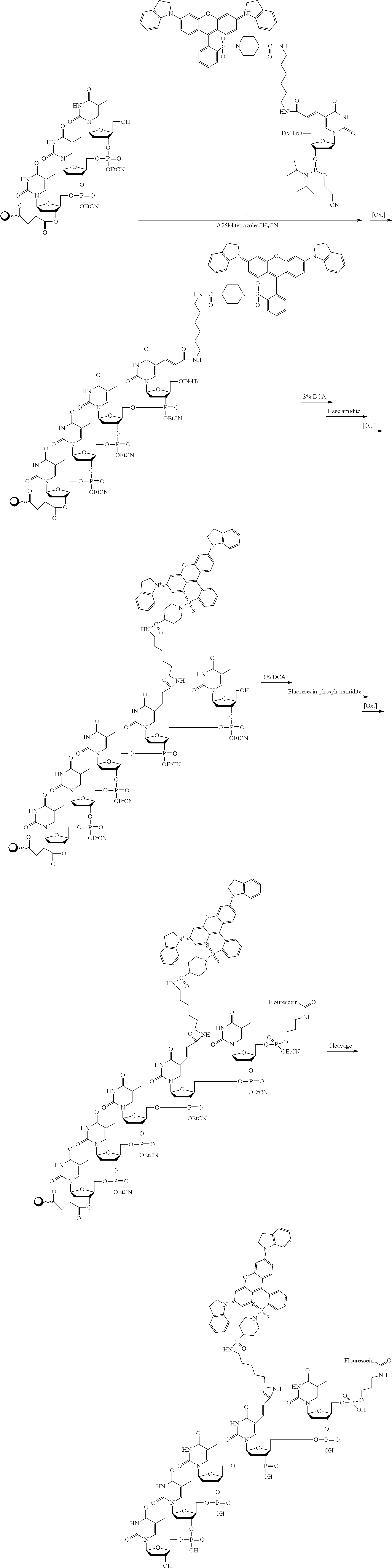

Oligonucleotides internally labeled with the QSY7 (or 21) DMT-dU phosphoramidite reagent were synthesized on polystyrene solid supports using the standard operating conditions on a Biolytic 3900 automated DNA synthesizer. The QSY7 (or QSY21) DMT-dU phosphoramidite was dissolved in acetonitrile or DCM-acetonitrile (1:1) solvent for the coupling reactions and was labeled at the designated oligo location growing from the 3′ end. The fluorescent reporter dye was then labeled at the 5′ end. The resultant labeled oligo probe was cleaved and deprotected from the support to yield reporter dye labeled oligonucleotide with the quencher labeled internally.

### Example 4: Detecting Nucleic Acids Using Intra-Channel Multiplexing

Nucleic acid detection assays are often carried out by adding a sample that is suspected of including one or more target nucleic acids to a reaction mixture. The reaction mixture also includes one or more detectable labels each designed to associate with a different target nucleic acid and generate a signal that corresponds to the amount of target nucleic acid in the reaction mixture. In a “singleplex” assay, the reaction mixture includes a single detectable label designed to associate with a single target. Conversely, in a “multiplex” assay, the reaction mixture includes multiple, different detectable labels each typically designed to be specific to a different target. Multiplex assays are therefore capable of detecting multiple different targets in a single reaction mixture. Often, the detectable labels are fluorescent dyes integrated with a nucleic acid probe, a primer, or some other nucleic acid molecule designed to specifically hybridize with the corresponding target nucleic acid.

In conventional multiplex assays, each dye is assigned to a different target. The presence and/or amount of each target can then be determined by measuring the fluorescence signal in separate “detection channels” each corresponding to the emission wavelength of the corresponding dye. However, as shown, there is typically a substantial amount of overlap in the emission spectra of the dyes. Increased overlap in emission spectra increases the difficulty in resolving the separate fluorescence signals and thus increases the difficulty in detecting and/or quantifying the respective targets. Excessive overlap can require, for example, complex deconvolution algorithms to sufficiently resolve the separate fluorescence signals.

While multiplexed dyes are typically selected with the intent to minimize spectral overlap, the finite nature of the emission spectrum places practical limits on the number of separate dyes that can be combined in the same multiplex assay, at least without resorting to increasingly complex reaction protocols and backend deconvolution requirements. As a result, at present, there are significant limitations to the number of different targets that can be detected and/or measured in a multiplex assay. Accordingly, there is an ongoing need for compositions and methods capable of increasing the “plexy” of detection assays.

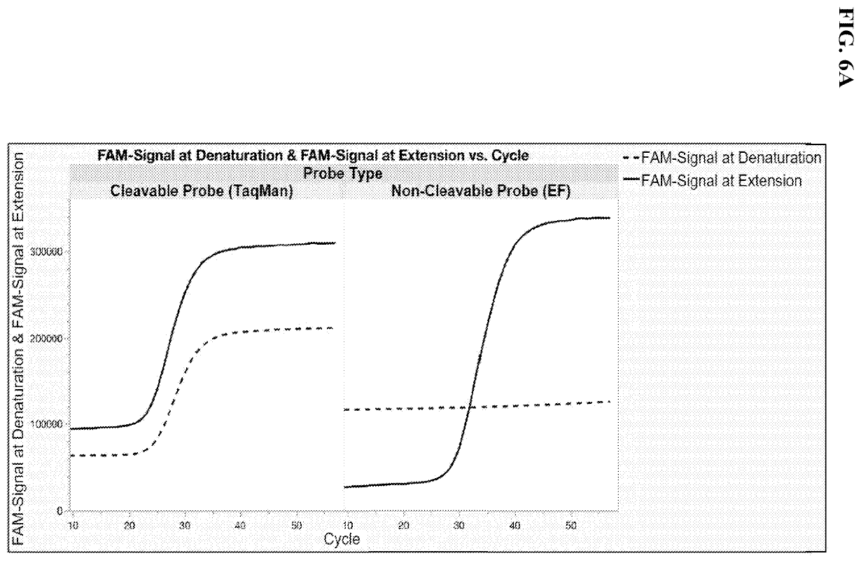

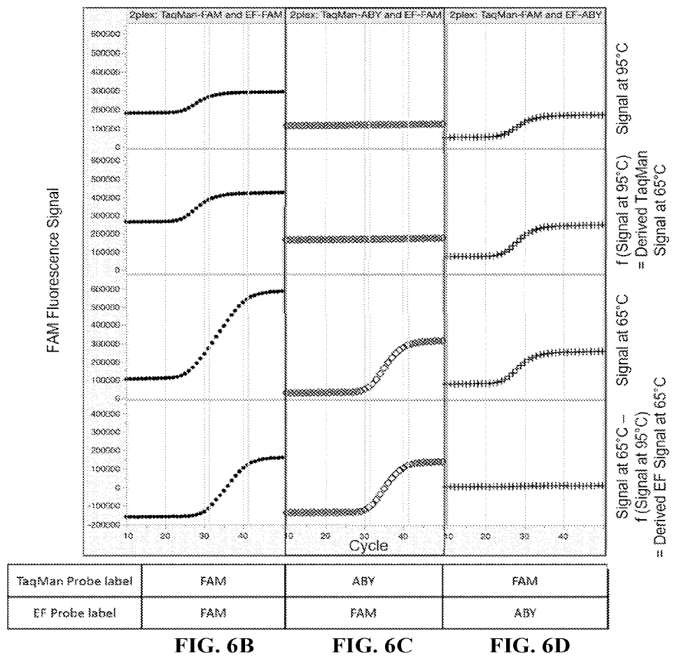

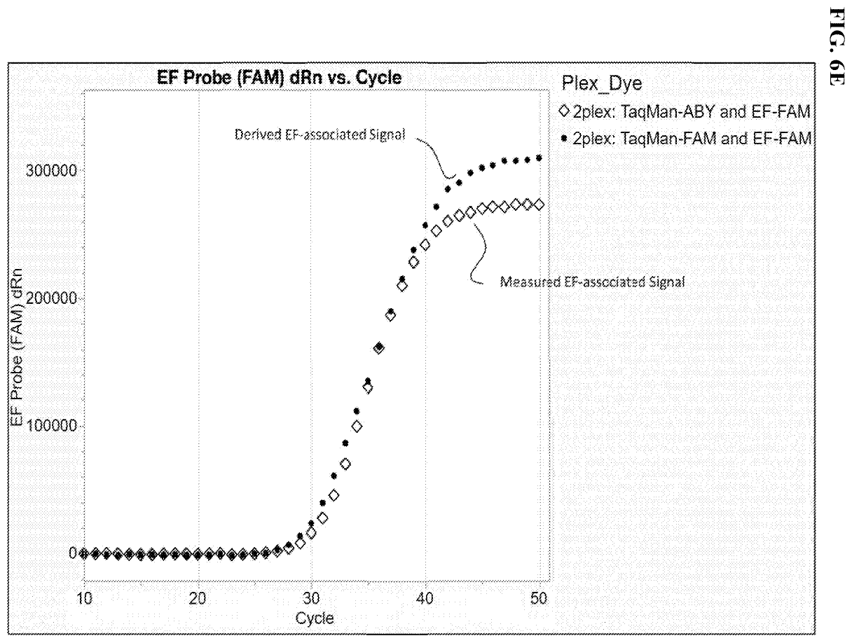

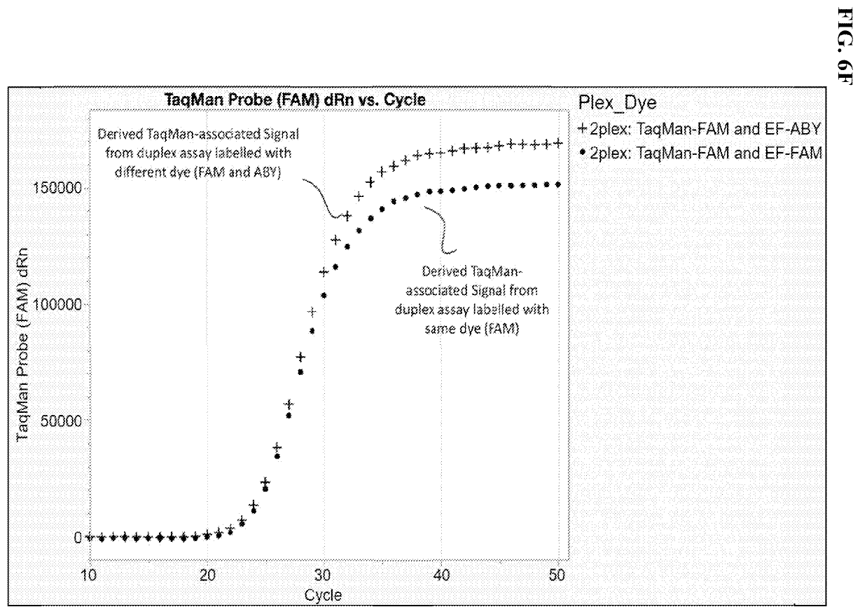

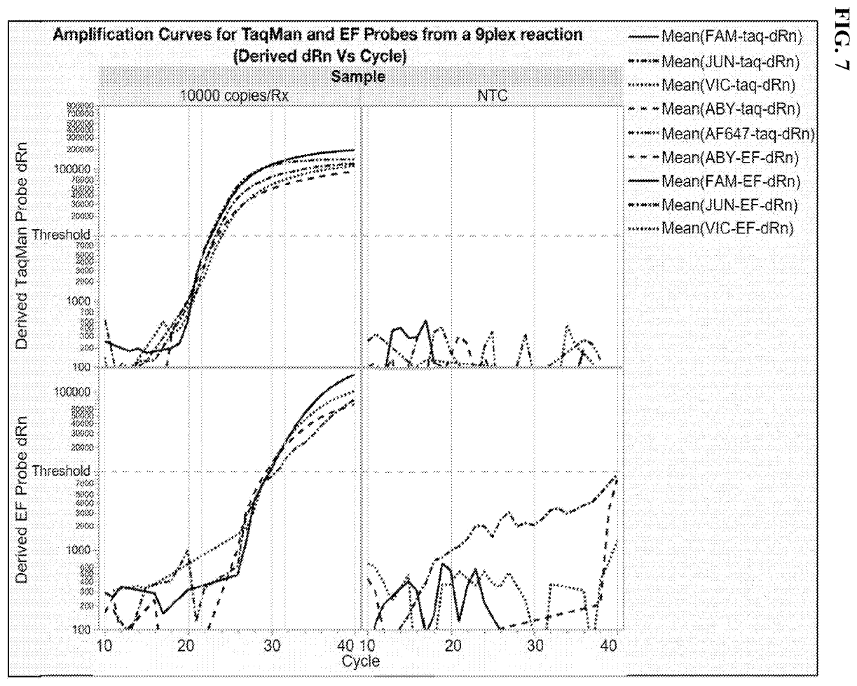

FIG. 6A illustrates the fluorescence signal over cycle number measured at the annealing/extension temperature (65° C. in this example) and at the denaturation temperature (95° C. in this example) with TaqMan probe and EF probe compositions.

FIGS. 6B and 6C illustrate results of qPCR duplex assay tests, measuring fluorescence signal in the FAM channel, in in which TaqMan and EF probes were designed to generate fluorescence signals in the same dye channel (FIG. 6B) or in different dye channels (FIGS. 6C-6D). In the assay shown in FIG. 6B, both the TaqMan probes and the EF probes were labelled with FAM. In the assay shown in FIG. 6C, the TaqMan probes were labelled with ABY and the EF probes were labelled with FAM. In the assay shown in FIG. 6D, the TaqMan probes were labelled with FAM and the EF probes were labelled with ABY. The reaction mixture composition, template DNA concentrations, and amplification conditions were otherwise held the same across each assay.

In FIG. 6B, the top row shows the FAM channel fluorescence signal over cycle number measured at the denaturation temperature (95° C. in this example). This signal is expected to include fluorescence generated mostly by TaqMan probe labels (those that have been cleaved from the probes). The second row shows the fluorescence signal over cycle number measured at the denaturation temperature (95° C. in this example) and modified by a linear function that correlates the 95° C. measurement to a 65° C. measurement for the TaqMan probes. This signal is expected to include fluorescence generated by the TaqMan probe labels but not to include significant fluorescence from the EF probe labels. The third row shows the fluorescence signal over cycle number measured at the annealing/extension temperature (65° C. in this example). This signal is expected to include fluorescence generated by both the TaqMan probe labels (those that have been cleaved from the probes) and the EF probe labels (those that have been incorporated into double stranded amplicons). The bottom row shows the resolved fluorescence signal determined by subtracting the second row signal from the third row signal. This signal is expected to estimate the fluorescence generated by the EF probe labels, separate from fluorescence attributable to the TaqMan probe labels.

In FIG. 6C, the top, second, third, and bottom rows represent the same signal measurement types as in FIG. 6B. As shown on the top and second rows, the EF probe labels (FAM) generated insignificant (essentially zero) fluorescence at the denaturation temperature. Because the TaqMan and EF probes were differentially labelled in this assay, the bottom row shows a resolved signal for the EF probe label that essentially matches the EF probe signal at the annealing/extension temperature (third row).

In FIG. 6D, the top, second, third, and bottom rows represent the same signal measurement types as in FIG. 6B but with TaqMan probe labelled with FAM and EF probe labelled with ABY. The first-row fluorescence signal is mostly generated by the TaqMan probe label (those that have been cleaved from the probes). The second row shows the fluorescence signal over cycle number measured at the denaturation temperature (95° C. in this example) and modified by a linear function that correlates the 95° C. measurement to a 65° C. measurement for the TaqMan probes. Because the TaqMan and EF probes were differentially labelled in this assay, the derived TaqMan signal (second row) essentially matches the measured signal at the annealing/extension temperature (third row), and the resolved signal for EF probe label (bottom row) is essentially zero.

FIG. 6E compares the resolved EF-associated fluorescence signal after baseline adjustment (ΔRn) (bottom row of FIG. 6B) with the EF-associated fluorescence signal after baseline adjustment (ΔRn) (bottom row of FIG. 6C), which represents a direct measurement of EF probe label fluorescence. The results showed close correlation between the resolved and measured signals. The results therefore showed that fluorescent signals attributable to different probe types within the same detection channel can be separately resolved.

FIG. 6F compares the derived TaqMan-associated fluorescence signal after baseline adjustment (ΔRn) (second row of FIG. 6B) with the derived TaqMan-associated fluorescence signal after baseline adjustment (ΔRn) (second row of FIG. 6D). The results showed close correlation between the derived TaqMan signals from separate assays where the EF probes are similarly labelled (FIG. 6B) or labelled with a different dye (FIG. 6D).

FIG. 7 illustrates the results of another assay test that included 5 different detection channels/dyes, each with a corresponding TaqMan probe and an EF probe, the results showing that the fluorescent signals of the different probe types can be independently determined, and showing that a 9-plex reaction can be effectively carried out.

FIG. 8 is a plot comparing the endpoint signal of partitions at 65° C. and at 95° C. following a dPCR process. As shown, the partition signals fall into identifiable clusters. The clusters may be estimated using cluster analysis algorithms known in the art. In the Figure, the “EF” cluster represents those partitions that provide a signal at the annealing/extension temperature but have limited signal at the denaturation temperature, the “T” cluster represents those partitions that provide a signal at both the annealing/extension temperature and the denaturation temperature, and the “T+EF” cluster represents those partitions that provide a signal at the denaturation temperature and a heightened signal at the annealing/extension temperature. The total count of partitions in which the TaqMan probes generated a signal equals the count of cluster T added to the count of cluster T+EF, and the total count of partitions in which the EF probes generated a signal equals the count of cluster EF added to the count of cluster T+EF. Concentrations of the first and second target in the sample may then be estimated using standard dPCR techniques.

1. An oligonucleotide, or a salt thereof, having the formula: 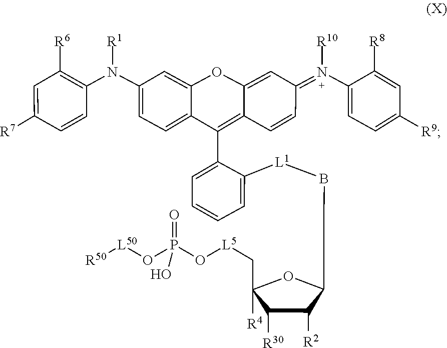
B is a divalent nucleobase;
L¹ is a divalent linker L¹⁰¹-L¹⁰²-L¹⁰³-L¹⁰⁴-L¹⁰⁵; and
L¹⁰¹, L¹⁰², L¹⁰³, L¹⁰⁴, and L¹⁰⁵ are independently a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L⁵ is a divalent oligonucleotide linker comprising from 4 to 40 nucleotides;
L⁵⁰ is a bond, —NH—, —O—, —S—, —S(O)—, —S(O)₂—, —C(O)—, —C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵⁰ is a detectable moiety selected from the group consisting of a monovalent form of FAM, a monovalent form of VIC, a monovalent form of ABY, a monovalent form of JUN, a monovalent form of AF647, a monovalent form of Cy5, a monovalent form of AF676, or a monovalent form of Cy5.5;
R³⁰ is —OR^{30A};
R^{30A} is hydrogen or a monovalent oligonucleotide moiety;
R² is hydrogen or —OR^{2A};
R⁴ is hydrogen or unsubstituted methyl, or R² and R⁴ substituents are joined to form a substituted or unsubstituted heterocycloalkyl;
R¹ and R¹⁰ are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R⁶, R⁷, R⁸, and R⁹ are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃R^{A}, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹ and R⁶ may be joined to form a substituted or unsubstituted heterocycloalkyl or substituted or unsubstituted heteroaryl;
R⁸ and R¹⁰ may be joined to form a substituted or unsubstituted heterocycloalkyl or substituted or unsubstituted heteroaryl; and
R^{2A}, R^{3A}, R^{3B}, R^{3C}, and R^{A} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

2. The oligonucleotide of claim 1, having the formula:

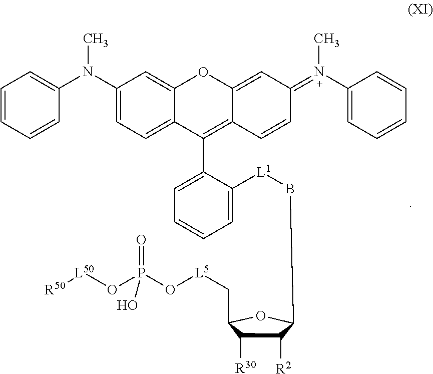

3. The oligonucleotide of claim 1, having the formula:

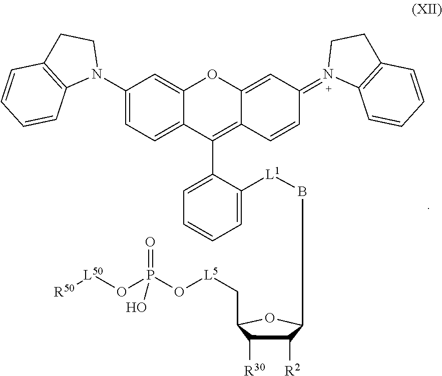

4. The oligonucleotide of claim 1, having the formula:

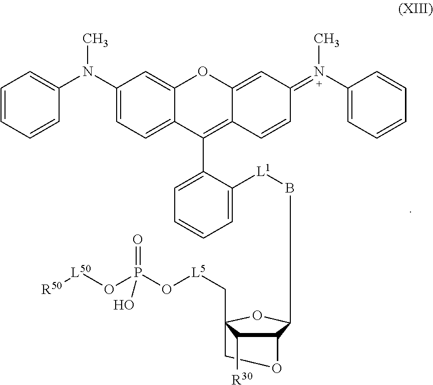

5. The oligonucleotide of claim 1, having the formula:

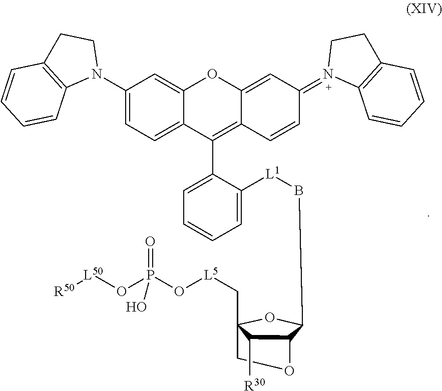

6. The oligonucleotide of claim 1, wherein B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof.

7. The oligonucleotide of claim 1, wherein B is a divalent cytosine or a derivative thereof, divalent guanine or a derivative thereof, divalent adenine or a derivative thereof, divalent thymine or a derivative thereof, or divalent uracil or a derivative thereof.

8. The oligonucleotide of claim 1, having the formula:

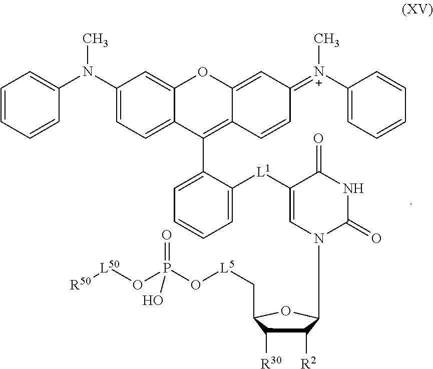

9. The oligonucleotide of claim 1, having the formula:

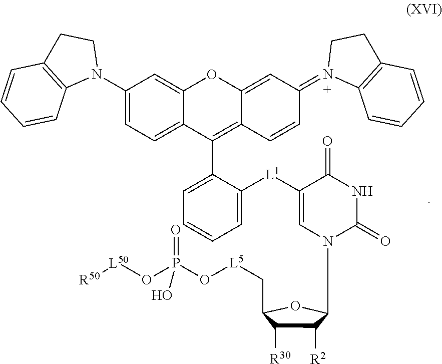

10. The oligonucleotide of claim 1, having the formula:

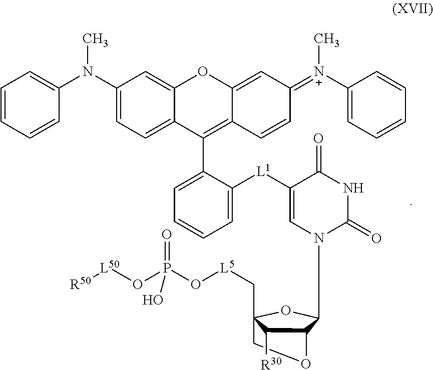

11. The oligonucleotide of claim 1, having the formula:

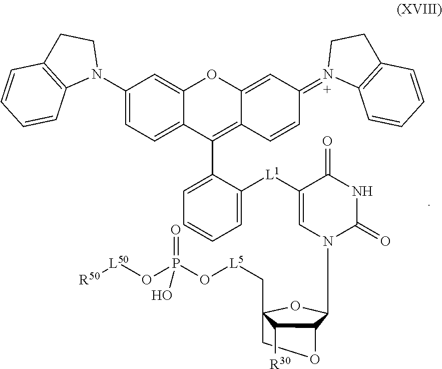

12. (canceled)

13. (canceled)

14. The oligonucleotide of claim 1, wherein L⁵ comprises from 4 to 14 nucleotides.

15. The oligonucleotide of claim 1, wherein L⁵ comprises from 6 to 12 nucleotides.

16. The oligonucleotide of claim 1, wherein the nucleotides are DNA nucleotides.

17. The oligonucleotide of claim 1, wherein the nucleotides are RNA nucleotides.

18. (canceled)

19. (canceled)

20. The oligonucleotide of claim 1, wherein L¹⁰² is an unsubstituted 3 to 8 membered heterocycloalkyl.

21. (canceled)

22. (canceled)

23. (canceled)

24. The oligonucleotide of claim 1, wherein L¹⁰⁴ is an unsubstituted C₁-C₁₀ alkylene, unsubstituted 2 to 6 membered heteroalkylene, or unsubstituted phenylene.

25. (canceled)

26. The oligonucleotide of claim 1, wherein L¹⁰⁵ is an unsubstituted C₁-C₁₀ alkylene, substituted or unsubstituted 2 to 8 membered heteroalkylene, or unsubstituted 5 to 10 membered heteroarylene.

27. The oligonucleotide of claim 1, wherein L¹⁰⁵ is

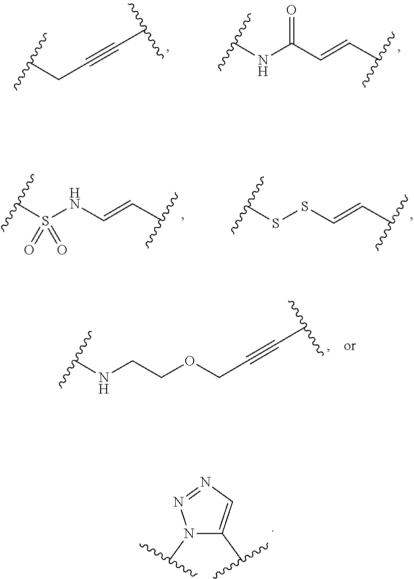 [SMILES: *C#CC* ; */C=C/C(=O)N* ; */C=C/NS(*)(=O)=O ; */C=C/SS* ; *C#CCOCCN* ; *c1cnnn1*]

28. The oligonucleotide of claim 1, wherein L¹ is

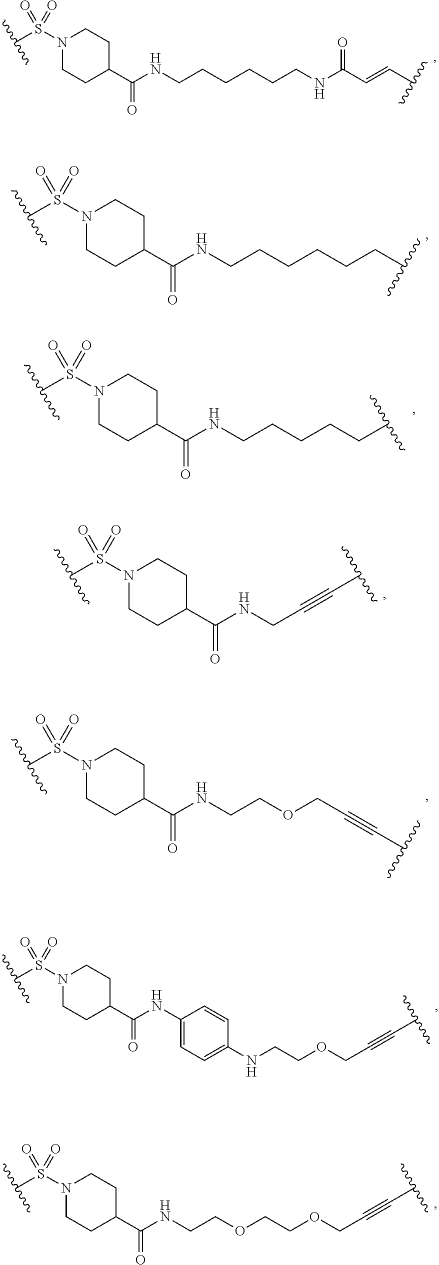

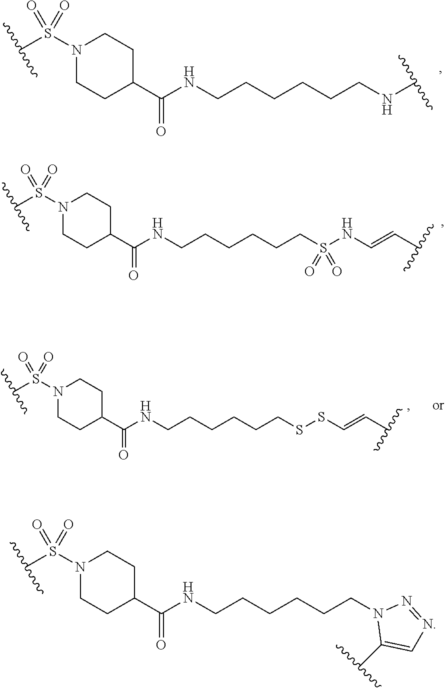 [SMILES: *NCCCCCCNC(=O)C1CCN(S(*)(=O)=O)CC1 ; */C=C/NS(=O)(=O)CCCCCCNC(=O)C1CCN(S(*)(=O)=O)CC1 ; */C=C/SSCCCCCCNC(=O)C1CCN(S(*)(=O)=O)CC1 ; *c1cnnn1CCCCCCNC(=O)C1CCN(S(*)(=O)=O)CC1]

29-186. (canceled)
